(19) United States
(12) Patent Application Publication
(10) Pub. No.: US 2025/0237650 A1
(43) Pub. Date: Jul. 24, 2025

(54) GLYCOSAMINOGLYCAN ARTICLES AND METHODS RELATING THERETO

(71) Applicants: The University of North Carolina at Chapel Hill, Chapel Hill, NC (US); THE REGENTS OF THE UNIVERSITY OF CALIFORNIA, Oakland, CA (US)

(72) Inventors: Ronit Fraiman, Chapel Hill, NC (US); Sanghoon Kim, Raleigh, NC (US); Rommie Elizabeth Amaro, San Diego, CA (US); Fiona Lynn Kearns, Tampa, FL (US)

(21) Appl. No.: 18/702,066

(22) PCT Filed: Nov. 10, 2022
(86) PCT No.: PCT/US2022/079641

§ 371 Date: Apr. 17, 2024

Related U.S. Application Data
(60) Provisional application No. 63/277,630, filed on Nov. 10, 2021.
(60) Provisional application No. 63/379,952, filed on Oct. 18, 2022.

(51) Int. Cl. G01N 33/569; G01N 33/543

(52) U.S. Cl. CPC G01N 33/56983; G01N 33/54387; G01N 2333/165; G01N 2400/40; G01N 2469/10

(57) ABSTRACT

Described herein are glycosaminoglycan articles (e.g., test strips, detection systems, and binding pairs) along with methods of making and using the same.

### STATEMENT OF FEDERAL SUPPORT

This invention was made with government support under Grant Number GM132826 awarded by the National Institutes of Health and Grant Numbers MCB-2032054 and DMS-2028758 awarded by the National Science Foundation. The government has certain rights in the invention.

### FIELD

The present invention relates to glycosaminoglycan articles (e.g., test strips, detection systems, and binding pairs) along with methods of making and using the same.

### BACKGROUND

New tests and methods are needed to detect pathogens. In particular, new tests and methods are needed to rapidly detect pathogens in a non-invasive fashion without the need for specialized equipment or personnel.

### SUMMARY

A first aspect of the present invention is directed to a test strip comprising: a substrate including a test region, wherein the test region comprises a glycosaminoglycan (GAG) (e.g., a heparan sulfate proteoglycan (HSPG)) attached to the substrate. In some embodiments, the substrate is configured to move a fluid sample by capillary action through the substrate to the test region.

Another aspect of the present invention is directed to a kit comprising: a test strip that comprises a substrate including a test region, wherein the test region comprises a glycosaminoglycan (GAG) (e.g., a heparan sulfate proteoglycan (HSPG)) attached to the substrate; and a detection agent configured to bind a pathogen. In some embodiments, the substrate is configured to move a fluid sample by capillary action through the substrate to the test region.

A further aspect of the present invention is directed to a method of detecting a pathogen in a fluid sample, the method comprising: contacting a test strip and a fluid sample, wherein the test strip comprises a substrate including a test region, wherein the test region comprises a glycosaminoglycan (GAG) (e.g., a heparan sulfate proteoglycan (HSPG)) attached to the substrate; and detecting a first signal at the test region, wherein the first signal indicates the presence of the pathogen in the fluid sample, thereby detecting the pathogen in the fluid sample. In some embodiments, the substrate is configured to move a fluid sample by capillary action through the substrate to the test region.

An additional aspect of the present invention is directed to a glycosaminoglycan (GAG) that is optionally attached to a substrate and/or to a signaling agent. In some embodiments, the GAG is attached to a substrate to which a composition optionally comprising a target (e.g., a pathogen) is contacted and the substrate is immobile in the composition. In some embodiments, the GAG is mobile in a composition that optionally comprises a target (e.g., a pathogen) and the GAG may be attached to a signaling agent.

Another aspect of the present invention is directed to a binding pair. In some embodiments, the binding pair comprises: a first member of the binding pair, wherein the first member comprises a glycosaminoglycan (GAG); and a second member of the binding pair, wherein the second member comprises a detection agent that includes a signaling agent, wherein the first member and the second member are each configured to bind to the same target. In some embodiments, the binding pair comprises: a first member of the binding pair, wherein the first member comprises a glycosaminoglycan (GAG) attached to a signaling agent; and a second member of the binding pair, wherein the second member comprises a compound (e.g., a protein (e.g., an antibody), a peptide, a polysaccharide (e.g., a GAG), a nucleic acid, and/or any combination and/or fragment thereof) that optionally includes a signaling agent, wherein the first member and the second member are each configured to bind to the same target. In some embodiments, the second member is devoid of a signaling agent.

Further aspects of the present invention are directed to compositions, kits, test strips, and systems (e.g., detection systems) comprising a binding pair of the present invention.

An additional aspect of the present invention is directed to a method of detecting a target in a fluid sample, the method comprising: combining a fluid sample and a binding pair of the present invention, optionally wherein the fluid sample comprises the target; and detecting formation of a complex comprising the first member, the second member and the target, thereby detecting the target in the fluid sample.

Another aspect of the present invention is directed to a solution comprising a glycosaminoglycan (GAG) (e.g., a heparin and/or heparan sulfate proteoglycan (HSPG)) attached to a first material (e.g., a metal particle such as a gold nanoparticle), and a detection agent (e.g., a protein such as an antibody, enzyme, etc.) including a second material. The first material may be a first signaling agent and/or the second material may be a second signaling agent, and the first and second signaling agents may be the same or different. In some embodiments, the solution may be used to detect a pathogen present in a fluid sample, the method comprising combining the solution and fluid sample and detecting the pathogen in the fluid sample.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is an illustration of a test strip a test strip 10 according to some embodiments of the present invention.

FIG. 2 is an illustration of a test strip a test strip 20 according to some embodiments of the present invention.

FIG. 3 is an illustration of virus interaction with GAGs on a cell surface.

FIG. 4 is an illustration of a GlycoGrip lateral flow (LF) biosensor for detecting SARS-CoV-2 according to some embodiments of the present invention. The sample is deposited on the sample pad and migrates towards the conjugation pad that includes anti-spike antibodies attached to gold nanoparticles. The antibodies bind the virus (e.g., target analyte) and migrate to the test line, where the bound virus is captured by the glycopolymers attached at the test line. The antibodies can also be captured by anti-IgG antibodies attached at the control line.

FIG. 5 is a molecular representation of GAGs: HEP, HS (sulfated at the C6 position), CS, and DEX. All four GAGs were modeled in both dimeric (n=1) and tetrameric (n=2) forms.

FIG. 6 shows experimentally calculated binding affinities between spike and tested GAGs (steady-state analysis of BLI data to determine K_{D} values).

FIG. 7 is a schematic illustration of screening of various signaling probe candidates on HEP-based LF strip.

FIG. 8 is a graph showing the screening results with 30 min and 5 min incubation.

FIG. 9 is the LF strip at 30 minutes showing the test line (TL) and control line (CL).

FIG. 10 is the LF strip at 5 minutes showing the test line (TL) and control line (CL).

FIG. 11 is an image and graphs of accessible surface area calculated from the RBD epitope (REGN10933), ACE2 binding motif, and NTD epitope (4A8). Dark blue bars indicate the size of the interface area as seen in Cryo-EM structures for the RBD, ACE2, and NTD binding footprints (6XDG, 6M17, and 7C2L, respectively).

FIG. 12 is a graph of a computational calculation of binding energy of hep8mer to spike RBD over a range of implicit solvent ionic strengths.

FIG. 13 is a graph of BLI results of HEP to spike in three different concentrations of NaCl (10 mM, 75 mM, 150 mM).

FIG. 14 is a graph showing of the response of the lateral flow test in different ionic concentrations (10 mM, 75 mM, 150 mM).

FIG. 15 is a graph showing the screening results of HS15, HEP15, and HEP27 using LFSA. p values<0.05 (*), 0.01 (**) and 0.001 (***) determined using a one-way ANOVA with Tukey's post hoc test.

FIG. 16 shows analytical performance of HEP15 based GlycoGrip LF biosensor in buffer conditions.

FIG. 17 shows selectivity of HEP15 based GlycoGrip LF biosensor was tested with different counter targets: SARS-CoV, MERS-CoV, ACE2, human serum albumin (HSA), and bovine serum albumin (BSA).

FIG. 18 is a schematic illustration of the signal enhancement using HRP and AEC.

FIG. 19 shows the analytical performance of the signal enhanced GlycoGrip LF biosensor in human saliva condition.

FIG. 20 shows BLI results of the HEP to spike proteins (Wild type, Alpha (B.1.1.7), Beta (B.1.351), and Delta (B.1.617.2).

FIG. 21 shows the response of the Alpha, Beta, and Delta variants in GlycoGrip LF biosensor. Statistical analysis was performed using a one-way ANOVA with Tukey's post hoc test.

FIG. 22 shows an image of tests strips according to some embodiments of the present invention and a graph providing the normalized signal intensity for the tested concentrations on the respective test strips.

FIG. 23 Panel A is a schematic depicting initiation of host-cell invasion process through HS/ACE2/spike ternary complex formation via (1) SARS-CoV-2 approach to the human host-cell, (2) SARS-CoV-2 viral binding to Heparan Sulfate Proteoglycans (HSPGs) on the host-cell membrane, (3) conformational change of SARS-CoV-2 spike proteins from a closed to open state, open spike demonstrating 1 Receptor Binding Domain (RBD) in the “up” state with an exposed RBM, i.e. a “1 up” spike, and finally (4) spike/ACE2 binding mediated by the Receptor Binding Motif (RBM). Panel B shows molecular models depicting steps 3 and 4 from Panel A.³³ Inset (i) in Panel B illustrates an apical view of the spike head highlighting the RBM's relative exposure in closed and open spike conformations.

FIG. 24 Panel A is a graph showing the binding affinities of VOC spikes to dimeric ACE2 measured by BLI, K_{D} values were calculated with steady-state analysis. Panel B is a graph showing the degree of bound complex formation for VOC spikes to dimeric ACE2 as measured by ELISA. TMB (3,3′,5,5′-tetramethylbenzidine) was used as a chromogen for ELISA and an illustration showing the process of binding of VOC spikes to dimeric ACE2. Panel C is a graph showing the binding affinities of VOC spikes to HS measured by BLI, K_{D} values calculated with steady-state analysis. Panel D is a graph showing the degree of bound complex formation for VOC spikes to HS as measured by ELISA and an illustration showing the process of binding of VOC spikes to HS. Three independent tests were performed (n≥3), and p values<0.05 (*), 0.01 (**) and 0.001 (***) were determined using a one-way ANOVA with Tukey's post hoc test.

FIG. 25 is a graph showing the rate constant (kₒₙ) to b-surface calculated between heparin and heparin sulfate tetramer to WT, Delta, and Omicron spike proteins (each titrated to pH 7.4). When calculating rate constants (kₒₙ) to the b-surface, receptor molecules are modeled as spheres defined by a b-radius, and total charge. The kₒₙ is calculated analytically according to the Smoluchowski equation. Bars are colored according to a red to blue color scale normalized to VOC spike total charge with 14 sialilated glycans: WT (−11) in red, Delta (+4) in light blue, and Omicron (+10) in blue.^{56,82}

FIG. 26 is a graph showing the rate constant (kₒₙ) to b-surface calculated between and the ACE2 exodomain (residues 18 to 734, pH 7.4). When calculating rate constants (kₒₙ) to the b-surface, receptor molecules are modeled as spheres defined by a b-radius, and total charge. The kₒₙ is calculated analytically according to the Smoluchowski equation. Bars are colored according to a red to blue color scale normalized to VOC spike total charge with 14 sialilated glycans: WT (−11) in red, Delta (+4) in light blue, and Omicron (+10) in blue.^{56,82}

FIG. 27 is a graph showing the second-order rate constants (kₒₙ) calculated between the receptor binding domain (RBD) cleft, RBD patch, furin cleavage site (FCS), and the receptor binding motif (RBM) of the spike structure and the heparin sulfate tetramer. The kₒₙ is calculated analytically according to the Smoluchowski equation. Bars are colored according to a red to blue color scale normalized to VOC spike total charge with 14 sialilated glycans: WT (−11) in red, Delta (+4) in light blue, and Omicron (+10) in blue.^{56,82}

FIG. 28 is a graph showing the degree of bound ternary complex formation for VOC spikes to HS and ACE2 as measured by ELISA and an illustration showing the process of binding of VOC spikes to HS. Three independent tests were performed (n≥3), and p values<0.05 (*), 0.01 (**) and 0.001 (***) were determined using a one-way ANOVA with Tukey's post hoc test.

FIG. 29 Panel A is a graph showing mass photometer results comparing the WT, Delta, Omicron binding to ACE2 and long-chain HS. (i) Mass distribution of WT, Delta, and Omicron Spike (mass range highlighted in grey) (ii) Mass distribution of the dACE2 (mass range highlighted in red), (iii) Mass distribution of Spike protein+ACE2, and (iv) Mass distribution of Spike+HS+ACE2. To mimic the viral entry mechanism, HS was incubated with spike proteins first then followed by addition of ACE2. Possible ternary complexes are shown as illustrations grouped in A, B, and C based on their expected mass ranges. The molar ratio used in this study was Spike: HS:ACE2=1:1:0.5. Panel B is a graph that shows the fraction of ternary complex with or without HS for WT, Delta, and Omicron obtained by mass photometer. To calculate the fraction of these complexes, count numbers from each group (A, B, C) in Panel A were obtained. At least three independent experiments were performed, and error bars were calculated by the standard deviation of all experiments. Significance was calculated via multiple t test (unpaired) with Holm-Sidak method (a: 0.05) was performed.

FIG. 30 Panel A inset (i) shows an image of a GlycoGrip prototype with callout image depicting a spike protein bound to both HEP (the GlycoGrip capture agent) and ACE2 (the signaling probe). Panel A inset (ii) is a schematic illustrating GlycoGrip's capture and signaling of SARS-CoV-2 virions. Panel B shows both test strips and a corresponding graph of test strip signal intensities showing the comparison of the GlycoGrip1.0 and GlycoGrip2.0 to VOCs with two different signaling probes (NTD Ab and ACE2; NTD Ab was previously used in GlycoGrip1.0). Panel C is a graph showing the correlation between relative GlycoGrip1.0 signal intensity versus change in spike charge; charge change calculated as VOC spike charge WT spike charge, relative signal intensities plotted as ratio with respect to WT (same data as shown in Panel B (NTD)). Panel D is a graph showing the correlation between relative GlycoGrip2.0 signal intensity versus change in spike charge; charge change calculated as VOC spike charge-WT spike charge, relative signal intensities plotted as ratio with respect to WT (same data as shown in Panel B (ACE2)). Panel E shows both test strips and a corresponding graph of test strip signal intensities showing the selectivity of the GlycoGrip2.0 to relevant proteins including MERS, CoV1 spike, HIV envelope protein (gp140), Humans serum albumin (HSA), and human saliva. Panel F shows both test strips and a corresponding graph of test strip signal intensities showing the dose-dependency results of Omicron detection using GlycoGrip2.0 with signal enhancement. The limit of the detection was calculated by the blank+3x (Standard deviation of blank). At least three independent tests were performed (n≥3) for GlycoGrip.

FIG. 31 is a series of graphs showing the biolayer interferometry (BLI) sensogram of the ACE2 binding to variant of SARS-CoV-2 spike proteins.

FIG. 32 is a series of graphs showing the biolayer interferometry (BLI) sensogram of the heparin binding to variant of SARS-CoV-2 spike proteins.

FIG. 33 Panel A is a graph showing the Accessible Surface Area (ASA) plotted for 19 distinctive GAG hotspots on the SARS-CoV-2 spike protein in the closed-state calculated with a probe radius of 7.2 Å, calculated according to the Shrake-Rupley algorithm. Panel B is a graph showing the Accessible Surface Area (ASA) plotted for 19 distinctive GAG hotspots on the SARS-CoV-2 spike protein in the 1-up state calculated with a probe radius of 7.2 Å, calculated according to the Shrake-Rupley algorithm.

FIG. 34 shows both test strips and a corresponding graph of test strip signal intensities showing the dose-dependency results of Omicron detection using GlycoGrip2.0 without signal enhancement. The limit of the detection was calculated by the blank+3x (Standard deviation of blank). At least three independent tests were performed (n≥3) for Glycogrip.

FIG. 35 is a molecular representation of GAGs: hep2mer, hep4mer, h6s2mer, h6s4mer.

FIG. 36 shows a wide range of working temperature of the GlycoGrip. GlycoGrip was tested under 25° C., 40° C., and 50° C. There was no significant decrease in signal intensities up to 50° C. indicating the robustness of the GlycoGrip. At least three independent tests were performed (n≥3) for Glycogrip.

FIG. 37 shows test strips and a corresponding graph of test strip signal intensities showing the detection of the SARS-CoV-2 using monomeric ACE2.

FIG. 38 shows the detection of the SARS-CoV-2 in solution. GAGs (e.g., heparin) were conjugated to a gold nanoparticle (AuNP). ACE2 or NTD was conjugated to a magnetic nanoparticle. When the target is present, a clear solution was observed; while the control showed a colored solution. Panel A shows a graph of the optical spectrum measured by UV/VIS spectrophotometer and panel B shows a graph of the optical density at 530 nm.

FIG. 39 shows the detection of the SARS-CoV-2 on a gold substrate. GAGs (e.g., heparin or heparan sulfate) were conjugated to the gold surface and SAR-CoV-2 was optically detected. Panel A is a graph showing the optical response of the SARS-CoV-2 detection using heparin or heparan sulfate. Panel B shows a graph of the corresponding saturation curve.

FIG. 40 shows illustrations of signal enhancements methods according to some embodiments of the present invention. Panel A is an illustration of an exemplary signal enhancement by enzymatic reaction. Panel B is an illustration of an exemplary signal enhancement by chemical catalytic reaction.

FIG. 41 Panel A is a graphical illustration of the HIV-1 interaction between host cell with GAGs, CD4, and co-receptors (CXCR4 or CCR5). Panel B is a graph that shows the binding affinities between Env (monomeric gp140; Clade CRF_BC07) and HS/HEP (Steady-state analysis of BLI data to determine K_{D} values). Panel C is a schematic illustrating the GAGs based lateral flow assay (LF) biosensor for HIV detection. Panel D shows both test strips and a corresponding graph of test strip signal intensities showing the comparison of the signal intensity between HS and HEP for Env protein detection on LF biosensor. p value<0.05 (*) was determined using an unpaired two tailed t-test.

FIG. 42 Panel A shows both test strips and a corresponding graph of test strip signal intensities showing the selectivity of GAGs based LF biosensor was tested with various counter target proteins including SARS-CoV-2, SARS-CoV-1, MERS-CoV, human serum albumin, and human serum. Panel B shows both test strips and a corresponding graph of test strip signal intensities showing the dose-dependency of the GAGs based LF biosensor for HIV-1 Env protein with silver enhancement methods using different concentration of the Env protein in buffer condition. Limit of the detection (LOD) was 5 ng per reaction. Panel C shows both test strips and a corresponding graph of test strip signal intensities showing the dose-dependency of the GAGs based LF biosensor for HIV-1 Env protein with silver enhancement methods using different concentration of the Env protein in human serum condition. Limit of the detection (LOD) was 5 ng per reaction. Panel D is a graph that shows the stability of the GAGs based LF biosensor at 40° C. for 28 days. Three independent strips were tested to check the signal intensity change over time. p values<0.05 (*), 0.01 (**) and 0.001 (***) determined using a one-way ANOVA with Tukey's post hoc test.

FIG. 43 Panel A is a graph that shows the binding affinities between trimeric and monomeric Enc (Clade C) and HS (Steady-state analysis of BLI data to determine KD values). Panel B shows both test strips and a corresponding graph of test strip signal intensities showing the dose-dependency of the GAGs based LF biosensor for HIV-1 trimeric Env protein with silver enhancement methods using different concentration of the trimeric Env protein in buffer condition.

FIG. 44 Panel A is a graph that shows the binding affinities between variants of Env (trimeric gp140; Clade C, B, A) and HS (Steady-state analysis of BLI data to determine KD values). Panel B is a graph that shows the binding of HIV-1 trimeric Env protein to HS as measured by ELISA and an illustration showing the process of binding of HS to trimeric Env. Panel C is a graph that shows the binding affinities between variants of Env (trimeric gp140; Clade C, B, A) and CD4 (Steady-state analysis of BLI data to determine KD values) Panel D is a graph that shows the binding of HIV-1 trimeric Env protein to CD4 as measured by ELISA and an illustration showing the process of binding of CD4 to trimeric Env.

FIG. 45 shows both test strips and a corresponding graph of test strip signal intensities showing the detection of variants of HIV-1 trimeric Env protein using GAGs based LF biosensor for HIV-1 Env protein

### DETAILED DESCRIPTION OF EXAMPLE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

Like numbers refer to like elements throughout. In the figures, the thickness of certain lines, layers, components, elements or features may be exaggerated for clarity. The abbreviations “FIG. and “Fig.” for the word “Figure” can be used interchangeably in the text and figures.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, in “direct contact” with, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of an element or fabric in use or operation in addition to the orientation depicted in the figures. For example, if the fabric in the figures is inverted, elements described as “under” or “beneath” other elements or features would then be oriented “over” the other elements or features. Thus, the exemplary term “under” can encompass both an orientation of “over” and “under.” The fabric may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

Provided according to embodiments of the present invention are articles including a glycosaminoglycan (GAG). In some embodiments, an article of the present invention is a test strip. A “test strip” as used herein refers to an article used in testing a sample, the article having any suitable dimensions and which may or may not be in the form of strip. For example, a test strip may be square, rectangular, circular, etc. in shape. In some embodiments, a test strip of the preset invention is rectangular and/or includes a strip of material (e.g., includes a substrate that is in the form of a strip and/or a substrate that is rectangular in shape).

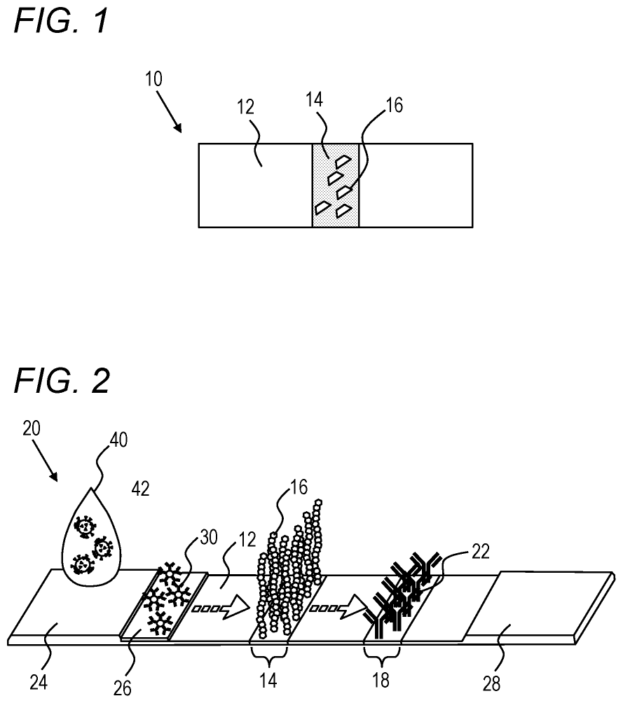

Referring to FIG. 1, a test strip 10 may comprise a substrate 12 including a test region 14, wherein the test region 14 comprises a glycosaminoglycan (GAG) 16 (e.g., a heparan sulfate proteoglycan (HSPG)) attached to the substrate 12. One or more (e.g., 1, 2, 3, 4, 5, 6, or more) different glycosaminoglycan(s) 16 may be attached to the substrate 12 and/or present at the test region 14. In some embodiments, two or more GAGs 16 are attached to the substrate 12 and/or present at the test region 14. For example, one (e.g., a single) test region 14 may include a mixture of heparin and heparan sulfate. In some embodiments, one test region 14 includes two or more GAGs 16 that are present at the test region 14 in the same amount or in different amounts. In some embodiments, a substrate 12 includes two or more GAGs 16 that are each present at a different test region 14; for example, a first test region 14 may include heparin and a second test region 14 may include heparan sulfate. A GAG 16 may also be referred to herein as a “capture probe” or “capture agent.” Exemplary glycosaminoglycans 16 include, but are not limited to, heparin, heparan sulfate (e.g., heparan sulfate proteoglycan (HSPG)), chondroitin sulfate, dextran sulfate, and/or any combination and/or fragment thereof. In some embodiments, the GAG 16 is a heparan sulfate proteoglycan. In some embodiments, the GAG 16 is heparin. In some embodiments, the GAG 16 is heparan sulfate. A GAG 16 of the present invention may have a molecular weight in a range of about 1, 5, or 10 kiloDaltons (kDa) to about 15, 20, 25, 30, 40, 50, 60, 70, 80, 90, 100, 110, 120, 130, 140, or 150 kDa. In some embodiments, the GAG 16 has a molecular weight of about 1, 5, 10, 15, 20, 25, 30, 35, 40, 45, 50, 55, 60, 65, 70, 75, 80, 85, 90, 95, 100, 110, 120, 130, 140, or 150 kDa. In some embodiments, the GAG 16 has a molecular weight of about 10 kDa to about 100 kDa. In some embodiments, the GAG 16 has a molecular weight of about 10 kDa to about 50 kDa. In some embodiments, the GAG 16 has a molecular weight of about 1 kDa to about 10 kDa. In some embodiments, the GAG 16 has a molecular weight of about 15 kDa. A GAG 16 of the present invention may be a synthetic GAG (i.e., a GAG that is synthetically produced) or may be from any suitable source and/or tissue. In some embodiments, a GAG 16 is synthetically produced (e.g., chemically synthesized). In some embodiments, a GAG 16 is obtained from a natural source. In some embodiments, the GAG 16 is from a mammal such as a bovine, porcine, or human and/or from the liver, kidney, mucosa, lung, and/or muscle of the mammal.

As shown in FIG. 1, a test strip 10 of the present invention comprises a substrate 12 including a test region 14, wherein the test region 14 comprises a GAG 16. A test region 14 may be referred to interchangeably as a test line 14. The GAG 16 may be attached (e.g., covalently and/or noncovalently) to a portion of the substrate 12 and/or test region 14. In some embodiments, a GAG 16 is adsorbed (e.g., non-specifically adsorbed) onto and/or into at least a portion of the substrate 12 and/or test region 14. For example, in some embodiments, a GAG 16 may be bound (e.g., covalently and/or non-covalently) to one half of a binding pair and the substrate 12 may be bound (e.g., covalently and/or non-covalently) to the other half of the binding pair and the two halves of the binding pair are bound (e.g., covalently and/or non-covalently) such that the GAG 16 is bound (e.g., non-specifically adsorbed) to the substrate 12 through the binding pair. In some embodiments, a GAG 16 may be attached to (e.g., conjugated to) biotin and the substrate 12 may include streptavidin, optionally streptavidin adsorbed (e.g., non-specifically adsorbed such as through electrostatic and/or hydrophobic interaction(s)) onto and/or into a portion of the substrate 12, such that the GAG 16 that is attached to biotin can be bound onto and/or into the substrate 12 through the binding of biotin and streptavidin. In some embodiments, the test region 14 is a portion of the substrate 12 (e.g., an area and/or line on and/or in the substrate 12). The presence of the GAG 16 on and/or in the substrate 12 may define the test region 14 (e.g., the area of the test region 14). One or more (e.g., 1, 2, 3, 4, 5, 6, or more) test region(s) 14 may be present on the substrate 12. When the substrate 12 includes two or more test regions 14, the two or more test regions 14 may be separated from each other. In some embodiments, one test region 14 of the two or more test regions 14 may include the same GAG 16 or the same mixture of GAGs 16 as another test region 14 of the two or more test regions 14 and/or one test region 14 of the two or more test regions 14 may include different GAGs and/or a different mixture of GAGs 16 than another test region 14 of the two or more test regions 14. When the substrate 12 includes two or more test regions 14 and at least one test region 14 of the two or more test regions 14 includes a different GAG and/or a different mixture of GAGs 16 than another test region 14 on the substrate 12, then two or more (e.g., 2, 3, 4, 5, 6, 7, or more) target agents 42 may detected using a single test strip 10. In some embodiments, the GAG 16 comprises heparin, optionally heparin having a molecular weight in a range of about 10 kDa to about 20 kDa and/or heparin that is synthetic heparin or obtained from a mammal (e.g., a porcine). In some embodiments, the GAG 16 comprises heparan sulfate, optionally heparan sulfate having a molecular weight in a range of about 10 kDa to about 20 kDa and/or heparan sulfate that is synthetic heparan sulfate or obtained from a mammal (e.g., a bovine). A GAG 16 may be present on a substrate 12 and/or test region 14 in an amount of about 1 mg/mL to about 5 mg/mL (e.g., about 1, 1.5, 2, 2.5, 3, 3.5, 4, 4.5, or 5 mg/mL).

Exemplary substrates 12 include, but are not limited to, nitrocellulose, cellulose, metals (gold, silver, etc.), glass, chromatography paper, and/or the like. In some embodiments, the substrate 12 is configured to move a fluid sample by capillary action through the substrate 12 such as through the substrate 12 to the test region 14 and/or an optional control region 18. A control region 18 may be referred to interchangeably as a control line 18. As shown, for example, in FIG. 2, a test strip 20 of the present invention may include a control region 18. A control region 18 may comprise a control agent 22 (e.g., an anti-IgG antibody) attached (e.g., covalently and/or non-covalently) to a portion of the substrate 12. In some embodiments, a control agent 22 is adsorbed (e.g., non-specifically adsorbed) onto and/or into a portion of the substrate 12. The presence of control agent 22 on and/or in the substrate 12 may define the control region 18 (e.g., the area of the control region 18). The substrate 12 may be configured to move a fluid sample by capillary action through the substrate 12 to the control region 18.

The test strip 20 may include a sample pad 24, a conjugate pad 26, and/or an adsorption pad 28. The sample pad 24 may be in contact (directly or indirectly) with the substrate 12 and the sample pad 24 may be configured to be contacted by a fluid sample 40 and/or may be configured to move the fluid sample 40 by capillary action through the sample pad 24 to the substrate 12. In some embodiments, the sample pad 24 comprises an absorbent material such as cellulose (e.g., nitrocellulose). The sample pad 24 may aid in controlling the rate and/or amount of fluid that contacts the substrate 12.

In some embodiments, the test strip 20 is devoid of a conjugate pad 26 (also referred to herein as a conjugation pad). In some embodiments, the test strip 20 comprises a conjugate pad 26. The conjugate pad 26 may include detection agent 30. In some embodiments, the detection agent 30 may be present in (e.g., embedded in) and/or on the conjugate pad 26. The detection agent 30 may comprise a protein (e.g., an antibody), a peptide, a polysaccharide (e.g., a GAG), a nucleic acid, and/or any combination and/or fragment thereof. The detection agent 30 may comprise an antibody and/or a fragment thereof attached to (e.g., coupled to) a signaling agent. When the detection agent 30 comprises a GAG, the GAG of detection agent 30 may be the same as or different than GAG 16. The conjugate pad 26 may contact (directly or indirectly) the sample pad 24 and may contact (directly or indirectly) the substrate 12 and as such a fluid (e.g., a fluid sample 40) may be absorbed by the conjugate pad 26 from the sample pad 24 and the fluid may then be adsorbed by the substrate 12 from the conjugate pad 26. The conjugate pad 26 may comprise an absorbent material and/or glass fibers and the detection agent 30 may be within and/or on a surface of the conjugate pad 26.

An adsorption pad 28 may contact (directly or indirectly) the substrate 12. In some embodiments, an adsorption pad 28 may be included at an end of the test strip 20 that is opposite the end at which the fluid sample 40 is contacted to the substrate 12 and/or opposite the end at which the sample pad 24 is present. In some embodiments, the adsorption pad 28 comprises an absorbent material such as cellulose (e.g., nitrocellulose). The adsorption pad 28 may absorb fluid from the substrate 12. In some embodiments, the test strip 20 is a lateral flow test strip, wherein the test strip 20 is configured to move a fluid sample by capillary action through the sample pad 24 to the conjugate pad 26, substrate 12, test region 14, control region 18, and/or adsorption pad 28. In some embodiments, the test strip 20 is not a lateral flow test strip and the test strip 20 is not configured to move a fluid sample by capillary action through the substrate 12. In some embodiments, the test strip 20 does not include (i.e., is devoid of) a sample pad 24, conjugate pad 26, and/or adsorption pad 28. In some embodiments, the test strip 20 comprises a substrate 12 (e.g., a metal or glass substrate) that does not absorb the fluid sample and the test strip 20 optionally comprises a sample pad 24, conjugate pad 26, and/or adsorption pad 28. In some embodiments, the test strip 20 comprises a substrate 12 (e.g., a metal or glass substrate) that does not absorb the fluid sample and the fluid sample may contact a test region 14 and optional control region 18 that is/are present on a surface of the substrate 12.

A “detection agent” as used herein refers to compound that can bind (e.g., covalently and/or non-covalently) and/or is configured to bind to a target agent 42. In some embodiments, a detection agent 30 can bind to a target agent 42 and can be detected (e.g., optically, visually, electronically, electrochemically, and/or chemically). In some embodiments, a detection agent 30 comprises a polypeptide (e.g., a protein such as an antibody or a portion thereof or a peptide), a polysaccharide (e.g., a GAG), a nucleic acid, and/or any combination and/or fragment thereof that is bound to (e.g., covalently and/or non-covalently) a signaling agent. A “signaling agent” as used herein refers to a compound that can be detected such as detected optically, visually (e.g., by the human eye), electronically, electrochemically, and/or chemically). In some embodiments, signal intensity may be measured using method known to those of skill in the art. Exemplary signaling agents include, but are not limited to, a metal particle (e.g., a metal nanoparticle, metal nanorods) such as a gold particle (e.g., a gold nanoparticle) and/or a silver nanoparticle, a latex particle (e.g., a latex microsphere), a silica particle (e.g., a silica nanoparticle), a quantum dot, a nanodiamond, a magnetic particle (e.g., a magnetic nanoparticle and/or magnetic bead), an upconversion nanoparticle, a fluorescent dye, a nanocellulose bead, an organic nanomaterial (e.g., a carbon dot and/or carbon nanotube), an organic-metallic material (e.g., a metal-organic framework), and/or any combination thereof. In some embodiments, the detection agent 30 includes a biological compound such as, but not limited to, a polypeptide (e.g., an affimer), aptamer, peptide, nanobody, and/or the like that can bind and/or be configured to bind a target agent 42. In some embodiments, a detection agent 30 comprises an antibody labeled gold nanoparticle such as a spike specific monoclonal antibody labeled gold nanoparticle. A detection agent 30 may be contacted to (e.g., deposited onto) a test strip of the present invention and/or may be present in a test strip of the present invention, upon contact with a fluid sample 40, in a volume of about 1, 5, 10, 15, or 20 μL to about 25, 30, 35, 40, 45, or 50 μL and/or at a concentration of about 1, 5, 10, 15, or 20 nM to about 25, 30, 35, 40, 45, or 50 nM.

The detection agent 30 can bind and/or is configured to bind a target agent 42, which may be a pathogen (e.g., a virus). A detection agent 30 may also be referred to herein as a “signaling probe.” In some embodiments, the pathogen is a virus (e.g., a virus with a glycoprotein) and/or the detection agent 30 can bind and/or is configured to bind a portion of a viral polypeptide. Exemplary viruses include, but are not limited to, a virus in the Coronaviridae family such as severe acute respiratory coronavirus 2 (SARS-CoV-2) or a variant thereof and/or human immunodeficiency virus (HIV). In some embodiments, the virus is a variant of SARS-CoV-2 such as, but not limited to, SARS-CoV-2 Alpha strain, SARS-CoV-2 Beta strain, SARS-CoV-2 Gamma strain, SARS-CoV-2 Delta strain, SARS-CoV-2 Omicron strain, and/or a variant thereof (e.g., an emerging variant). In some embodiments, the virus is a beta-coronavirus and/or the detection agent 30 can bind and/or is configured to bind a viral polypeptide of a beta-coronavirus. In some embodiments, the detection agent 30 can bind and/or is configured to bind a viral spike protein, such as a beta-coronavirus spike protein (e.g., a SARS-CoV spike and/or MERS-CoV spike). In some embodiments, the detection agent 30 comprises a REGN10933 (e.g., having a sequence as provided at Protein Data Bank ID: EMD-22137), an angiotensin converting enzyme 2 (ACE2) (e.g., an ACE2 having a sequence as provided at www.uniprot.org/uniprot/Q9BYF1-1 and/or a human monomeric and/or dimeric ACE2, optionally human ACE2 that is commercially available from Acro Biosystems of Newark, Delaware as Human ACE2, Fc Tag (AC2-H5255) that is expressed from human 293 cells), a NTD antibody, and/or a fragment thereof. Further exemplary antibodies include, but are not limited to, those listed in Table 1 and/or a fragment thereof.

TABLE 1
Exemplary antibodies that may be used in a detection agent 30.

| | Commercially | Product |
|---|---|---|
| Antibodies | available from | Number |
| Anti-SARS-CoV-2 Spike NTD (clone 2215) | Leinco Technologies, Inc. | LT6000 |
| Anti-SARS-CoV-2 Spike NTD (clone 2146) | Leinco Technologies, Inc. | LT2000 |
| SARS-CoV-2 (2019-nCoV) Spike | Sinobiological | 40591-MM43 |
| Neutralizing Antibody, Mouse Mab | | |
| SARS-CoV-2 (2019-nCoV) Spike | Sinobiological | 40592-MM57 |
| Neutralizing Antibody, Mouse Mab | | |
| SARS-CoV-2 (2019-nCoV) Spike | Sinobiological | 40592-R001 |
| Neutralizing Antibody, Rabbit Mab | | |
| SARS-CoV/SARS-CoV-2 Spike | Sinobiological | 40150-D001 |
| antibody, Chimeric Mab | | |
| SARS-CoV/SARS-CoV-2 Spike | Sinobiological | 40150-D002 |
| antibody, Chimeric Mab | | |
| SARS-CoV-2 (2019-nCoV) Spike | Sinobiological | 40591-MM43 |
| Neutralizing Antibody, Mouse Mab | | |
| SARS-CoV-2 (2019-nCoV) Spike | Sinobiological | 40592-R118 |
| Neutralizing Antibody, Rabbit Mab | | |
| SARS-CoV-2 (2019-nCoV) Spike | Sinobiological | 40592-R117 |
| Neutralizing Antibody, Rabbit Mab | | |
| SARS-CoV-2 (2019-nCoV) Spike S1 | Sinobiological | 40150-R007 |
| Antibody, Rabbit Mab | | |
| SARS-CoV-2 Spike Antibody, Omicron | Sinobiological | 40592-MM117 |
| Reactive, Mouse Mab | | |
| Coronavirus Spike Protein Monoclonal | Invitrogen | MA5-29970 |
| Antibody (7) | | |
| SARS-CoV-2 Spike Protein S1 Chimeric | Invitrogen | MA5-35939 |
| Recombinant Human Monoclonal Antibody (H6) | | |
| Coronavirus Spike Protein Recombinant | Invitrogen | MA5-29971 |
| Rabbit Monoclonal Antibody (28) | | |
| SARS-CoV-2 Spike Protein S1 Monoclonal | Invitrogen | MA5-36247 |
| Antibody (HL6) | | |
| Recombinant Anti-SARS-CoV-2 Spike | abcam | ab273073 |
| Glycoprotein S1 antibody [CR3022] (ab273073) | | |
| Anti-SARS-CoV-2 Spike Ectodomain | abcam | ab277512 |
| antibody [CV1] | | |
| Anti-SARS-CoV-2 spike glycoprotein | abcam | ab272504 |
| antibody - Coronavirus | | |
| SARS-CoV-2 Spike S1 Subunit Antibody | R&D systems | MAB105403 |
| SARS-CoV-2 Spike S1 Subunit Antibody | R&D systems | MAB105407 |
| SARS-CoV-2 Spike S1 Subunit Antibody | R&D systems | MAB105405 |
| SARS-CoV-2 Spike S1 Subunit Antibody | R&D systems | MAB105805 |
| SARS-CoV-2 Spike S1 Subunit Antibody | R&D systems | AF10770 |
| SARS-CoV-2 Spike S1 Subunit Antibody | R&D systems | MAB10637 |
| Anti-SARS-CoV-2 Spike S1 Antibody, | ACRObiosystems | S1N-S58A1-1MG |
| Mouse IgG1 (AS58) | | |
| Anti-SARS-CoV-2 Spike S1 Antibody, | ACRObiosystems | S1N-S59A1-100UG |
| Mouse IgG1 (AS59) | | |
| Mouse Anti-SARS-CoV-2 Antibody | MyBioSource.com | MBS355887 |
| SARS-CoV-2 Spike RBD Nanobody | EpiGentech | A73680 |
| Rabbit anti-SARS-CoV-2 S1RBD | RayBiotech | 130-10784-100 |

In some embodiments, a detection agent 30 comprises two or more different compounds selected from: a polypeptide (e.g., an antibody or a portion thereof or a peptide), a polysaccharide (e.g., a GAG), and a nucleic acid, wherein the two or more different compounds may optionally be attached to the same signaling agent. In some embodiments, a detection agent 30 comprises two or more different antibodies and/or fragments thereof that are optionally attached to the same signaling agent. In some embodiments, the detection agent 30 comprises a NTD antibody and/or a fragment thereof that is optionally attached to a signaling agent (e.g., a gold nanoparticle). In some embodiments, the detection agent 30 comprises an ACE2 (e.g., a human ACE2) and/or a fragment thereof that is optionally attached to a signaling agent (e.g., a gold nanoparticle). In some embodiments a monomeric and/or dimeric ACE2 (e.g., a human ACE2) that is optionally attached to a signaling agent (e.g., a gold nanoparticle) is used to bind and/or is configured to bind a variant of SARS-CoV-2 such as, SARS-CoV-2 Omicron strain and/or a variant thereof (e.g., an emerging variant). FIG. 37 shows the signal intensity and signals from a detection agent comprising a monomeric ACE2 (commercially available from Sinobiological; Cat #10108-H08H-100) that included a signaling agent that was used to detect SARS-CoV-2.

The GAG 16 may have a binding affinity for the target agent 42 (e.g., pathogen) of about 215 nM K_{D} or less. In some embodiments, the GAG 16 has a binding affinity for the target agent 42 of about 215, 200, 175, 150, 125, 100, 75, 50, 25, 10, or 5 nM K_{D} or less. In some embodiments, the GAG 16 has a binding affinity for the target agent 42 of about 1, 5, 10, 15, 20, or 25 nM K_{D} to about 30, 40, 50, 60, 70, 80, 90, 100, 150, or 200 K_{D}. In some embodiments, the GAG 16 has a binding affinity for the target agent 42 of 10 nM K_{D} to about 25 nM K_{D}.

A fluid sample 40 may be contacted to a test strip of the present invention (e.g., test strip 10 and/or test strip 20) in any suitable manner. In some embodiments, a fluid sample 40 is deposited onto the test strip and/or added to the test strip. In some embodiments, all or a portion of a test strip of the present invention is submerged in and/or dipped in the fluid sample 40. As shown, for example, by the arrows in FIG. 2, a fluid sample 40 optionally including a target agent 42 may be contacted to a sample pad 24 and the fluid sample 40 may move through the sample pad 24, optionally by capillary action, to the optional conjugation pad 26 and to the substrate 12 including the test region 14 and/or optional control region 18. When a conjugation pad 26 is present, the fluid sample 40, upon contacting the conjugation pad 26, may include the detection agent 30, and the fluid sample 40 including the detection agent 30 may move to the substrate 12. Alternatively, or in addition, the detection agent 30 may be added to the fluid sample 40 prior to contacting the fluid sample 40 and the test strip. The detection agent 30 may bind to the target agent 42 if present in the fluid sample 40. Upon contact of the fluid sample 40 and the test region 14, if the target agent 42 is present in fluid sample 40, then the target agent 42 may bind to the GAG 16 at the test region 14 and the detection agent 30 may bind to or be bound to the target agent 42. A signal (e.g., a color, fluorescence, and/or the like) may be provided at the test region 14 when the target agent 42 is bound to the GAG 16 and the detection agent 30 is bound to the target agent 42. If a control region 18 is present, then upon contact of the fluid sample 40 and the control region 18, the control agent 22 may bind the detection agent 30 and another signal (e.g., a color, fluorescence, and/or the like) may be provided at the control region 18 when the detection agent 30 is bound to the control agent 22.

A signal provided at the test region 14 and a signal provided at the control region 18 may be the same (e.g., the same color) or may be different (e.g., different colors). In some embodiments, a signal provided at the test region 14 and a signal provided at the control region 18 may have different shades, intensities, and/or brightness. A signal provided at the test region 14 and/or control region 18 may be increased using methods known in the art such as, but not limited to, using a reporter system (e.g., horseradish peroxidase) that can increase the strength (e.g., degree of color, intensity, brightness, and/or the like) of the signal. A signal provided at the test region 14 and a signal provided at the control region 18 may appear at the same time or at different times and may both occur together over a period of time.

A test strip of the present invention (e.g., test strip 10 and/or test strip 20) may have a sensitivity and/or time to detection (e.g., a signal at the test region 14 and/or control region 18) of about 30 minutes or less, responsive to contacting a fluid sample to the substrate and/or sample pad of the test strip. For example, upon contacting the fluid sample to the substrate and/or sample pad of the test strip with the initial contact being the starting time point (i.e., t=0), a signal (e.g., a signal from and/or produced by a detection agent 30) may be provided on and/or by the test strip within about 30 minutes or less after initial contact. In some embodiments, a signal (e.g., a signal from and/or produced by a detection agent 30) is provided on and/or by the test strip at about 1, 2, 3, 4, 5, 6, 7, 8, 9, or 10 minute(s) to about 11, 12, 13, 14, 15, 16, 17, 18, 19, 20, 21, 22, 23, 24, 25, 26, 27, 28, 29, or 30 minutes after initial contact. In some embodiments, the signal is provided at about 1, 2, 5, 10, 15, 20, or 25 minutes after initial contact of the fluid sample to the substrate and/or sample pad. In some embodiments, a signal is provided at a test region and/or control region of a test strip at about 1, 2, 3, 4, 5, 6, 7, 8, 9, or 10 minute(s) to about 11, 12, 13, 14, 15, 16, 17, 18, 19, 20, 21, 22, 23, 24, 25, 26, 27, 28, 29, or 30 minutes after initial contact of the fluid sample to the substrate and/or sample pad.

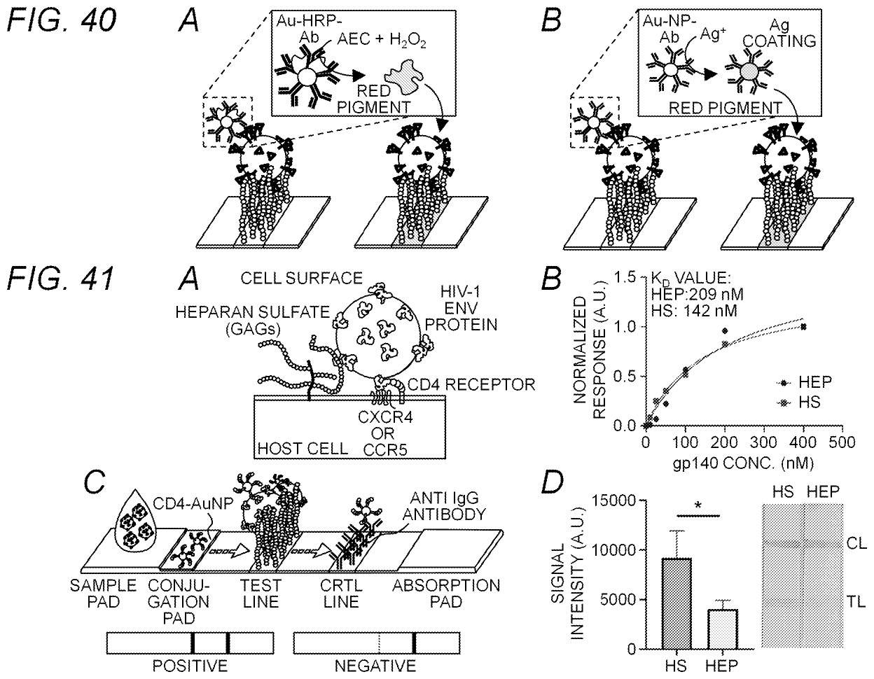

In some embodiments, a test strip of the present invention (e.g., test strip 10 and/or test strip 20), binding pair of the present invention, and/or method of the present invention may utilize a reporter system (e.g., a signal enhancement method) to enhance the limit of detection for a target agent 42. Exemplary reporter systems and/or signal enhancement methods may include, but are not limited to, catalytic reactions (enzymatic or chemical), metal precipitations, optical enhancements (e.g., surface enhanced raman), Rolling circle amplification, isothermal DNA amplification, catalytic nanoparticles, DNAzymes, and any combinations thereof. In some embodiments, a catalytic reaction is used to enhance a signal and the catalytic reaction substrate may be, but is not limited to, AEC (3-amino-9-ethylcarbazole), TMB (3,3′, 5,5″-tetramethylbenzidine), ABTS (2,2′-azino-bis [3-ethylbenzthiazoline-6-sulfonic acid]). FIG. 40 shows exemplary illustrations of signal enhancement methods. As shown in panel A of FIG. 40, a detection agent included a signaling probe along with an enzyme (e.g., horseradish peroxidase) and addition of the enzymatic reaction substrate solution (e.g., 100 μl AEC and/or AEC staining kit optionally commercially available Sigma-Aldrich; Cat #AEC101) produces a red pigment that enhances the color on the test line of the test strip. As shown in panel B of FIG. 40, a detection agent included a gold nanoparticle and a solution of silver ions (e.g., 0.3% w/w) and a reducing agent (e.g., 3% w/w hydroquinone) was added to the test strip such that the silver ion was reduced on the gold nanoparticle surface resulting in a black color on the test line of the test strip.

In some embodiments, a test strip of the present invention (e.g., test strip 10 and/or test strip 20) may have a limit of detection for a target agent 42 (e.g., a pathogen) of about 80 ng/reaction or less such as about 75, 60, 50, 40, 30, or 20 ng/reaction or less. In some embodiments, the test strip has a limit of detection for a target agent 42 of about 1, 5, 10, 15, 20, 25, 30, 35, 40, 45, 50, 55, 60, 65, or 70 ng/reaction. In some embodiments, the test strip has a limit of detection for a target agent 42 of about 20 ng/reaction or less. In some embodiments, the test strip has a limit of detection for a target agent 42 of about 1, 2, 3, 4, 5, 6, 7, 8, 9, 10, 11, 12, 13, 14, 15, 16, 17, 18, 19, or 20 ng/reaction.

A test strip of the present invention (e.g., test strip 10 and/or test strip 20) and/or a binding pair of the present invention may be shelf-stable for at least 30, 40, 45, or 50 days or more. “Shelf-stable” as used herein in reference to a test strip or binding pair refers to the test strip or binding pair, respectively, upon being stored under recommended storage conditions, having no or minimal (e.g., about 20% or less) decrease in signal intensity compared to the signal intensity of the test strip or binding pair at the time of initial formation and/or at day 1 after formation. In some embodiments, a test strip and/or binding pair of the present invention may be stored at about 20° C. to about 30° C., 35° C., 40° C., 45° C., 50° C., 55° C., or 60° C. (optionally at about 25° C.) in a vinyl and/or plastic container (e.g., a vinyl and/or plastic bag), optionally with a desiccant (e.g., a silica desiccant). In some embodiments, a test strip and/or binding pair of the present invention may have a working temperature at about 20° C. to about 30° C., 35° C., 40° C., 45° C., 50° C., 55° C., or 60° C. In some embodiments, a test strip and/or binding pair of the present invention has a working temperature of greater than 30° C. (e.g., of at least 31° C., 32° C., 33° C., 34° C., 35° C., 36° C., 37° C., 38° C., 39° C., 40° C., 45° C., 50° C., 55° C., or 60° C.). “Working temperature” as used herein refers to the temperature at which a test strip or binding pair is able to detect a target (e.g., a target agent 42) when present in a sample in an amount at or above the limit of detection. For example, a test strip and/or binding pair in use (e.g., from the time when the first member and second member are combined with a fluid sample until detection of a signal and/or the complex) may be carried out at a temperature. In some embodiments, a test strip and/or binding pair of the present invention has a signal intensity and/or limit of detection that is within +20% or less (e.g., within +5%, 10%, or 15%) over a temperature at about 20° C. to about 30° C., 35° C., 40° C., 45° C., 50° C., 55° C., or 60° C. In some embodiments, a test strip and/or binding pair of the present invention has no decrease or a decrease of less than 20% (e.g., less than 5% or 10%) in signal intensity when used at two or more different temperatures in a range of about 20° C. to about 30° C., 35° C., 40° C., 45° C., 50° C., 55° C., or 60° C., optionally in a range of about 25° C. to about 50° C. FIG. 36 shows images of exemplary test strips and a graph that demonstrates no significant decrease in signal intensity for test strips tested at 25° C., 40° C., and 50° C.

A test strip of the present invention (e.g., test strip 10 and/or test strip 20) may be prepared by contacting a GAG 16 onto at least a portion of a surface of a substrate 12. The GAG 16 may be attached (e.g., covalently or noncovalently) to a portion of the substrate 12. For example, a GAG 16 may be attached to substrate 12 that is a nitrocellulose (NC) membrane and the GAG 16 may be attached to the NC membrane through the adsorption of one half of a binding pair (e.g., streptavidin) that is bound to the NC membrane. Streptavidin can non-specifically adsorb to the NC membrane through electrostatic interaction and hydrophobic interaction, and a GAG 16 attached to the other half of a binding pair (e.g., biotin modified GAGs) may then be conjugated to the streptavidin. In some embodiments, a GAG 16 may be contacted to (e.g., dispensed onto, added to, and/or the like) a portion of the substrate 12 and the substrate 12 following contact with the GAG 16 may be dried for a period of time, optionally the substrate 12 comprising the GAG 16 may be dried at about 20° C. to about 70° C. for about 5, 10, 15, or 20 minutes to about 1, 2, 6, 12, 18, 20, 24, 28, or 30 hours. In some embodiments, the substrate 12 comprising the GAG 16 may be dried at about 55° C. for about 20 min, at about 37° C. for about 1 hour, and/or at about 25° C. for 24 hours. In some embodiments, a composition comprising a GAG 16 may be contacted to a substrate 12 and the composition may be configured to aid in binding of the GAG 16 to the substrate 12 (e.g., NC membrane). For example, the components present in the composition and/or the pH of the composition may be designed and/or configured to decrease the electrostatic repulsion between GAG 16 and the substrate 12. In some embodiments, the pH of the composition may be equal to or below the isoelectric point of streptavidin and/or the composition may have an ionic strength that decreases the electrostatic repulsion between the GAG 16 and the substrate. The composition may comprise a GAG 16 in an amount of about 0.01, 0.05, 0.1, 0.5, or 1 mg/mL to about 1.25, 1.5, 1.75, 2, 2.5, 3, 3.5, 4, 4.5, or 5 mg/mL.

In some embodiments, provided is a glycosaminoglycan (GAG) that is optionally attached to a substrate and/or to a signaling agent. In some embodiments, the GAG is attached to a substrate (e.g., a metal and/or glass) as described herein and when a composition optionally comprising a target (e.g., a pathogen) is contacted to the substrate, the substrate may be immobile in the composition. In some embodiments, a GAG may be attached to a substrate and may remain attached to the substrate following contact with a composition (e.g., an aqueous composition optionally comprising a target). In some embodiments, the GAG is mobile in a composition (e.g., free in solution and/or suspended in the composition) that optionally comprises a target (e.g., a pathogen) and the GAG may be attached to a signaling agent as described herein. A method of detecting a target (e.g., a pathogen) may comprise combining a GAG optionally attached to a substrate and/or signaling agent and detecting binding of the GAG to the target. Methods of detecting include those described herein such as optically, visually, electronically, electrochemically, and/or chemically detecting binding of the GAG and a target and/or detecting a signal (e.g., from a signaling agent and/or from a reporter system) and/or a signaling agent. Detecting may comprise measuring the color, signal intensity, size (e.g., particle and/or complex size) and/or another unit of measure of a signal or means to detect binding and/or the presence of the target. In some embodiments, surface plasmon resonance is used to detect a signal and/or presence of the target.

According to some embodiments, a binding pair is provided comprising a first member of the binding pair that includes a GAG 16 and a second member of the binding pair that includes a detection agent 30 that optionally comprises a signaling agent, wherein the first member and the second member are each configured to bind to the same target (e.g., target agent 42). In some embodiments, the first member comprises a signaling agent as described herein and the GAG 16 may be attached to (e.g., covalently and/or non-covalently) the signaling agent. A first member may also be referred to herein as a “capture probe” or capture agent. In some embodiments, at least one of the first member and the second member is devoid of a signaling agent. In some embodiments, the second member is devoid of a signaling agent. In some embodiments, the first member is devoid of a signaling agent.

The first member and second member may bind (e.g., covalently and/or non-covalently) and/or may be configured to bind to different portions and/or locations of the target. The target may be a target agent 42 as described herein. A binding pair of the present invention may be present as two separate (i.e., not bound) entities or may be present in the form of a complex comprising the first member and second member. In some embodiments, a first member is bound (e.g., covalently and/or non-covalently) to a first portion of a target and a second member is bound (e.g., covalently and/or non-covalently) to a second portion of the target, thereby providing a complex. In some embodiments, a binding pair may form a ternary complex and/or a sandwich complex when in the presence of a target to which the first member and second member bind.

A first member of a binding pair of the present invention comprises or is a GAG as described herein. For example, in some embodiments, a first member comprises heparin, heparan sulfate (e.g., heparan sulfate proteoglycan (HSPG)), chondroitin sulfate, and/or dextran sulfate, and/or a fragment thereof. In some embodiments, the GAG is attached to a signaling agent as described herein (e.g., a metal particle (e.g., a metal nanoparticle, metal nanorods) such as a gold particle (e.g., a gold nanoparticle) and/or a silver nanoparticle, a latex particle (e.g., a latex microsphere), a silica particle (e.g., a silica nanoparticle), a quantum dot, a nanodiamond, a magnetic particle (e.g., a magnetic nanoparticle and/or magnetic bead), an upconversion nanoparticle, a fluorescent dye, a nanocellulose bead, an organic nanomaterial (e.g., a carbon dot and/or carbon nanotube), an organic-metallic material (e.g., a metal-organic framework), and/or any combination thereof). In some embodiments, the first member of a binding pair comprises a GAG that is attached to a metal nanoparticle such as a gold nanoparticle.

A second member of a binding pair of the present invention comprises or is a detection agent as described herein. For example, in some embodiments, a second member comprises a protein (e.g., an antibody), a peptide, a polysaccharide (e.g., a GAG), a nucleic acid, and/or a fragment of any thereof that is optionally coupled to a signaling agent. In some embodiments, the second member is attached to (e.g., covalently and/or noncovalently) to a signaling agent. In some embodiments, the detection agent comprises an antibody that is coupled to a signaling agent as described herein. The signaling agent of a second member of a binding pair may be the same as or different than a signaling agent of a first member of the binding pair. In some embodiments, the detection agent comprises a GAG that is coupled to a signaling agent and the GAG of the detection agent may be the same as or different than the GAG of the first member.

A first member of a binding pair and/or a second member of a binding pair may have a binding affinity for the target of about 215 nM K_{D} or less. In some embodiments, the first member and/or second member has a binding affinity for the target of about 215, 200, 175, 150, 125, 100, 75, 50, 25, 10, or 5 nM K_{D} or less. In some embodiments, the first member and/or second member has a binding affinity for the target of about 1, 5, 10, 15, 20, or 25 nM K_{D} to about 30, 40, 50, 60, 70, 80, 90, 100, 150, or 200 K_{D}. In some embodiments, the first member and/or second member has a binding affinity for the target of 10 nM K_{D} to about 25 nM K_{D}.

In some embodiments, a binding pair of the present invention may have a limit of detection for a target (e.g., a pathogen) of about 80 ng/reaction or less such as about 75, 60, 50, 40, 30, or 20 ng/reaction or less. In some embodiments, the binding pair has a limit of detection for a target of about 1, 5, 10, 15, 20, 25, 30, 35, 40, 45, 50, 55, 60, 65, or 70 ng/reaction. In some embodiments, the binding pair has a limit of detection for a target of about 20 ng/reaction or less. In some embodiments, the binding pair has a limit of detection for a target of about 1, 2, 3, 4, 5, 6, 7, 8, 9, 10, 11, 12, 13, 14, 15, 16, 17, 18, 19, or 20 ng/reaction. In some embodiments, a binding pair of the present invention may be configured to bind a target in a composition having a pH about 6, 6.5, or 7 to about 7.5, or 8 and/or a salt concentration (e.g., NaCl) of about 1 or 5 mM to about 10, 20, 30, 40, 50, 60, 70, 80, 90, or 100 mM.

In some embodiments, a binding pair of the present invention may have a sensitivity and/or time to detection (e.g., a signal from the signaling agent) of about 30 minutes or less, responsive to combining the binding pair and the target. For example, upon contacting the first member, second member, and target (e.g., optionally in a fluid sample) with the initial contact being the starting time point (i.e., t=0), a signal (e.g., a signal from and/or produced by a detection agent 30) may be provided within about 30 minutes or less after initial contact. In some embodiments, a signal (e.g., a signal from and/or produced by a detection agent 30) is provided in about 1, 2, 3, 4, 5, 6, 7, 8, 9, or 10 minute(s) to about 11, 12, 13, 14, 15, 16, 17, 18, 19, 20, 21, 22, 23, 24, 25, 26, 27, 28, 29, or 30 minutes after initial contact. In some embodiments, the signal is provided in about 1, 2, 5, 10, 15, 20, or 25 minutes after initial contact (e.g., combination of the first member, second member, target).

A composition comprising a binding pair of the present invention may be provided. In some embodiments, the composition comprises a solvent such as, but not limited to, water. In some embodiments, the composition comprises an aqueous composition as described herein. The first member and/or the second member are free in solution in the composition. In some embodiments, a first member and/or second member is/are dissolved or suspended in a composition of the present invention. In some embodiments, a first member or a second member are bound to a substrate in a composition of the present invention and the substrate may be immobile in the composition. A first member and/or a second member may be in the form of a solid (e.g., a powder and/or a plurality of particles) or a liquid (e.g., suspended or dissolved in a liquid solvent) in a composition of the present invention.

According to some embodiments a kit may be provided. A kit of the present invention may comprise a binding pair of the present invention. The first member and the second member may be separately stored in the kit (e.g., in separate containers). In some embodiments, the first member and the second member are stored together (e.g., the same container and/or composition) in the kit. The first member and the second member may be in any suitable form in the kit. In some embodiments, the first member and/or the second member is/are in the form of a solid (e.g., a powder) or a liquid (e.g., suspended or dissolved in a liquid solvent).

In some embodiments, a kit may include a test strip of the present invention (e.g., test strip 10 and/or test strip 20) and a detection agent 30 configured to bind a target agent 42 (e.g., a pathogen). The test strip may be as provided in FIG. 1 or 2. In the kit, the detection agent 30 may be separate from the test strip (e.g., in the kit the test strip and detection agent are separated from one another and/or are separately packaged or stored). In some embodiments, the detection agent 30 is in the form of a powder or dry composition that is optionally separate from the test strip. In some embodiments, the detection agent 30 is a powder or dry composition that is in contact with and/or dried onto a conjugation pad 26 of the test strip. In some embodiments, the detection agent 30 is in a liquid composition (e.g., an aqueous composition) that is contacted to (e.g., added to, deposited onto, and/or the like) the test strip, optionally wherein the liquid composition is combined with a sample prior to, during and/or after contact to the test strip.

A kit of the present invention may comprise an aqueous composition comprising a salt (e.g., NaCl) in an amount of about 1 or 5 mM to about 10, 20, 30, 40, 50, 60, 70, 80, 90, or 100 mM. In some embodiments, the aqueous composition comprises a salt (e.g., NaCl) in an amount of about 10 mM or less (e.g., about 0.5, 1, 2, 3, 4, 5, 6. 7, 8, 9, or 10 mM). In addition or alternatively, the aqueous composition may comprise a buffer and/or buffering agent. Exemplary buffers and/or buffering agents include, but are not limited to HEPES, polysorbate 20 (e.g., TWEEN® 20), Dextran, Bovine serum albumins, proclin, sucrose, polyvinylpyrrolidone (PVP), non-fat milk, Tris buffer, phosphate buffered saline (PBS), and/or a phosphate buffer. In some embodiments, the aqueous composition comprises a buffer and/or buffering agent in an amount of about 0.001%, 0.005%, 0.01%, 0.05%, or 0.1% to about 0.5%, 1%, 1.5%, or 2% v/v of the composition and/or in an amount of about 0.5, 1, 2, 3, 4, 5, 6, 7, 8, 9, or 10 mM to about 11, 12, 13, 14, 15, 16, 17, 18, 19, or 20 mM. In some embodiments, the aqueous composition may include about 0.05% v/v of TWEEN® 20 and about 10 mM of HEPES. The aqueous composition may have a pH of about 6, 6.5, or 7 to about 7.5, or 8. In some embodiments, the aqueous composition may be a buffer and/or may comprise about 10 mM sodium phosphate and about 0.05% TWEEN®-20 and have a pH of about 7.4. In some embodiments, the aqueous composition may comprise the detection agent 30 or the detection agent 30 and/or sample may be combined with (e.g., added to) the aqueous composition.

In some embodiments, a kit of the present invention comprises instructions for using the binding pair and/or test strip.

A method of detecting a target agent 42 (e.g., a pathogen) in a fluid sample 40 may be provided according to embodiments of the present invention. In some embodiments, the method comprises contacting a test strip of the present invention and a fluid sample 40 that may or may not include the target agent 42, and determining whether a signal is present at the test region 14 and/or optionally detecting a signal at the test region 14, wherein the signal at the test region 14 indicates the presence of the target agent 42 in the fluid sample 40. Accordingly, the method may be used to determine and/or detect if the target agent 42 is present in the fluid sample 40. The test strip may be as provided in FIG. 1 or 2. In some embodiments, the method further comprises determining whether a signal is present at the control region 18 and/or optionally detecting a signal at the control region 18, wherein the signal at the control region 18 is a positive control and/or indicates that the detection agent 30 reached the control region 18 and/or that the test strip is working properly. In some embodiments, a method of the present invention and/or determining whether the target agent 42 is present in the fluid sample 40 is carried out without and/or is devoid of a polymerase chain reaction (PCR) step and/or method. In some embodiments, PCR is not needed to determine whether or not the target agent 42 is present in the fluid sample 40. In some embodiments, a PCR step and/or method is performed to confirm whether or not the target agent 42 is present in the fluid sample 40.

In some embodiments, the fluid sample 40 may be a bodily fluid, such as, but not limited to, saliva, a nasal sample, blood, serum, and/or the like. In some embodiments, the fluid sample 40 is an environmental sample and/or food sample (e.g., a food extract). In some embodiments, all or a portion of the fluid sample 40 is obtained from a subject (e.g., a mammal such as a human), the environment (e.g., sewage, water source, etc.), and/or an article (e.g., collected from a food, drink, and/or surface of an article such as a table) and may be in the form of liquid, collected onto an article (e.g., a swap), and/or in the form of a solid. In some embodiments, the fluid sample 40 is as obtained and used (e.g., contacted to a test strip) as provided directly from the source (e.g., subject, environment, and/or article). In some embodiment, the fluid sample 40 is processed and/or prepared prior to contacting the fluid sample 40 to the test strip. For example, upon obtaining a sample from a source, the sample may be combined with a composition (e.g., an aqueous composition optionally comprising a buffer, salt, and/or detection agent 30), dialyzed, purified, concentrated, and/or the like to provide the fluid sample 40. In some embodiments, the sample obtained from a source may go through a process to extract a target agent 42, if present, in the sample such as by combining the sample with a composition (e.g., an aqueous composition optionally comprising a buffer, salt, and/or detection agent 30) that can extract the target agent 42. In some embodiments, the sample obtained from a source may be combined with an aqueous composition optionally comprising a buffer, salt, and/or detection agent 30 and optionally concentrated (e.g., using dialysis and/or ion concentration polarization) to provide the fluid sample 40 that is contacted to a test strip of the present invention. In some embodiments, the fluid sample 40 is directly or indirectly contacted (e.g., added to and/or deposited onto) to the substrate 12 and/or is directly contacted to (e.g., added to and/or deposited onto) to the sample pad 24. In some embodiments, a detection agent 30 and/or a composition (e.g., an aqueous composition) comprising a detection agent 30 is added to a sample to provide the fluid sample 40. Upon contact to the test strip, the fluid sample 40 may move (e.g., by capillary action) through the substrate 12 to the test region 14 and optionally to the control region 18 and/or the adsorption pad 28.

Responsive to binding of the target agent 42 to the GAG 16 and binding of the detection agent 30 to the target agent 42, a signal can be provided at the test region 14, optionally wherein the signal is a first color. Responsive to binding of the detection agent 30 to a control agent 22 (e.g., a control agent 22 at a control region 18), a signal (e.g., a second color) can be provided such as at the control region 18, optionally wherein the signal is a second color. In some embodiments, the method comprises detecting the signal at the test region 14 and/or the signal at the control region 18. Detecting a signal (e.g., at the test region 14 and/or control region 18) may be done using any method known to those of skill in the art. In some embodiments, detecting the signal comprises visually detecting the signal such as visually by eye (e.g., human eye). In some embodiments, detecting the signal comprises electronically detecting the signal such as electronically detecting the signal using a computer, processor, camera, sensor, and/or the like.

A signal (e.g., at the test region 14 and/or control region 18) may be detected in about 30 minutes or less, responsive to contacting a test strip of the present invention and the fluid sample 40. In some embodiments, a first signal is provided at a test region 14 and/or a second signal is provided at a control region 18 of a test strip at about 1, 2, 3, 4, 5, 6, 7, 8, 9, or 10 minute(s) to about 11, 12, 13, 14, 15, 16, 17, 18, 19, 20, 21, 22, 23, 24, 25, 26, 27, 28, 29, or 30 minutes after initial contact of the fluid sample 40 to the substrate 12 and/or sample pad 24 of the test strip.

In some embodiments, a method of detecting a target (e.g., target agent 42) in a fluid sample is provided, the method comprising combining a fluid sample and a binding pair of the present invention and, responsive to the target being present, detecting formation of a complex comprising the first member, the second member and the target, thereby detecting the target in the fluid sample. The fluid sample may be fluid sample 40. For example, the fluid sample may be a bodily fluid, such as, but not limited to, saliva, a nasal sample, blood, serum, and/or the like. In some embodiments, fluid sample 40 comprises a salt (e.g., NaCl) in an amount of about 1 mM to about 10, 20, 30, 40, 50, 60, 70, 80, 90, or 100 mM. In some embodiments, fluid sample 40 comprises a salt (e.g., NaCl) in an amount of about 10 mM or less. In some embodiments, the fluid sample has a pH of about 6 to about 8.

A method of detecting a target in a fluid sample may comprise detecting a signal from a signaling agent and/or from a reporter system, optionally wherein the signal is a first color. In some embodiments, the signal from at least one member of the binding pair that is unbound to the target is different than the signal provided from the at least one member of the binding pair when it is bound to the target and/or in a complex with the target and the other member of the binding pair. Detecting the signal may comprise visually detecting the signal. In some embodiments, detecting formation of the complex comprises detecting the signal from a signaling agent. In some embodiments, the complex and/or the signal may be detected in about 30 minutes or less, responsive to combining the binding pair and the fluid sample, when the target is present in the fluid sample. In some embodiments, detecting may comprise optically, visually, electronically, electrochemically, and/or chemically detecting a signal (e.g., from a signaling agent and/or from a reporter system), a signaling agent, and/or a complex comprising first and second members of a binding pair. Detecting may comprise measuring the color, signal intensity, size (e.g., particle and/or complex size) and/or another unit of measure of a signal or means to detect a complex and/or presence of the target. In some embodiments, surface plasmon resonance is used to detect a signal and/or presence of the target.

In some embodiments, a method of the present invention may be devoid of a PCR step and/or method and/or devoid of an extraction step. For example, a method of the present invention may not need a PCR step and/or extraction step in order to determine if a target is present (e.g., present in a fluid sample). In some embodiments, a sample obtained directly from a source (e.g., from a subject, the environment, and/or an article) is used to combine with a binding pair of the present invention without a step of extracting the target from the sample and/or concentrating the target in the sample. In some embodiments, a sample obtained from a source is combined directly with a binding pair of the present invention and/or contacted to a test strip of the present invention without any intermediary steps to modify the sample.

In some embodiments, a solution comprising a fluid sample and a binding pair of the present invention is provided. A first member of the binding pair may comprise a glycosaminoglycan (GAG) (e.g., a heparin and/or heparan sulfate proteoglycan (HSPG) attached to a first signaling agent, and a second member of the binding pair may comprise a compound (e.g., a protein such as an antibody and/or enzyme) attached to a second signaling agent. A method of the present invention may comprise detecting a pathogen a fluid sample using a solution of the present invention. In some embodiments, at least one member of a binding pair is attached to a nanoparticle (e.g., a gold nanoparticle). In some embodiments, a first member of a binding pair comprises heparin conjugated to a gold nanoparticle. In some embodiments, a second member of a binding pair comprises an ACE2 and/or NTD antibody conjugated to a magnetic nanoparticle. The first member, second member, a complex comprising the first and second members, and/or a pathogen may be optically, visually, electronically, electrochemically, and/or chemically detected according to embodiments of the present invention. For example, referring now to FIG. 38, a first member of a binding pair included GAGs that were conjugated to a gold nanoparticle through a streptavidin-biotin reaction and a second member of the binding pair included an ACE2 or NTD antibody that was conjugated to a magnetic nanoparticle, and the first and second members were provided in a solution with or without the target (SARS-CoV-2 Spike). The solution was incubated with the binding pair for 30 minutes. Then, the solution was magnetically separated for 5 minutes and the presence of the target was visually and optically measured with the results provided in FIG. 38. When the target was present, a clear solution was observed, whereas a red color was observed in the absence of the target.

The foregoing is illustrative of the present invention, and is not to be construed as limiting thereof. The invention is defined by the following claims, with equivalents of the claims to be included therein.

### EXAMPLES

### Example 1

The glycocalyx, a dense “sugary” matrix that coats epithelial tissue cells, is responsible for cell-cell adhesion, extracellular communication, growth factor monitoring, defense against exogenous pathogens, and much more. The major components of the glycocalyx include enzymatic glycoproteins, glycolipids, and proteoglycans. Proteoglycans are heavily glycosylated membrane proteins whose glycan components are mainly glycosaminoglycans (GAGs), or long, linear polysaccharides with repeating units of a uronic acid sugar and an amino sugar.

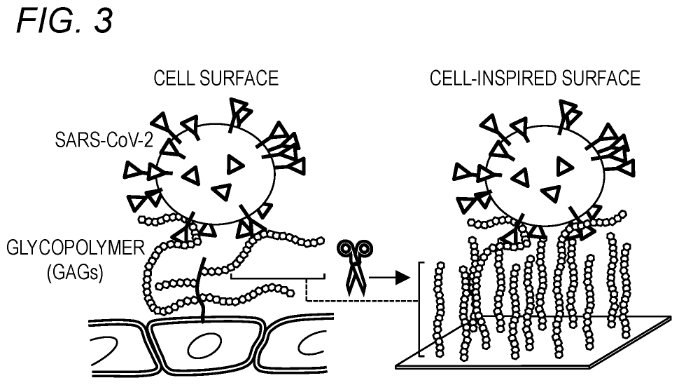

Heparan sulfate (HS) proteoglycans are the most abundant component of epithelial glycocalyx, making up 50-90% of the total sugar composition, followed by chondroitin sulfate (CS). HS is made up of repeating dimeric units of a uronic acid (either glucuronic or iduronic acid) and N-acetylglucosamine, and CS consists of repeating dimeric units of glucuronic acid and N-acetylgalactosamine. Stereochemical composition (i.e., proportions of glucuronic acid versus iduronic acid) and sulfation rates in HS and CS vary greatly depending on tissue types, as well as other physiological conditions such as healthy or diseased tissue status. Viral pathogens often hijack glycocalyx receptor trafficking and signal transduction mechanisms to facilitate entry into host cells (FIG. 3). An important example of this is SARS-CoV-2 (severe acute respiratory coronavirus 2) viral replication cycle and its resultant disease known as COVID-19 (coronavirus disease 2019).

SARS-CoV-2 is a member of the betacoronavirus genus within the Coronaviridae family of viruses; it is a lipid-enveloped, positive-sense, single-stranded RNA virus studded with approximately 30 structural S or “spike” glycoproteins. The spike is a homotrimeric protein composed of many interworking domains. Two of these domains are particularly important to this work: the Receptor Binding Domain (RBD, residues 330 to 530) and the N-terminal Domain (NTD, residues 13 to 296). Furthermore, the spike surface is heavily shielded with 66 N-linked glycans and a varying number of O-glycans.^{[13,14]} SARS-CoV-2 spike's main function is to incite the membrane fusion process by binding to angiotensin converting enzyme 2 (ACE2), situated on the surface of ciliated lung epithelial cells.^{[15-19]} To bind ACE2, the spike must be in an “open” conformation, with at least one RBD in the “up” state.^{[20,21]} In particular, the RBD moves into the “up” state to reveal the Receptor Binding Motif (the RBM), the spike region that makes direct contact with ACE2. Recent works have described the role of the spike's glycans in facilitating and stabilizing the conformational transition from down RBDs to up RBDs^{[15,16,22]} thereby facilitating host-cell invasion. Intriguingly, glycocalyx glycopolymers may also help facilitate SARS-CoV-2 invasion: Esko and coworkers have illustrated that spike binding to HS in the glycocalyx facilitates interaction with ACE2, and incubation with heparin (HEP) induces an increase in spike populations with up versus down RBDs.^{[15,16,30,31,22-29]} Furthermore, other work by Linhardt and coworkers,^{[32]} Fadda and coworkers, and Wade and coworkers^{[34]} have posited HEP binding sites on the spike surface.

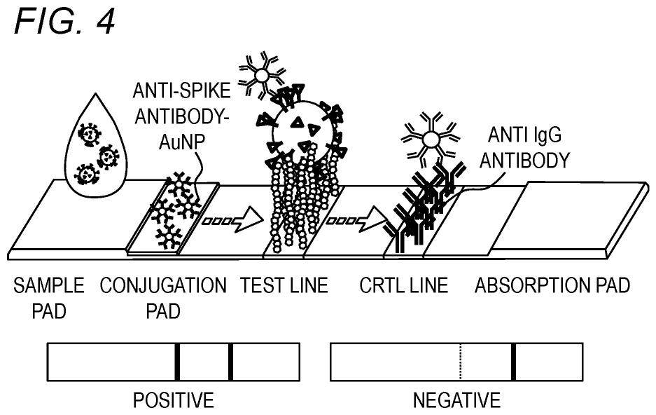

GAGs overall have been underappreciated as potential bioreceptors in biosensors due to their complexity, heterogeneity in sulfation patterns, and low analyte specificity compared to targeted antibodies.^{[11,12,38,39]} Biologically, HS and CS serve as cellular staging grounds: they non-specifically bind many analytes while the specific co-receptors on the cell surface find optimal orientation. Although low binding specificity has been traditionally considered a disadvantageous trait for bioreceptors, without being bound to any particular theory, the discoveries of the present invention suggest that this non-specificity—inspired by glycobiology—coupled with its multivalent binding, can be leveraged to design highly generalizable sensors for the sensitive detection of viruses and viral antigens, and this approach has been applied for sensing SARS-CoV-2 and its variants in a Lateral Flow Strip-based Assay (LFSA) (FIG. 4).

LFSA is an attractive platform for detecting viruses, especially in limited resource settings, due its simplicity, low cost, and rapid signal generation. Typically, “sandwich-type” detection using lateral flow (LF) strips utilize two bioreceptors that bind to the target molecule simultaneously.^{[41-44]} One of the bioreceptors is usually immobilized on the nitrocellulose membrane surface to capture the target analyte, while the other bioreceptor is typically labeled with reporter molecules (e.g., gold nanoparticles, fluorescent dyes, enzymes, etc.) to signal the formation of a sandwich complex in the presence of the analyte. Using traditional bioreceptors, such as antibodies and aptamers as immobilizing agents in LFSA design, requires these receptors to be screened and optimized, as well as necessitating that LF strips be reconfigured, for every desired viral analyte and potential mutant strains. Furthermore, in the case of LFSA sandwich-type detection, pairs of antibodies or aptamers must be screened for both capturing and reporting, which delays sensor design and development.^{[45,46]} In contrast, GAGs could serve as universal capture agents for various viral analytes including mutant strains, resulting in a generalizable LF platform. This could greatly reduce the assay's antibody screening and optimization time, which typically takes anywhere from 12 to 16 weeks.^{[47,48]} This could enable fast adaptation of GAG-based LF strips for current and emerging viruses, and provide cheap and simple to administer viral antigen tests for critically under-tested communities during times of global health crises. In the case of COVID-19, nucleic acid detection methods, such as RT-PCR, are the gold standard in viral testing. However, there is a significant time gap between testing and obtaining results due to the PCR testing capacity limitations. Additionally, areas without accessible RT-PCR testing capabilities are predominantly lower income and/or rural, making this a public health priority as well. Thus, self-administered antigen-based rapid testing would be able to fill the time and resource gaps of the nucleic acid-based testing method for monitoring and containment.^{[49-51]}

Using GAGs as capture probes in LF biosensors introduces two major design challenges: (1) Due to their molecular flexibility, specific binding mechanisms between GAGs and the spike protein are largely unknown and experimentally-determined bound structures remain elusive, and (2) due to their highly heterogeneous composition—i.e., varying stereochemical ratios, sulfation rates, and chain lengths—it is challenging to optimize sensitive and selective LF strips in a robust and reproducible fashion. To address these issues herein, we have integrated rigorous computational methods and extensive experimental system development to create GlycoGrip: a highly sensitive and selective LF strip biosensor for a rapid detection of SARS-CoV-2 spike protein (FIG. 4). Our GlycoGrip LF biosensor is inspired by the glycocalyx. It utilizes GAGs as primary bioreceptors anchored to a test trip to capture the spike protein, while anti-spike monoclonal antibodies labeled with gold nanoparticles (AuAb) are used as reporters. In the presence of the virus, GAGs and AuAb co-bind to the target virus forming the “sandwich” ternary complex and generating color on both the test and control lines in under 30 minutes (FIG. 4, ‘positive’). In the absence of target virus, only color on the control line emerges as AuAb are captured by the control anti-IgG antibodies (FIG. 4, ‘negative’).

Through a recursive feedback loop of experiments and simulations, we have fine-tuned our GlycoGrip sensor and elucidated key mechanisms of how polysaccharides, specifically HS, bind spike during SARS-CoV-2 host cell invasion. Our computational methods uniquely integrated fully flexible, ensemble-based docking procedures considering the entirety of the spike head, with a complete spike glycoprofile, and modeled several GAGs including HS, HEP, CS and dextran sulfate (DEX, a synthetic HEP analog). Our GlycoGrip technology is uniquely tailored to capture and detect SARS-CoV-2 and its rapidly emerging variants and could be applied to detect other pathogenic proteins.

### Establishing Glycocalyx Polymers as Capture Agents for Spike Binding

The concept of taking advantage of GAGs' ability to bind spike in a multivalent manner and employ them as surface-anchored capture agents for detecting SARS-CoV-2 serves as the basis for our work. Esko and coworkers have posited that HS is a necessary co-receptor for SARS-CoV-2 viral infection. They elegantly show that binding of SARS-CoV-2 spike to ACE2 in mammalian cell lines is drastically reduced upon introduction of heparan sulfatase, and the presence of ACE2 alone on a mammalian cell surface is not sufficient for SARS-CoV-2 host-cell invasion, suggesting HS is required. Esko's computational modeling focused on interactions between HS/HEP and the spike RBD, whereas Wade and coworkers^{[34]} as well as Linhardt and coworkers^{[32]} extended the search range for HEP hotspots to include the entire spike head region. Furthermore, Fadda and coworkers^{[33]} posit that a site along the RBD is uniquely suited for binding charged oligosaccharides. Taken together, these works indicate that the structure of SARS-CoV-2 spike might have evolved to be uniquely tailored for binding glycocalyx GAGs.

### Modeling GAG Chemical Diversity

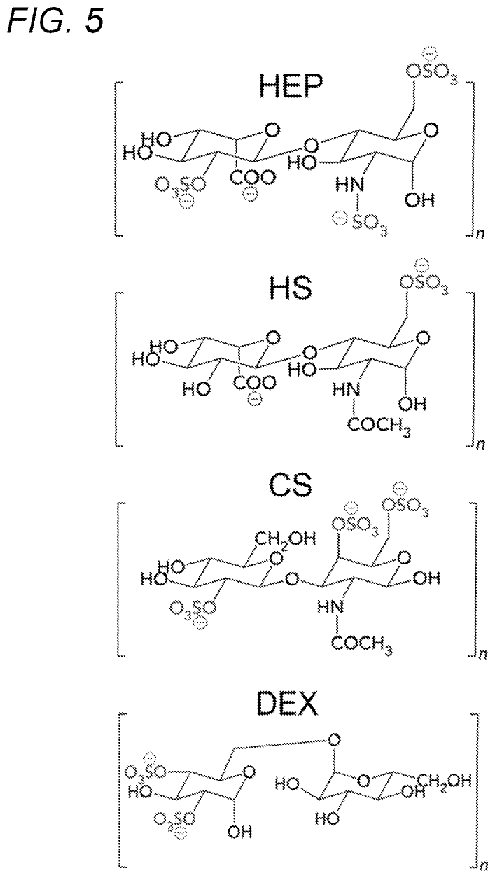

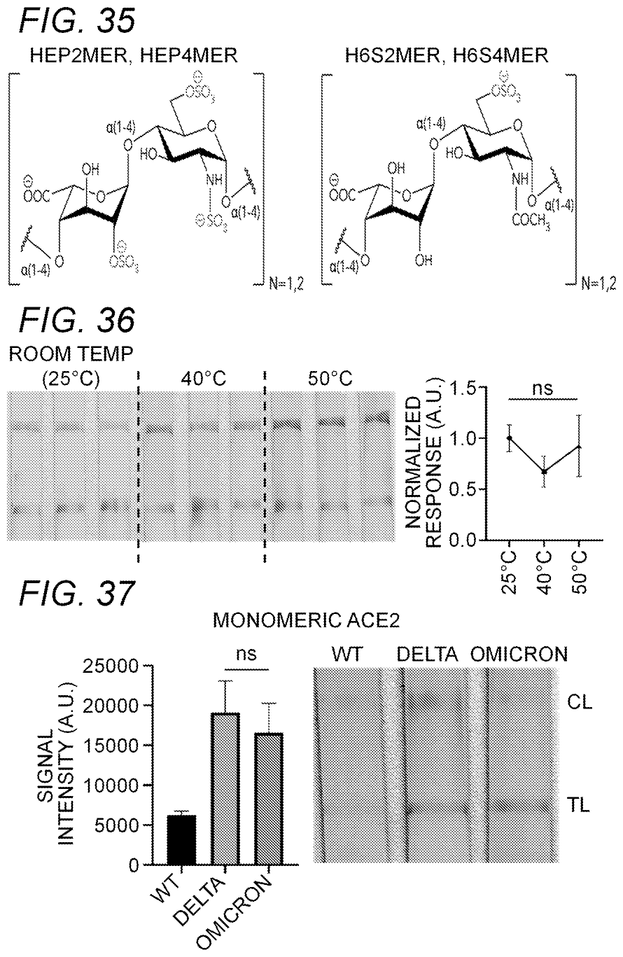

We first sought to identify spike-GAG binding sites to exploit for our sensor. We used an ensemble-based docking protocol that includes docking of multiple GAG identities into various well-sampled/varied protein and glycan conformations, and unbiased searching of the entire spike head. In addition to the most abundant GAGs HS and CS^{[4]}, low molecular weight HEP has been shown to bind effectively to the SARS-CoV-2 spike, as well as induce the RBD down (where the RBM is shielded, thus cannot bind ACE2) to RBD up (RBM exposed, thus ready to bind ACE2) conformation.^{[23,24,26,34]} To capture sufficient GAG diversity, we considered HEP, HS, CS, and DEX to identify which GAG would best capture the SARS-CoV-2 spike on an LF test strip. As docking long polysaccharide chains is intractable—due to combinatorial enumeration of rotational degrees of freedom—we chose to model dimeric (n=1) and tetrameric (n=2) forms of each of our four candidates. Dimeric forms were included to capture highly localized interactions, while tetrameric forms were included to elucidate slightly longer-range effects, i.e., inaccessibility for longer polysaccharide chains. We used MatrixDB^{[52-55]} to build dimeric and tetrameric HEP and HS, and CHARMM-GUI^{[56-59]} to build CS and low sulfated (˜6% sulfated) DEX. FIG. 5 provides ChemDraw images of dimeric forms and FIG. 35 provides additional structures.

### Accounting for Spike Conformations and Surface Accessibility for Binding

To incorporate protein and glycan flexibility in our docking protocol, we docked all GAG models (8 total molecule models) into four different spike conformations extracted from Casalino et al.'s trajectories.^{[4,41]} We used accessibility of the furin-cleavage site as a metric to identify four conformationally unique frames to serve as receptor structures for docking. The polybasic furin-cleavage site (spike residues 674 to 685) is one of the most flexible regions of the spike protein and postulated to bind a myriad of physiological cofactors.^{[29,34,35]} Therefore, we selected four spike conformations based on their degree of accessibility to the furin cleavage site. Furthermore, given each of these GAGs is highly flexible, we thoroughly sampled polysaccharide rotational degrees of freedom by predicting 400 poses per ligand and protein conformation pair. At 8 GAG models, four protein conformations, and 400 predicted poses per pair, this resulted in a total of 12,800 resultant binding modes.

### Selecting Favorable Spike-Gag Binding Sites

To organize our 12,800 predicted binding poses into discernable binding “sites,” we clustered them by their centers of mass. From clustering, we determined 17 distinct spike-GAG binding sites. For easy reference, we have indexed the sites by letter, A through Q. We then derived a “binding site importance score” (Equation 1) to describe the “importance” of each predicted binding site.

Binding Site Importance Score=|(avg binding score of cluster)|*fraction populated (Equation 1)

Out of the 17 unique binding sites, three sites (J, O, and Q) were omitted from further analysis as they are highly buried within the spike and would not be accessible to long-chain GAGs. From the remaining 14 surface binding sites, we identified 7 novel binding sites (F, G, I, K, L, M, N) and validated 7 previously identified binding sites (A, B, C, D, E, H, P). Sites C, B, and D correspond to a “supersite” formed between the Esko^{[24]} and Fadda^{[33]} proposed sites (C, B, and D are analogous sites centered on one of each spike protomers) and sites E and H correspond to a “linking region” posited by Wade and coworkers^{[34]} which connects the Esko and Fadda supersite down to the furin-cleavage site. Our results support the importance of these sites for GAG binding and reaffirms the need to focus on these regions when studying the role of HS in SARS-CoV-2 host-cell invasion mechanism. Novel sites F, L, and M have yet to be described in the literature, but they represent an alternative binding mode for long-chain GAGs on the spike surface. Wade and coworkers connected the Esko and Fadda supersite down to the furin cleavage site along a ridge formed between the left protomer's RBD and the right protomer's NTD. Sites M and L represent sites that could, instead, be used to connect the Esko and Fadda supersite down to the furin cleavage site between a ridge formed by the right protomer's RBD and the left protomer's NTD. Thus, we predict there are two distinct surface paths along which a long GAG could bind to interact with both the Esko and Fadda supersite and the furin cleavage site. This finding highlights the importance of GAGs' multivalent binding mode in attaching to spike surface. For all 14 surface binding sites, we estimated their relative “importance” through our binding site importance score (Equation 1). Sites B, D, E, F, L, and M, all have relatively high importance with scores 0.9, 0.6, 0.6, 0.7, 0.8, 0.9, respectively, indicating a high likelihood for GAG binding at these sites.

### Experimental Characterization of Spike-GAG Binding Affinities by Biolayer Interferometry

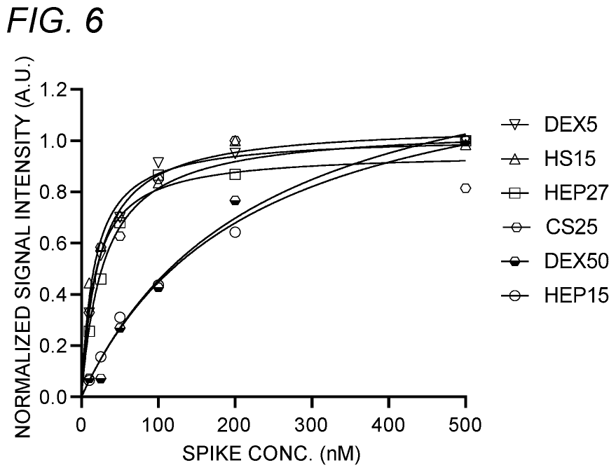

Our ensemble-based docking indicated that all dimer and tetrameric GAGs bound with relatively similar predicted binding energies in each binding site. To better discriminate their affinity to spike, we turned to biolayer interferometry. As the glycocalyx composition is heterogeneous with respect to GAG identity and GAG length, we were interested to evaluate the binding affinity of various GAGs at different chain lengths to the spike: 15 and 27 kDa HEP (HEP15, HEP27), 15 kDa HS (HS15), 25 kDa CS (CS25), and 5 and 50 kDa DEX (DEX5, DEX50). As shown in FIG. 6, all GAGs, at all chain lengths, bound to spike with relatively high affinity. HS15 and CS25 exhibited the highest binding affinities out of all GAGs tested, with K_{D} of 16.7 nM [95% CI; 8 nM-29 nM], 17.9 nM [95% CI; 5 nM-43 nM] respectively. These are followed in order of K_{D} by HEP27, DEX5, HEP15, and DEX50. [60-62] As HS15 shows the highest affinity for spike and considering the ubiquitous presence of HS in the glycocalyx, coexisting around ACE2, it is possible the SARS-CoV-2 spike sequence has undergone selective pressure with respect to binding ACE2 as well as HS. In the context of our sensor construction, although all BLI-tested GAGs showed high affinity for SARS-CoV-2 and could serve as capture agents, we chose to focus on HS and HEP in our current device.

### Constructing a Long-Chain HEP-Spike Model

Although using short (e.g., dimeric and tetrameric) GAG models was necessary to conduct extensive ensemble-based docking protocols, these short GAGs do not fully capture the extent of steric hindrance and torsional constraints that would arise as SARS-CoV-2 spike approaches a long-chain GAG either in an LF test strip or during host cell invasion. Thus, we used our list of highly populated binding sites and literature sites to guide the construction of a more relevant computational model of spike-GAG binding. We used octameric HEP units as a molecular “thread” to “sew” continuously through binding sites B, H, and down to the furin cleavage site, ultimately connecting all these sites with a 40-meric HEP (hep40mer) molecule. Given the symmetrical nature of spike, we repeated this process for all three spike protomers to illustrate the multivalent GAG-binding potential of spike under biological and in vitro assay conditions. In this current work, our model serves to connect our docking results, at the atomic (molecular-) scale, to our experimental results and the biological context, at the macro-scale.

Taken together, our computational models and biolayer interferometry measurements reveal that glycocalyx-derived GAGs are a promising class of molecules to act as capture agents for SARS-CoV-2, as they have high affinity for its spike protein and can bind the spike in multiple binding sites, with various spike conformations, at high valency.

### Mechanistic Insights: GAGs Adapt to Spike Conformation.

Interestingly, the iterative process of computation and experiments for identifying spike-GAG binding has enabled us to unravel mechanistic insights into SARS-CoV-2 host-cell invasion. Accounting for protein and glycan flexibility via ensemble-based docking elucidated two key mechanistic hypotheses: (1) GAGs are likely to bind in multiple compensatory modes to accommodate changes in spike conformation, and (2) the spike's glycans compete with GAGs for certain binding sites on the spike surface yet stabilize other GAG-spike interactions.

### GAGs can Adapt Multiple Binding Modes, Adjusting to Spike Conformations

As mentioned, we predict site B to be a hotspot for GAG binding; according to our binding site importance score it is our #1 ranked site overall, and it is relatively accessible over the course of 1.8 μs. Furthermore, B sits at crucial spot for the spike: “behind” the RBM, and at the interface between the RBD and the neighboring protomer's NTD. Esko posits interactions between low molecular weight HEP and this site could induce transition from a down to up RBD.^{[24]} Fadda elaborates this site has high affinity for negatively charged oligosaccharides.^{[33]} Additionally, Casalino et al.'s simulations illustrate the RBM is one of the most flexible regions in the spike head, second only to the furin cleavage site.^{[15]} Despite drastic differences in protein and glycan topography around the RBM, site B is highly populated in all four protein conformations for all GAG models. Thus, we hypothesize site B may accommodate multiple interconverting GAG binding modes. The differing binding modes at site B may reveal how HEP facilitates the RBD's transition to the up state: by alleviating tight interfacial interactions between the RBD and NTD, and by remaining bound despite drastic RBD conformational changes.

Spike Glycans can Compete with GAGs or Stabilize GAGs at Surface Binding Sites

Some GAG-spike binding sites, most notably site E, are entirely unoccupied by GAGs in specific spike conformations but highly populated in others. Our analysis reveals that spike glycans may compete with, or stabilize GAG binding at the spike surface. In spike conformations 1 and 3, site E is highly populated for all GAGs. When considering data from only spike conformations 1 and 3, site E is ranked #1, above site B, according to our binding site importance score. However, in spike conformations 2 and 4, site E was shown to be completely inaccessible to GAG binding: 0 binding modes for all modeled molecules. Careful inspection of this site reveals that in spike conformations 2 and 4 the glycan at N331_{B} directly occupies site E, whereas in conformations 1 and 3 N331_{B} glycan moves away from site E. Notably, while N331_{B} moves away from site E in conformations 1 and 3, it is still neighboring site E and thus can provide stabilizing hydrogen bonding interactions to GAGs at this site. Thus, glycan N331_{B} serves to shield GAGs from binding at site E, but when site E becomes available, N331_{B}, N122_{C}, and N165_{C} serve to stabilize GAGs bound at this site. This analysis suggests yet another role for glycans in the spike infection process. Casalino et al. have shown that glycans can shield key spike antigenic regions from recognition by potent antibodies, but glycans N165 and N234 can also prop up and stabilize the RBD in the up state. Furthermore, Sztain et al. have shown that N343 facilitates movement to the up state by “pushing” the RBD up through hydrophobic interactions with RBD residues. Here, we show that glycans serves a dual role: they can both shield the spike surface and stabilize GAGs that make it to the spike surface.

### NTD is Ideal for Co-Binding Spike Using GAG-Bound Test Strips

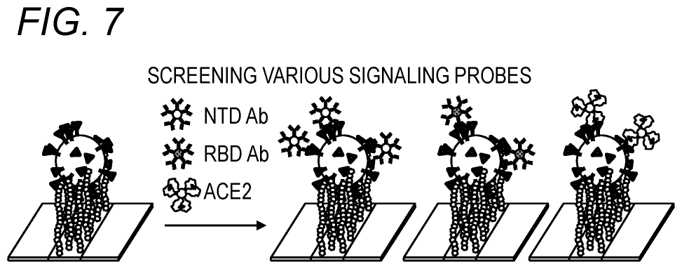

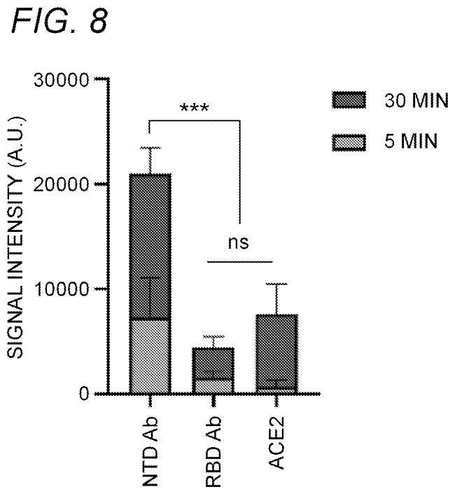

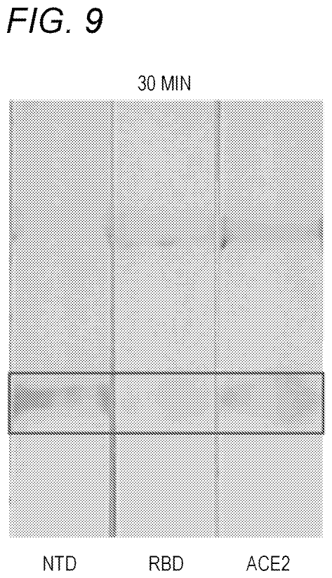

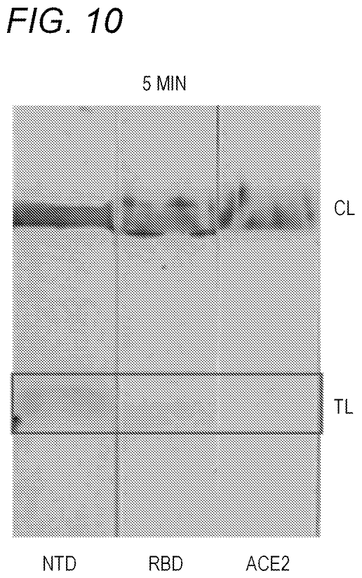

Next, to generate a robust sandwich-based lateral flow assay, we tested a few spike-binding proteins and antibodies for their ability to co-bind spike with surface-bound GAGs. We experimentally screened the following candidates: (1) REGN10933 (RBD Ab), one half of the powerful REGN-COV2 antibody cocktail which binds to a sub-region of the spike RBM, (2) ACE2, the enzyme responsible for binding to spike RBM, which initiates host cell invasion,^{[64,65]} and (3) an NTD binding antibody (NTD Ab) (FIG. 7). As the NTD binding antibody we have used here does not have a specific known epitope, for our computational modeling we have chosen to use the 4A8 NTD binding antibody as a structural stand-in. Initial experimental screening revealed that all candidates form the sandwich-style complex with GAGs when exposed to spike protein for 30 min. To investigate which candidate would generate a positive signal in a shorter detection time, we reduced the incubation time to 5 minutes. Interestingly, using NTD Ab resulted in, by far, the most intense LFSA signal compared to RBD Ab and ACE2 after 5 min, suggesting that the RBD site might be the most ideal for co-binding spike with GAGs (FIGS. 8-10). To explore this result at the atomic scale, we constructed simple spatial models of these complexes: (1) 3×hep40mer with one RBD open spike protomers and bound RBD antibody REGN10933, (2) 3×hep40mer with one RBD open spike protomers and bound ACE2 monomer, and finally (3) 3×hep40mer with all down/closed spike protomers and bound NTD antibody 4A8.^{[66]} From these spatial models, even in the 3×hep40mer-spike complexes, binding of REGN10933, ACE2, or 4A8 could all be theoretically accommodated. Thus, static models alone could not explain why NTD antibodies would provide a much higher LFSA signal relative to RBD-binding biomolecules (REGN10933 and ACE2). None of the predicted binding sites, nor our long-range HEP model, nor the literature proposed sites, overlap with the REGN10933 epitope, the RBM/ACE2 binding domain, or the 4A8 epitope (as illustrated by Figures S14A-B, and 2C, respectively). Therefore, we did not suspect that choice of antibody would have such dramatic impact on the intensity of LFSA signal.

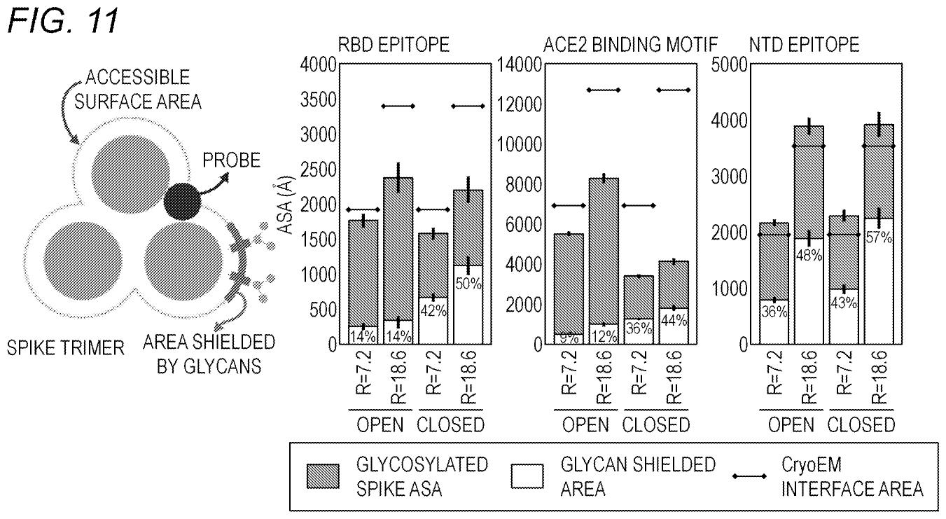

To investigate why an NTD binding antibody might be more suitable than an RBD binding antibody for generating strong LFSA signals in our device, we utilized Casalino et al.'s simulations^{[15]} to interrogate the relative accessibilities of epitopes for REGN10933, ACE2, and 4A8 via accessible surface area (ASA) calculations (FIG. 11). We used the Shrake-Rupley algorithm^{[67]} to calculate ASA for each of these antibody's epitopes (FIG. 11) in both the closed and open spike conformations. We also calculated the size of a “reference” interface from cryo-EM structures, to estimate the required exposed surface area for a successful binding event. To calculate the Cryo-EM reference interface sizes, we used the Protein Data Bank structures for each antibody bound to the spike (PDBID's as follows: 6XDG for REGN10933, 6M17 for ACE2, and 7C2L for 4A8). We then removed the antibody binding partner and calculated surface area of the same interface with the Shrake-Rupley algorithm, as done for Casalino et al.'s simulations.

The resulting ASA plots reveal a high degree of protein self-shielding and glycan shielding exhibited by the two RBD based epitopes (REGN10933 and RBM/ACE2), whereas the NTD provides a consistently exposed epitope for antibody binding (FIG. 11). The reference values for REGN10933 and ACE2 epitopes are out of range of the simulation-visited surface areas for both closed and open spike conformations, with a more pronounced effect in the closed states. In contrast, the NTD epitope reference value is well within the range of simulation-visited surface areas for all probe radii, regardless of closed/open spike conformations. This analysis indicates that the NTD epitope is easily and consistently accessed by AuNP-NTD antibodies, making it a superior choice for LFSA, both for specificity and accessibility. In contrast, many antigenic regions of the RBD are sequestered within the spike trefoil, and only become accessible after the down to up transition is triggered.^{[68-70]} This is likely due to selective pressure on the spike which has driven sequence and structural changes to hide the spike RBD antigenic regions to potentially limit exposure until close-range interactions can occur between the RBD and ACE2.^{[24]} Conversely, NTD structure and accessibility is not affected by spike conformation and thus there are always 3 NTD epitopes available. Even in the best case for an RBD binding antibody, where a spike has begun moving into an open conformation, there may be only 1 or 2 RBDs in the up conformation. Our analysis of Casalino et al.'s simulations provide a reasonable explanation for our observed LFSA results. Taken together, these results indicate that an NTD Ab, of the Abs tested, is an ideal partner for spike recognition with GlycoGrip test strips.

### Sensor Design: Spike and GAG Binding is Driven by Electrostatics and Tuned by Hydrogen Bonding

### Spike Glycans Tune its Surface Electrostatics, Shielding Electrostatically and Sterically

Past works that have commented on the electrostatic potential of the spike surface have ignored electrostatic contributions of spike glycans.^{[24,32]} To better elucidate spike's surface electrostatic profile, we have calculated electrostatic potential maps for wild type (WT) spike with Adaptive Poisson-Boltzmann Solver (APBS) including and excluding glycan contributions. When considering closed spike surface alone (i.e., without including glycans), we indeed see large positively charged regions. These regions tend to contour interfacial regions between protomers. For example, in the closed spike conformation there is a large positive region extending from the top of the spike head, between the RBD and neighboring protomer's NTD, down along this interface between protomers, and then toward the furin cleavage site below the NTD. As expected, these positive regions are postulated by Esko, Linhardt, and Wade as primary sites for long-chain GAG interaction with spike.^{[23,24,34]} Interestingly, when glycans are included in ESP maps, the positive regions remain but they are not as pronounced. Glycans are decorated primarily with electron-rich hydroxyl groups and surround positively charged spike surface regions with significant electron density. Our electrostatic maps underscore the need to include glycans at every step of glycoprotein investigation, as glycans may shield a protein surface sterically or electrostatically. If GAG binding to the spike is dominated by electrostatic interactions, accounting for the electrostatic nature of glycans is important as electron dense glycans may compete with negatively charged GAGs. This pattern of an electron-poor protein surface crowded by electron-dense glycans is also observed in the open spike conformation. Without considering glycans, this putative long-range binding motif-starting at the RBD, running between the RBD and neighboring NTD, down along the protomer interface, and finally to the furin-cleavage site—is evident. In fact, with the exposure of the RBD in the open spike conformation, the positively charged surface on the RBD becomes more evident and extends to the top of the up RBD. But as in the closed structure, upon including glycans, one can see these positive protein surfaces are crowded by electron-rich glycans.

### Solution Ionic Strength Modulates GAGs and Spike Binding

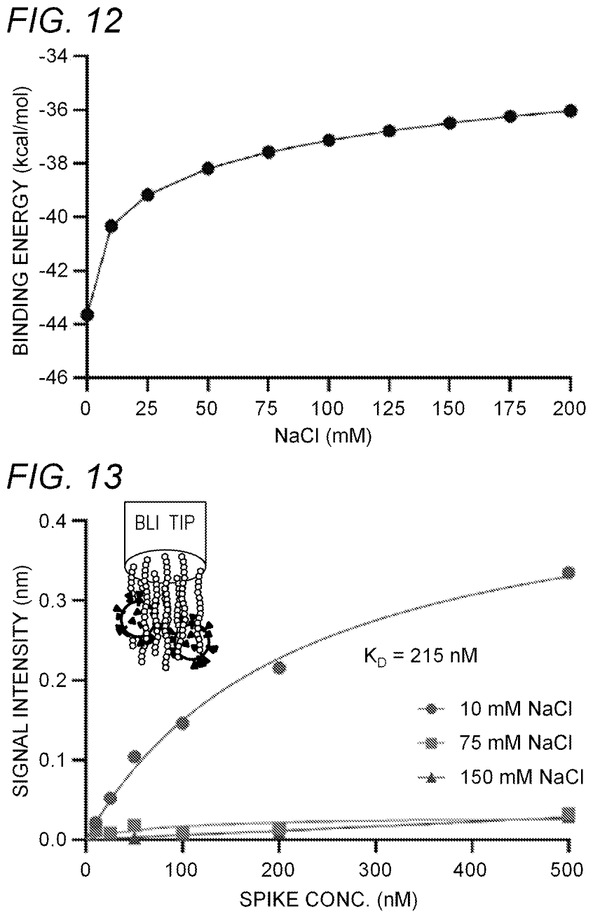

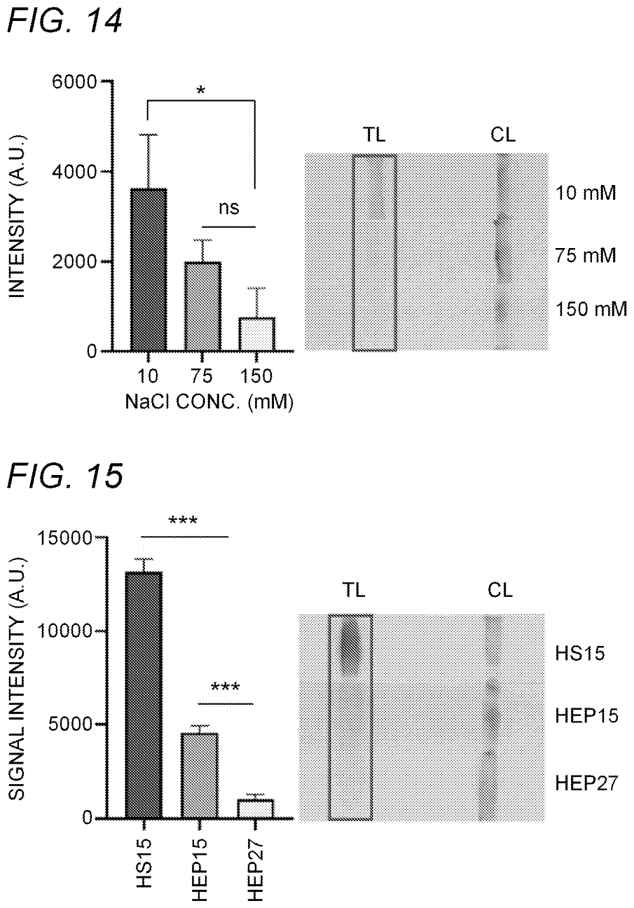

As encounter complex formation and electrostatically driven binding affinities can be modulated by solution ionic strength, we computationally predicted the binding affinity of HEP to spike RBD over a range of ionic concentrations using APBS.^{[71-74]} These results show a clear trend: binding affinity between HEP and spike RBD decreases with increasing solution ionic strength (FIG. 12). BLI results confirm our computational predictions: HEP binding affinity to spike dramatically decreased that K_{D} values could not be determined under the concentration range of spike tested as ionic strength increased (FIG. 13). We have also tested the effect of ionic strength on binding affinity between spike and the NTD antibody. The measured binding affinity between NTD Ab and spike slightly decreased (changed from 52 nM to 83 nM) as NaCl concentration decreased, suggesting that the Ab binding affinity is much less dominated by electrostatics. Next, we monitored the intensity of LFSA signals under three NaCl concentrations (10 mM, 75 mM, 150 mM). As shown in FIG. 14, 10 mM NaCl solution conditions resulted in a 4.7 times more intense signal compared to 150 mM solution conditions (p<0.05). Thus, lower ionic strength yields a higher signal intensity in our LFSA device.

Electrostatics Isn't Everything: HS Binds with Higher Affinity to Spike than HEP.

As mentioned, the prevailing hypothesis in HEP/HS interaction with SARS-CoV-2 spike is that binding is electrostatically driven. [23-26,31,32,34,75,76] One intriguing result of ours complicates this, otherwise, straightforward spike-GAG electrostatic binding model. Our BLI measurements show that HS15 binds to spike with a greater affinity (16.7 nM K_{D}) than the highly sulfated/highly charged HEP15 of the same molecular weight: (215 nM K_{D}). Due to the varied nature of the spike electrostatic surface—i.e., large, positively charged patches obscured by electron dense or electroneutral glycans—this likely indicates that, while electrostatics is a major initial driving force of spike-GAG binding, it is not the only driving force. In fact, at close range, hydrogen bonding interactions and appropriate moderation of electrostatics likely allows HS to bind with higher affinity to spike than highly charged HEP. Additionally, due to the highly charged nature of HEP, the spike-HEP interaction may incur a higher desolvation penalty than the less negatively charged HS.

### HS and HEP are Optimal Glycocalyx-Inspired LFSA Capture Agents

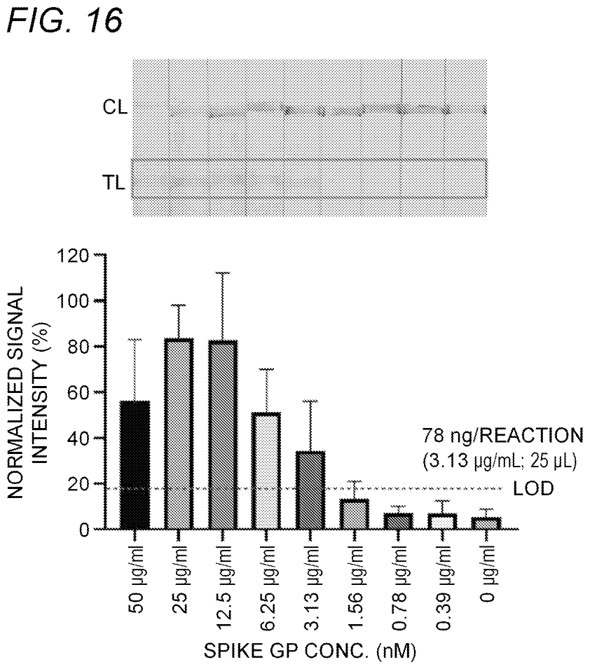

Applying our conditions elucidated by computational and experimental investigations thus far—i.e., use of NTD conjugated AuNP and low ionic strength conditions—we compared test strips employing HS15, HEP15, and HEP27 as capture probes (FIG. 15). HS15 showed the highest signal intensity, in agreement with our BLI results illustrating HS binds spike with highest affinity (lowest K_{D}). Interestingly, longer chain length HEP (HEP27) resulted in lower signal intensity. While not wishing to be bound to any particular theory, we hypothesize that this may be due to the negatively charged nature of the nitrocellulose membrane repelling long-chain, negatively charged GAGs during strip preparation. Since both HS and HEP showed robust and rigorous LFSA bands, we tested the analytical performance of both. Presence of spike protein was detectable as low as 78 ng/reaction (3.13 μg/ml, 25 μl), and the detectable range was 78 ng/reaction-1250 ng/reaction (3.13 μg/ml-50 μg/ml) with the naked eye for both HEP (FIG. 16) and HS based LFSA. The limit of the detection (LOD) for HS and HEP were similar, despite our BLI indications that HS may be a better spike binder, suggesting that the LOD is most likely dictated by the NTD binding affinity. HEP LFSA signals provide a larger range of observably intense signals to the naked eye, and it is more cost effective than HS, which is certainly important when looking to mass produce testing kits for viral outbreaks. Therefore, we continued to design our GlycoGrip biosensor with HEP as the surface-anchored GAG.

### GlycoGrip is a Rapid, Sensitive, Stable, and Selective Assay for the Detection of SARS-CoV-2

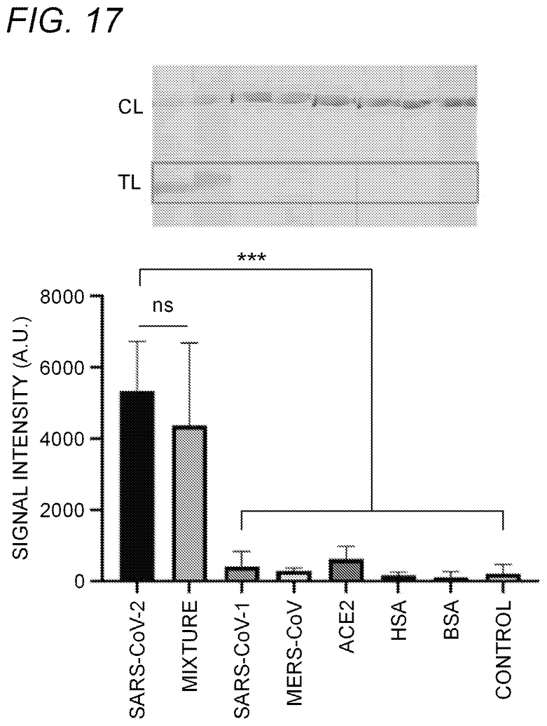

We tested the selectivity of our GlycoGrip biosensor against related beta-coronavirus spike glycoproteins (SARS-CoV spike and MERS-CoV spike S1 domain), as well as biologically relevant proteins likely to be found in patient samples (ACE2, Bovine Serum Albumin, BSA, and Human Serum Albumin, HSA). As shown in FIG. 17, positive bands were observed only when GlycoGrip LF strips were treated with SARS-CoV-2 spike, whereas treatment with other beta-coronavirus spike proteins and biologically relevant “distractors” did not indicate positive test results. Moreover, when tested with a mixture of SARS-CoV-2, SARS-CoV, MERS-CoV spike glycoproteins, band intensity was similar to the pure SARS-CoV-2 spike band. These results suggest that our GlycoGrip LF biosensor can selectively detect the SARS-CoV-2 in more complex solutions, unencumbered by the presence of other beta-coronavirus spikes or by other proteins commonly found in patient samples. This minimizes the possibility of undesirable false positive test results.

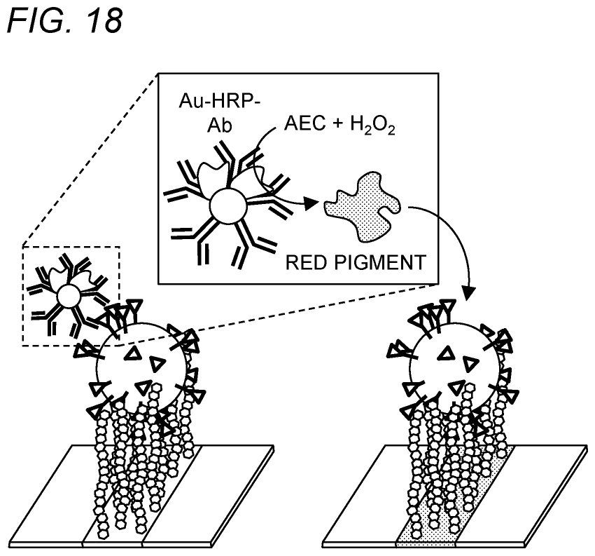

To further enhance the sensitivity of our GlycoGrip LF biosensor, we incorporated a reporter system (horseradish peroxidase (HRP) and 3-amino-9-ethylcarbazole (AEC)) (FIG. 18). NTD Ab tethered with multiple HRP enzymes were used as a signaling probe to enhance the sensitivity by catalytic reaction. Through the enzymatic reaction, water-insoluble red colored chromogenic products were released on the test line which enhances the signal intensity. The LOD with this enzymatic signal enhancement mechanism was estimated to be 19.5 ng/reaction (0.78 μg/ml, 25 μl), enhanced 4-fold compared with unamplified results.

### GlycoGrip Detects Spike in Human Saliva Samples

GlycoGrip can serve as a rapid test whereby samples can be self-collected from one's own saliva in a simple, non-invasive fashion without the need for specialized equipment or personnel. The benefits of this are two-fold: (1) LFSA tests have the potential to reach a wider testing population, and (2) removing the specialized personnel requirement reduces extra cost and eliminates direct contact between infected and non-infected persons. Moreover, collecting saliva samples as opposed to nasal samples has a higher likelihood of indicating positively for both symptomatic and asymptomatic SARS-CoV-2 carriers, as nasal collection has shown high variability in sample integrity due to sampling procedure differences on an individual basis.^{[78,79]} To test GlycoGrip performance in human saliva samples, we introduced a range of spike concentrations into human saliva samples. Sensing proteins in complex fluid such as saliva can be challenging as it contains many other biomolecules that could limit or compete with spike-GAG binding interactions. In the context of sensing SARS-CoV-2, it has been recently reported that glycoproteins in saliva, such as mucin proteins (MUC7, MUC5B) and neutrophil defensins, may bind to and interact with the spike.^{[80]} Thus, these complex glycoproteins may then compete with HEP or Ab for binding sites on the spike surface.^{[80]} Remarkably, the LOD of our GlycoGrip in saliva samples was 39 ng/reaction (1.56 μg/ml, 25 μl) comparable to buffer conditions (LOD: 19.5 ng/reaction) (FIG. 19 for summary of GlycoGrip LF results). These results indicate the strong feasibility of applying GlycoGrip technology to clinical samples.

In addition to sensitivity and selectivity, sensor stability is a vital factor for sensor distribution and storage. To test GlycoGrip's stability, we stored same-day-fabricated GlycoGrip sensor strips in a plastic bag with desiccant at room temperature and tested signal intensity after varying lengths of storage (0, 11, and 47 days). We saw no significant decrease in the signal intensity over 47 days, which indicates that our GlycoGrip LF biosensor is stable for at least 47 days, and most likely much longer.

### GlycoGrip Detects the Presence of SARS-CoV-2 Spike Variants

New strains of SARS-CoV-2 began emerging as early as summer of 2020 and, at the time of this publication, the World Health Organization has highlighted four variants of concern (Alpha (B.1.1.7), Beta (B.1.351), Gamma (P.1), and Delta (B.1.617.2)) and four variants of interest (Eta (B.1.525), Iota (B.1.526), Kappa (B.1.617.1), and Lambda (C.37)).^{[81,82]} These variants exhibit key mutations in the spike protein (as well as mutations in other SARS-CoV-2 structural and non-structural proteins) that are postulated to directly translate to increased infectivity and/or increased immune-system evasion ability. To ensure our GlycoGrip technology would signal positively for patients infected with these emerging SARS-CoV-2 strains, we have tested variant full spikes with our LFSA technology.

Characteristic Mutations of Alpha, Beta, Delta Spike Variants do not Interfere with GAG Binding Sites

We hoped to assess at the molecular scale whether variations in spike sequence and structure, as seen in emerging SARS-CoV-2 variants, could impact binding to HEP on the LFSA test strip. While, at the time of this publication, there are several cryo-EM structures of Alpha, Beta, and Delta SARS-CoV-2 spikes in the Protein Data Bank, [83,84] unfortunately, each of these structures contain unresolved regions corresponding to highly flexible loops. Thus, we constructed computational models of the Alpha, Beta, and Delta spike variants from our refined closed WT spike structure. We then aligned our trivalent spike-hep40mer complex to these variant structures and visually inspected for overlap between mutation points with proposed hep40mer binding regions. From these structures, we observed no overlap or clash between HEP and mutations characterizing Alpha and Beta spike variants. This suggests that GAGs will still likely capture Alpha and Beta spikes under LFSA conditions, as we have proposed in the WT case. In the case of the Delta spike, interestingly, we observed there are several mutations (L452R, T478K, and P681R) that increase the number of positively charged residues along the posited HEP binding cleft. Furthermore, WT spike protein and its glycans constitute an overall absolute charge of −32, while the overall charge of the Delta spike protein, with the same glycoprofile, is −17. Thus, the Delta spike exhibits a drastic increase in total charge as a result of changes in sequence.

As discussed, past work has highlighted the importance of large positively charged regions on the spike surface. [23,24,34] To ensure mutation points in spike variants do not disrupt these positive patches, we have also calculated electrostatic potential maps for Alpha, Beta, and Delta spikes. While there are some differences in surface electrostatic potential compared to WT, noticeably in the trefoil region between neighboring RBDs, our postulated hep40mer binding region remains largely positively charged at the protein surface for Alpha, Beta, and Delta spike variants. This again supports that binding of HEP to spike could be electrostatically driven, and these electrostatic interactions are not affected by the key mutations in new spike variants.^{[85]} One could further hypothesize that an evolutionary advantage exists to maintain the spike's ability to bind glycocalyx polymers. This would be supported by the fact that in Alpha and Beta spike variants there exist no single point mutations along the putative HEP binding cleft. Additionally, the Delta spike variant exhibits three mutations to positive residues along the putative HEP binding cleft (L452R, T478K, P681R) which would further potentiate the ability of spike to bind to negatively charged GAGs. Finally, although we have predicted a long-chain model for HEP binding on the spike surface based on docking studies with the WT, we note that our results indicate that there are at least 14 sites on the WT spike surface where GAGs can bind. Thus, if new spike variants emerge with mutations that interfere with our proposed long-chain HEP binding site, there is still potential for GAGs binding to spike via other long-chain modes according to our proposed multi-site binding model.

### Biolayer Interferometry Confirms Alpha, Beta, Delta Spike Variants Bind to HEP

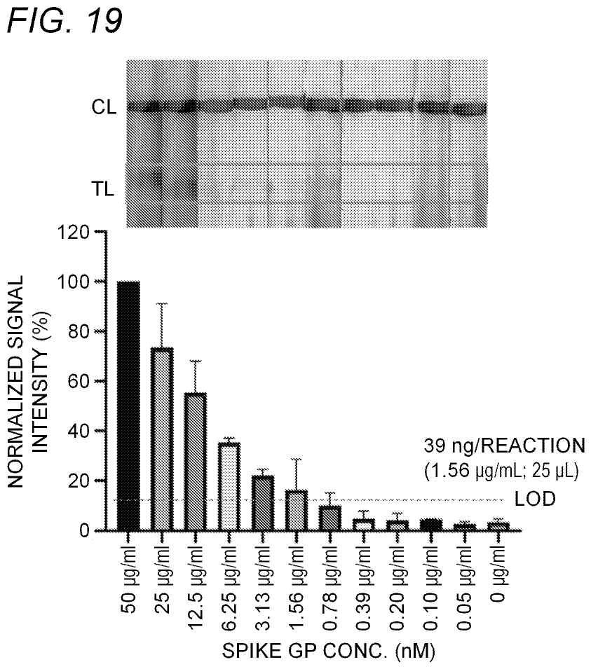

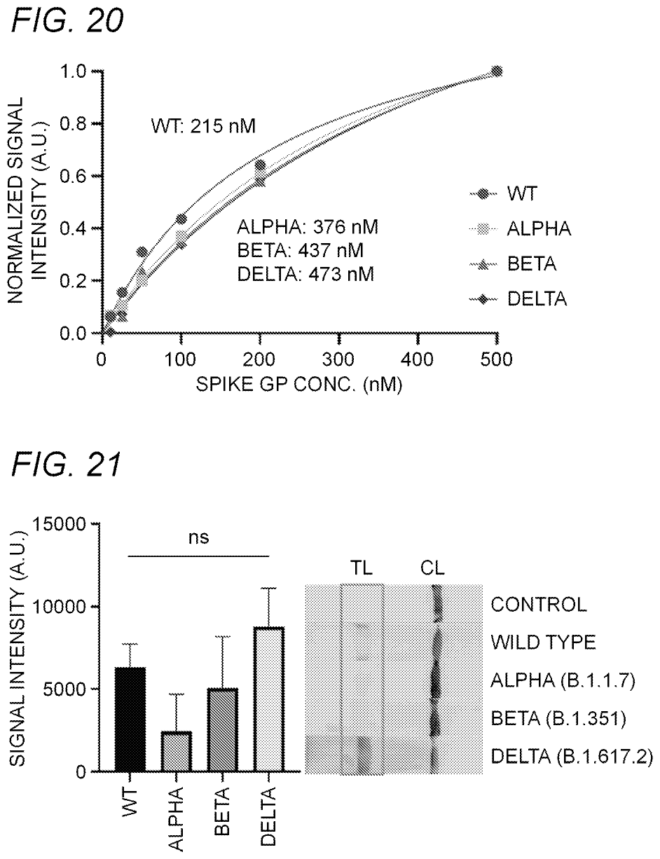

To confirm this in silico prediction, i.e., that GlycoGrip can bind and signal positively for emerging SARS-CoV-2 spike variants, we measured binding affinity of HEP15 to full-length trimeric Alpha, Beta, and Delta spikes with BLI (FIG. 20). Alpha, Beta, and Delta spikes all bound to HEP15 with comparable binding affinity to WT spike, despite their characteristic point mutations and deletions. This result supports both our posited hep40mer binding mode and our electrostatic potential maps: HEP binding is not perturbed by mutations exhibited in the SARS-CoV-2 Alpha, Beta, and Delta strains. This result underscores the power of using GAGs as the capture probe for SARS-CoV-2 spike sensing. To complete the profile of our sandwich-style LFSA detector, we measured the binding affinity between NTD Ab and each spike variant. Binding affinities of variant spikes to the NTD Ab were decreased compared to the WT spike, however, this is to be expected as each variant exhibits characteristic mutations in the NTD. From all variants, the alpha variant showed the lowest binding affinity, most likely due to two key deletions in the NTD which are characteristic of the Alpha variant, one of which is within the N2 loop and one within the N3 loop, with both loops being key for antibody recognition. However, these results reflect another key feature of GlycoGrip: modularization. Since the choice of capture probe and signaling antibody are decoupled in GlycoGrip's design, selecting a new signaling antibody largely does not impact the performance of GAGs to capture analytes.

### Alpha, Beta, Delta Spike Variant Detection on GlycoGrip Strips

Encouraged by our BLI results, we then tested Alpha, Beta, and Delta spike on the GlycoGrip LF test strip. As shown in FIG. 21, all variants exhibited positive bands on the LF test line, and the currently circulating highly infectious Delta variant, can be detected, demonstrating the universality of using glycopolymers as viral capture agents. Our GlycoGrip LF could be rapidly adaptable to newly emerging SARS-CoV-2 variants which is an important aspect of point-of-care sensing platforms for viral pathogen detection.

We have harnessed the power of the glycocalyx and its glycosaminoglycans to serve as capturing agents within an LFSA “sandwich” binding assay to develop our GlycoGrip sensor. Our rigorously applied lock-step integration of experiments and computational modeling allowed us to achieve desired conditions for GlycoGrip and provide mechanistic insights into GAG and spike binding interactions. We have demonstrated the first use of GlycoGrip for detecting wild type SARS-CoV-2 spike as well as the newly emerged SARS-CoV-2 spike variants, Alpha, Beta, and Delta. We have shown that SARS-CoV-2 spikes can be detected on GlycoGrip LF strips and, due to specificity of the chosen NTD-based signaling antibody, we saw no cross-reactivity to SARS-CoV, MERS-CoV, HSA, or ACE2 in buffer or in human saliva. We have also seen that modifying solution ionic strength and GAG length can enhance LFSA signals, along with traditional signal enhancement systems.

In addition to sensor design, we used our extensive ensemble-based docking results to provide biologically relevant mechanistic insights into SARS-CoV-2 host cell invasion mediated by spike-GAG binding. We have confirmed literature proposed sites as well as provided identified seven novel GAG binding sites on the spike surface. Collectively, a clear picture emerges: GAGs in the glycocalyx bind nonspecifically, yet tightly to spike, with potential for multiple long-chain GAGs binding spike at the same time. Additionally, our work highlights, once more, the importance of modeling glycans when studying spike dynamics and interactions. While not wishing to be bound to any particular theory, we predict spike glycans may play a role in shielding the spike surface from incoming GAGs, but once GAGs reach the surface, glycans are likely to support GAGs via hydrogen bonding interactions.

The power of GlycoGrip lies in its modularity and generalizability. Many pathogens—including viruses, bacteria, and parasites—exploit the glycocalyx for cell adhesion. Thus, these pathogens, and/or their characteristic antigens, may be captured by GAGs on a GlycoGrip strip. The need for one antibody instead of two (as required for constructing traditional sandwich-type LFSA sensors) will significantly shorten the screening time [45,46] when applied towards a new pathogen or variant, as well as lower the cost (ca. 100 times) as compared to current LFSA technologies.

GlycoGrip can be used as a widespread tool for capturing and detecting current and emerging viruses. GlycoGrip can be extended for rapid screening and detection of future pathogenic infections. Loss of life prevention in public health crises requires quick detection and disease containment. We specifically note that traditionally medically underserved, and therefore undertested, populations have the hardest time identifying communal outbreaks because they lack access to RT-PCR, a technique that requires highly skilled laboratory staff and expensive instrumentation. In the case of SARS-CoV-2 spike, we have shown that GlycoGrip can detect rapidly emerging variants. This not only speaks to the generalizability of GlycoGrip, but also to its robust longevity over the course of a real-time sustained global health crisis. One could envision GlycoGrip as a synthetic glycocalyx able to trap pathogenic antigens, and coupled with antibodies, used to test patients within minutes.

### Materials & Methods

### Computational Methods

### SARS-CoV-2 Spike and Sulfated Polysaccharide Docking.

In this work we considered heparin (HEP, in this work specifically IdoA2S-GlcNS6S), heparan sulfate (HS, in this work specifically IdoA-Glc6S), chondroitin sulfate (CS, in this work specifically GlcB2S-GlcN4S6S), dextran sulfate, (DEX, in this work specifically GlcA-GlcA2S4S) as potential binding partners for SARS-CoV-2 spike. As docking of long chain polysaccharides to large protein structures is combinatorically intractable, we modeled small dimer and tetramer structures of each sulfated polysaccharide. Dimeric sulfated polysaccharides were modeled with the intention of capturing highly localized interactions, while tetrameric sulfated polysaccharides were modeled with the intention of capturing steric hindrance effects encountered with larger substrates. HEP and HEP6S dimeric and tetrameric structures were constructed with MatrixDB, [52-55] CON and DEX dimeric and tetrameric structures were built with CHARMM-GUI Glycan Builder. [56-59]

To predict potential locations of sulfated polysaccharide binding to spike, we conducted extensive unbiased docking with AutoDock Vina. [87,88] Using exposure of the furin cleavage site as a metric to detect conformational changes, we selected four spike coordinates from Casalino et al. closed spike trajectories (amarolab.ucsd.ed/covid19.php.) These spike structures were prepared as described by Casalino et al.

Two structures must be well defined in any docking protocol: the receptor/macromolecule to be docked into and the ligands/small molecule to be docked. To avoid bias of docking results to only certain regions of spike, for each of the four spike structures, we defined the “receptor” to be any location on/within the spike head (residues 13 to 1140). To define these receptors in AutoDock Vina, we generated grids centered on the spike head, with large enough dimensions to encompass the entire head, and with default grid spacings. For complete listing of grid coordinates and grid values see SI Methods. Since we have used four distinct spike conformations as receptors in these docking procedures, we thus have incorporated a degree of protein flexibility. To characterize the structural diversity of each of these protein structures we have calculated the root mean square deviation (RMSD) between chains for each spike sub-domain of interest.

To identify as many binding sites as possible, we conducted 20 replicates of each docking procedure and requested AutoDock return 20 predicted binding modes per docking replicate—where one docking procedure would entail docking one dimer or tetramer sulfated polysaccharide to each spike conformation. As a result, we predicted 400 binding modes for each dimeric or tetrameric sulfated polysaccharide in each spike conformation, resulting in 12,800 predicted binding modes total: 400 binding modes per molecule, by 8 molecules (dimeric and tetrameric versions of each sulfated polysaccharide), by 4 protein conformations, is 12,800 total. To parse all 12,800 predicted binding modes into discernable binding “sites”, we clustered all these resultant poses by their centers of mass using k-means clustering through python's scikitlearn. A knee/elbow locator algorithm was used to identify the optimal number of clusters.^{[89]} We then derived a binding site “importance” metric to rank binding sites according to average binding score and relative population in that site. The top “important” binding sites were then inspected by eye through Visual Molecular Dynamics (VMD)^{[90]} to determine important binding factors governing each of these sites.

SARS-CoV-2 Spike and hep40mer Model System.

The fully glycosylated SARS-CoV-2 spike model used in our hep40mer modeling is based on an experimental cryo-EM structure of the spike in the closed state where all RBDs are in the down conformation (PDB: 6VXX).^{[91]} To improve the accuracy of our model, fully resolved RBD and NTD loops were incorporated from another closed spike structure (PDB: 7JJI).^{[92]} We note that utilization of the 7JJI structure in its entirety was not ideal as this structure is known to be more compact than 6VXX due to the presence of a fatty acid ligand resolved in the RBD.^{[92]} The complete glycoprofile was replicated from Casalino et. al. Protonation state assignment was performed for spike glycoprotein with complete glycans modeled with stand-alone PropKa (so that glycan atoms could be considered during calculation),^{[93,94]} histidine protonation states were assigned via PropKa through Schrodinger's Protein Preparation Wizard.^{[93-95]} Protonation states of all titratable residues were then compared to those assigned in Casalino et al.^{[15]} for consistency. To propose a long range, HEP binding site along the spike surface, we considered both literature-proposed binding sites, as well as proposed binding sites from our own docking simulations. We considered only surface sites, and the most highly ranked sites were prioritized for inclusion in long range binding mode construction. To generate relaxed HEP conformations for building hep40mer, we conducted 6 replicates of 50 ns of NVT equilibrium molecular dynamics simulations of hep8mer in a water box with NAMD.^{[96,97]} From the resultant 300 ns of HEP simulation, we clustered those frames according to conformation. 95% of all hep8mer coordinates from these simulations could be clustered according to six conformational clusters. Hep8mer coordinates representing the frame closest to each cluster center were used as hep8mer units to fill necessary coordinates between docked poses predicted from AutoDock Vina. Molefacture, a VMD based modeling tool, was used to ensure there were no clashes between protein atoms and ligand atoms.

### Accessible Surface Area (ASA) Analysis.

ASA analysis was done using the measure sasa command built-in to VMD^{[90]} along with extra protocol established by Casalino et. al. The ASA analyses were performed by considering the antigenic regions in the NTD (residues 143-153, 245-259) and the RBD (residues 403-406, 416-422, 453-456, 473-478, 484-498). Additionally, ASA analysis was performed on the canonical RBM/ACE2 binding site (residues 437-508). Calculated ASAs are shown for two probe radii: 7.2 Å and 18.6 Å.^{[98]} The reference interface areas were calculated from cryo-EM structures as follows: REGN10933 antibody bound to spike-RBD (PDB: 6XDG^{[63]}), 4A8 NTD antibody bound to spike-RBD (PDB: 7C2L^{[66]}), ACE2 bound to spike-RBD (PDB: 6M17^{[64]}).

### System Construction for Ionic Concentration Effect Monitoring:

To investigate the effect of ionic concentration on HEP binding affinity, a HEP octamer (hep8mer) was docked to the RBD of an RBD/ACE2 complex (PDB: 6M17) using Schrodinger's Induced Fit Docking protocol.^{[99-101]} This cryo-EM structure was prepared by removing the B⁰AT1 dimer chaperone coordinates manually with VMD,^{[90]} and N-glycans were added on the ACE2 and RBD as done in Barros et al.^{[20]} The ACE2/RBD/hep8mer construct was inserted into a lipid bilayer patch of 225 Å×225 Å with a composition similar to that of mammalian cell membranes (56% POPC, 20% CHL, 11% POPI, 9% POPE, and 4% PSM).^{[102]} The resulting system was then embedded into an orthorhombic box of explicit TIP3P waters.^{[103]} The system was ionized with Na+/Cl− ions at 150 mM for all simulations, unless otherwise specified. All-atom MD simulations were performed on the Frontera supercomputer at the Texas Advanced Supercomputing Center (TACC) using NAMD 2.14^{[96,97]} and CHARMM36m all-atom additive force fields.^{[58,104,105]} Minimization and equilibration were performed in four steps. In the first step, while keeping all the coordinates fixed but the lipid tails, the system was subjected to an initial minimization of 10,000 steps using the conjugate gradient energy approach, followed by an NVT equilibration of 0.5 ns at 1 fs/step, where the temperature was gradually increased from 10 to 310 K. In the second step, positional constraints on lipids head, water and ions were lifted, and the system was NPT-equilibrated for 0.5 ns at 1.01325 bar and 310K with the protein, glycans, and hep8mer harmonically restrained using a spring constant of 1 kcal/mol/Å². Then, the restraints on protein and glycan atoms were removed and the equilibration was extended by 10 ns. Next, restraints on hep8mer atoms were removed to allow the entire system to equilibrate for an additional 10 ns. Finally, MD simulation production runs were performed and 3 replicas of ˜500 ns each were collected.

APBS was used to estimate binding affinity between hep8mer and spike RBD at varying ionic concentrations. Binding affinity was calculated according to an appropriate thermodynamic cycle by calculating binding energy in a homogeneous reference medium (dielectric constant=4), and then by calculating the solvation free energy difference between the homogenous reference state and non-homogenous target state (dielectric constant=78). (apbs.readthedocs.io/en/latest/using/examples/binding-energies.html). Binding energies were calculated for RBD-hep8mer complexes in the following NaCl concentrations: 0.0M, 0.01M, 0.025M, 0.05 M, 0.075M, 0.10M, 0.125M, 0.150M, 0.175M, 0.200M.

### Experimental Methods

### Materials

Human Serum Albumin (A3782), Sucrose (S0389), AEC staining kit (AEC101), biotin-heparin (B9806), heparan sulfate (H7640) were purchased from Sigma-Aldrich. Biotin-PEG3-amine (BG-17) was purchased from G-Biosciences. Tween 20 (J20605-AP) was purchased from thermo scientific. Sodium chloride (BDH9286) was purchased from VWR. Bovine serum albumin (105033) was purchased from MP biomedicals. Gold nanoparticles (15703-20) were purchased from Ted Pella Inc. N-terminal domain binding antibody (LT-2000) and HRP modified N-terminal domain binding antibody (LT2010) were purchased from Leinco Technologies. Receptor domain binding (RBD) antibody (Clone REGEN10933; CPC511B) was purchased from cell sciences. Rabbit Anti-Human IgG (ab6715) and Goat Anti-Mouse IgG (ab6708) were purchased from abcam. Biotin-chondroitin sulfate (25 kDa) (CS-Biotin-25k), biotin-dextran sulfate (50 kDa) (DES-Biotin-50k), and biotin-dextran sulfate (5 kDa) (DES-Biotin-5k) were purchased from HAWORKS. Biotin-heparin (27 kDa) (HP-207) was purchased from Creative PEGWorks. Human saliva pooled from human donors (991-05-P) were purchased from LEE Biosolutions. Nitrocellulose membrane (FF120HP), sample pad (Whatman CF4 dipstick pad), and absorbent pad (Whatman standard 17) were purchased from Cytiva. SARS-CoV-2 Spike protein (40589-V08H4), SARS-CoV S1 (40150-V05H1), MERS-CoV S1 (40069-V08H) were purchased from Sino biological. SARS-CoV-2 Alpha (B.1.1.7) spike (10796-CV-100), SARS-CoV-2 Beta (B.1.351) spike (10786-CV-100), and SARS-CoV-2 Delta (B.1.617.2) spike (10878-CV-100) were purchased from R&D systems. Fc tagged human ACE2 (AC2-H5257) were purchased from Acro Biosystems. Streptavidin modified BLI biosensor tips (18-5019), and anti-human IgG Fc Capture (AHC) BLI biosensor tips (18-5060) were purchased from Sartorious.

### Biolayer Interferometry

To measure the binding affinities of polysaccharides, biolayer interferometry (BLI) was used. Polysaccharide modified tips were prepared by the streptavidin-biotin methods. Streptavidin tips were functionalized with 1 mg/ml of biotin-polysaccharides (40 μl) in a kinetic buffer (10 mM HEPES, 10 mM NaCl, Tween 20 0.05%, pH 7.4) for 180 sec. Polysaccharides modified tips were incubated with various concentrations of spike proteins from 0 to 500 nM in a kinetic buffer for 400 sec. Then, dissociation was measured for 500 sec. Dissociation constants (K_{D}) were analyzed with steady-state analysis using the HT 11.1 software provided with instruments. In case of NTD antibody (NTD Ab), anti-human IgG Fc capture (AHC) tips were functionalized with 5 μg/ml of NTD Ab in a kinetic buffer, and the same measurement procedure was applied. For comparison study of salt effect, kinetic buffers containing different NaCl concentrations (75 mM, 150 mM) were used.

### Preparation of Streptavidin Modified Polysaccharides

To immobilize the polysaccharides into nitrocellulose membrane, polysaccharides were conjugated to streptavidin by biotin-streptavidin interaction. Biotin modified polysaccharides were conjugated to streptavidin (1 mg/ml) with molar ratio of 4:1 (polysaccharides: streptavidin). After incubation for 1 hr at room temperature, the mixture solutions were purified to remove excess polysaccharides by using the amicon filter (30k, 50k, 100k) depending on the size of the polysaccharides.

### Preparation of Antibody Modified Gold Nanoparticles

For naked-eye detection, antibodies were conjugated to gold nanoparticles (AuNP) as a signaling probe. To prepare antibody-AuNP conjugates, NTD antibody (5 μl of 1 mg/ml), RBD antibody (5 μl of 1 mg/ml), ACE2 (10 μl of 0.63 mg/ml) were each added to 1 ml of AuNP (10 nm) with 0.1 ml of borate buffer (0.1 M, pH 8.5). After 1 hr incubation at room temperature, BSA (100 μl of 10 mg/ml) was introduced and incubated for 30 min to reduce the non-specific adsorption by blocking the surface of the gold nanoparticles. Then, the mixture solution was centrifuged at 22,000 g and 4° C. for 45 min. Supernatant was removed and AuNP solution was resuspended in 1 ml of BSA (1 mg/ml). Centrifugation and suspension process was repeated twice. Finally, antibody-AuNP conjugate was stored in the storage buffer (10 mM HEPES, 10 mM NaCl, 1 mg/ml BSA, pH 7.4) at 4° C.

For signal enhancement testing, gold nanoparticles were modified with horseradish peroxidase (HRP) conjugated NTD ab (NTD Ab-HRP). To prepare the gold nanoparticle modified with NTD-HRP (NTD-HRP-AuNP), 10 μl of NTD Ab-HRP (0.5 mg/ml) was added to the 1 ml of AuNP (10 nm) with 0.1 ml of borate buffer (0.1 M, pH 8.5). Then, the same procedure was utilized to prepare the NTD-HRP-AuNP.

### Preparation of Polysaccharides Based Lateral Flow Strip Biosensor

FIG. 4 shows the general design of the polysaccharides based lateral flow strip biosensor. Polysaccharides conjugated with streptavidin (1 mg/ml) and rabbit anti-human IgG (1 mg/ml) were dispensed on the nitrocellulose membrane (FF120HP). Dispensed nitrocellulose membrane was dried at 65° C. for 3 min. After drying, nitrocellulose membrane was blocked with a blocking buffer (1% BSA, 0.05% Tween-20 in 10 mM HEPES, 10 mM NaCl, pH 7.4). Finally, the sample pad (Whatman CF4 dipstick pad) and the absorbent pad (Whatman standard 17) were assembled onto the nitrocellulose membrane. Assembled strips were stored in the room temperature with desiccant before use.

### Screening Optimal Epitope and Buffer Using Lateral Flow Assay

To screen the optimal antibody for the sandwich-type detection of spike protein that will work along with polysaccharides, two antibodies which bind to different epitopes of spike protein (i.e., N-terminal domain binding antibody (NTD Ab) and receptor binding domain binding antibody (RBD Ab)), and ACE2 receptor were tested. The lateral flow strip for screening optimal epitope was prepared as described in the previous section. Dipstick method was used for testing all the lateral flow strips using 96 well plates. For the comparison study, 625 ng of SARS-CoV-2 spike was incubated with each signaling probe (20 nM) in the kinetic buffer (10 mM HEPES, 10 mM NaCl, Tween 20 0.05%, pH 7.4) for 5 min or 30 min at room temperature. Mixture solutions were loaded to the 96 well plate and prepared lateral strips were dipped for 20 min. After 20 min, red signals were observed by the naked eye and smartphone camera. Signals were quantitatively analyzed by imageJ software.

To test the effect of the NaCl on lateral flow assay, lateral flow strips, signaling probes, and SARS-CoV-2 spike were prepared by using HEPES buffers containing different concentrations of NaCl (10 mM, 75 mM, 150 mM). 25 μl of SARS-CoV-2 spike (25 μg/ml) and 25 μl of signaling probes (20 nM) prepared in HEPES buffers containing different concentrations of NaCl (10 mM, 75 mM, 150 mM) were incubated for 5 min. Then, the previously described dipstick method was used for the lateral flow assay.

### Selectivity and Sensitivity Analysis

For the selectivity test, 25 μl of each proteins SARS-CoV spike (50 μg/ml), MERS-CoV spike (50 μg/ml), ACE2 (50 μg/ml), human serum albumin (50 mg/ml), and bovine serum albumin (50 mg/ml), and the mixture of SARS-CoV spike (25 μg/ml), MERS-CoV spike (25 μg/ml), and SARS-CoV-2 spike (25 μg/ml) were incubated with 25 μl of signaling probe (NTD Ab-AuNP; 20 nM) for 5 min. Then, resulting solutions were loaded to the 96 well plate and lateral flow strip were dipped for 20 min.

For sensitivity test, 25 μl of various concentrations of spike (0, 0.39, 0.78, 1.56, 3.13, 6.25, 12.5. 25, 50 μg/ml) were incubated with 25 μl of signaling probe (NTD Ab-AuNP; 20 nM) for 5 min. Then, the same procedure of dipstick method was used for the lateral flow assay. The test line signals were quantitatively analyzed by imageJ software. The limit of the detection (LOD) was calculated by using blank+3 standard deviations.

### Signal Enhancement Analysis

For signal enhancement tests, a mixture of NTD Ab-AuNP and NTD Ab-HRP-AuNP were used as a signaling probe. Molar ratio of the mixture and reaction time was adjusted, and 1:1 molar ratio of NTD Ab-AuNP and NTD Ab-HRP-AuNP with 15 min reaction time were chosen for the signal enhancement testing. The assay was conducted in the buffer and spiked-in-human saliva condition. In the case of the buffer, 25 μl of various concentrations of spike (0, 0.05, 0.10, 0.20, 0.39, 0.78, 1.56, 3.13, 6.25, 12.5. 25, 50 g/ml) were incubated with 25 μl of signaling probe mixture (20 nM of NTD Ab-AuNP and NTD Ab-HRP-AuNP) for 5 min. Resulting solutions were loaded to the 96 well plate and prepared lateral strips were dipped for 20 min. Subsequently, 100 μl of AEC solution was introduced to enhance the signal for 15 min. For human saliva conditions, various concentrations of SARS-CoV-2 spike spiked in 1/50 diluted human saliva were used as a testing sample following the same test procedure, which was used in buffer condition. The test line signals were quantitatively analyzed by imageJ software. The limit of the detection (LOD) was calculated by using blank+3 standard deviations.

### Detection of Mutant Strain (SARS-CoV-2 Alpha, Beta, and Delta)

For the mutant strain testing, 25 μl of each proteins SARS-CoV-2 spike (50 μg/ml), Alpha strain spike-(50 μg/ml), Beta strain spike (50 μg/ml), and Delta strain spike (50 μg/ml) were incubated with 25 μl of signaling probe (NTD Ab-AuNP; 20 nM) for 5 min. Then, resulting solutions were loaded to the 96 well plate and lateral flow strip were dipped for 20 min. The test line signals were quantitatively analyzed by imageJ software.

### Abbreviations

RBD: Receptor Binding Domain
RBM: Receptor Binding Motif
NTD: N-terminal Domain
ACE2: angiotensin converting enzyme 2
HEP: heparin, (in this work: IdoA2S-GlcNS6S)
HS: heparan sulfate, (in this work: IdoA-GlcNAc6S)
DEX: dextran sulfate, (in this work: GlcA-GlcA2S4S)
CS: chondroitin sulfate, (in this work: GlcA2S-GalN4S6S)
APBS: Adaptive Poisson Boltzmann Solver
LFSA: Lateral-Flow Strip Assay
MD: molecular dynamics simulations
ESP: electrostatic potential
LOD: Limit of Detection
BSA: Bovine serum albumin
HSA: human serum albumin
HRP: horseradish peroxidase
AEC: 3-amino-9-ethylcarbazole

### REFERENCES

[1] S. Reitsma, D. W. Slaaf, H. Vink, M. A. M. J. van Zandvoort, M. G. A. oude Egbrink, Pflügers Arch.—Eur. J. Physiol. 2007, 454, 345.
[2] L. Mockl, Front. Cell Dev. Biol. 2020, 8, 253.
[3] S. Weinbaum, J. M. Tarbell, E. R. Damiano, Annu. Rev. Biomed. Eng. 2007, 9, 121.
[4] A. Varki, R. D. Cummings, J. D. Esko, P. Stanley, G. W. Hart, M. Aebi, A. G. Darvill, T. Kinoshita, N. H. Packer, J. H. Prestegard, R. L. Schnaar, P. H. Seeberger, Eds., Essentials of Glycobiology, Cold Spring Harbor Laboratory Press, Cold Spring Harbor (NY), n.d.
[5] E. Feyzi, T. Saldeen, E. Larsson, U. Lindahl, M. Salmivirta, J. Biol. Chem. 1998, 273, 13395.
[6] X. Han, P. Sanderson, S. Nesheiwat, L. Lin, Y. Yu, F. Zhang, I. J. Amster, R. J. Linhardt, Glycobiology 2020, 30, 143.
[7] P. Vongchan, M. Warda, H. Toyoda, T. Toida, R. M. Marks, R. J. Linhardt, Biochim. Biophys. Acta-Gen. Subj. 2005, 1721, 1.
[8] M. Warda, T. Toida, F. Zhang, P. Sun, E. Munoz, J. Xie, R. J. Linhardt, Glycoconj. J. 2006, 23, 555.
[9] B. Casu, in (Eds.: H. G. Garg, R. J. Linhardt, C. A. B. T.-C. and B. of H. and H. S. Hales), Elsevier Science, Amsterdam, 2005, pp. 1-28.
[10] M. Koehler, M. Delguste, C. Sieben, L. Gillet, D. Alsteens, Annu. Rev. Virol. 2020, 7, 143.
[11] L. J. Stroh, T. Stehle, Annu. Rev. Virol. 2014, 1, 285.
[12] A. J. Thompson, R. P. de Vries, J. C. Paulson, Curr. Opin. Virol. 2019, 34, 117.
[13] Y. Watanabe, J. D. Allen, D. Wrapp, J. S. Mclellan, M. Crispin, Science (80-.). 2020, 369, 330 LP.
[14] A. Shajahan, N. T. Supekar, A. S. Gleinich, P. Azadi, Glycobiology 2020, 30, 981.
[15] L. Casalino, Z. Gaieb, J. A. Goldsmith, C. K. Hjorth, A. C. Dommer, A. M. Harbison, C. A. Fogarty, E. P. Barros, B. C. Taylor, J. S. Mclellan, E. Fadda, R. E. Amaro, ACS Cent. Sci. 2020, 6, 1722.
[16] T. Sztain, S.-H. Ahn, A. T. Bogetti, L. Casalino, J. A. Goldsmith, R. S. McCool, F. L. Kearns, J. A. McCammon, J. S. Mclellan, L. T. Chong, R. E. Amaro, bioRxiv 2021, 2021.02.15.431212.
[17] D. Harmer, M. Gilbert, R. Borman, K. L. Clark, FEBS Lett. 2002, 532, 107.
[18] S. R. Tipnis, N. M. Hooper, R. Hyde, E. Karran, G. Christie, A. J. Turner, J. Biol. Chem. 2000, 275, 33238.
[19] I. Hamming, W. Timens, M. Bulthuis, A. Lely, G. Navis, H. van Goor, J. Pathol. 2004, 203, 631.
[20] E. P. Barros, L. Casalino, Z. Gaieb, A. C. Dommer, Y. Wang, L. Fallon, L. Raguette, K. Belfon, C. Simmerling, R. E. Amaro, Biophys. J. 2021, 120, 1072.
[21] P. Verdecchia, C. Cavallini, A. Spanevello, F. Angeli, Eur. J. Intern. Med. 2020, 76, 14.
[22] M. I. Zimmerman, J. R. Porter, M. D. Ward, S. Singh, N. Vithani, A. Meller, U. L. Mallimadugula, C. E. Kuhn, J. H. Borowsky, R. P. Wiewiora, M. F. D. Hurley, A. M. Harbison, C. A. Fogarty, J. E. Coffland, E. Fadda, V. A. Voelz, J. D. Chodera, G. R. Bowman, Nat. Chem. 2021, DOI 10.1038/s41557-021-00707-0.
[23] S. Y. Kim, W. Jin, A. Sood, D. W. Montgomery, O. C. Grant, M. M. Fuster, L. Fu, J. S. Dordick, R. J. Woods, F. Zhang, R. J. Linhardt, Antiviral Res. 2020, 181, 104873.
[24] T. M. Clausen, D. R. Sandoval, C. B. Spliid, J. Pihl, H. R. Perrett, C. D. Painter, A. Narayanan, S. A. Majowicz, E. M. Kwong, R. N. McVicar, B. E. Thacker, C. A. Glass, Z. Yang, J. L. Torres, G. J. Golden, P. L. Bartels, R. N. Porell, A. F. Garretson, L. Laubach, J. Feldman, X. Yin, Y. Pu, B. M. Hauser, T. M. Caradonna, B. P. Kellman, C. Martino, P. L. S. M. Gordts, S. K. Chanda, A. G. Schmidt, K. Godula, S. L. Leibel, J. Jose, K. D. Corbett, A. B. Ward, A. F. Carlin, J. D. Esko, Cell 2020, 183, 1043.
[25] Q. Zhang, C. Z. Chen, M. Swaroop, M. Xu, L. Wang, J. Lee, A. Q. Wang, M. Pradhan, N. Hagen, L. Chen, M. Shen, Z. Luo, X. Xu, Y. Xu, W. Huang, W. Zheng, Y. Ye, Cell Discov. 2020, 6, 80.
[26] L. Liu, P. Chopra, X. Li, K. M. Bouwman, S. M. Tompkins, M. A. Wolfert, R. P. de Vries, G.-J. Boons, bioRxiv 2021, 2020.05.10.087288.
[27] M. Yu, T. Zhang, W. Zhang, Q. Sun, H. Li, J. Li, Front. Mol. Biosci. 2021, 7, 490.
[28] R. S. Kalra, R. Kandimalla, Signal Transduct. Target. Ther. 2021, 6, 39.
[29] C. A. M. de Haan, Z. Li, E. the Lintelo, B. J. Bosch, B. J. Haijema, P. J. M. Rottier, J. Virol. 2005, 79, 14451 LP.
[30] A. Milewska, M. Zarebski, P. Nowak, K. Stozek, J. Potempa, K. Pyrc, J. Virol. 2014, 88, 13221 LP.
[31] I. G. Madu, V. C. Chu, H. Lee, A. D. Regan, B. E. Bauman, G. R. Whittaker, Avian Dis. 2007, 51, 45.
[32] R. Tandon, J. S. Sharp, F. Zhang, V. H. Pomin, N. M. Ashpole, D. Mitra, M. G. McCandless, W. Jin, H. Liu, P. Sharma, R. J. Linhardt, J. Virol. 2020, 95, DOI 10.1128/JVI.01987-20.
[33] A. M. Harbison, C. A. Fogarty, T. K. Phung, A. Satheesan, B. L. Schulz, E. Fadda, bioRxiv 2021, 2021.04.01.438036.
[34] G. Paiardi, S. Richter, M. Rusnati, R. C. Wade, arXiv.org 2021.
[35] C. A. M. de Haan, B. J. Haijema, P. Schellen, P. W. Schreur, E. the Lintelo, H. Vennema, P. J. M. Rottier, J. Virol. 2008, 82, 6078 LP.
[36] N. Vankadari, J. Phys. Chem. Lett. 2020, 11, 6655.
[37] A. Mohammad, E. Alshawaf, S. K. Marafie, M. Abu-Farha, J. Abubaker, F. Al-Mulla, Int. J. Infect. Dis. 2021, 103, 611.
[38] D. Wasik, A. Mulchandani, M. V. Yates, Biosens. Bioelectron. 2017, 91, 811.
[39] R. Jelinek, S. Kolusheva, Chem. Rev. 2004, 104, 5987.
[40] A. R. Griffith, C. J. Rogers, G. M. Miller, R. Abrol, L. C. Hsieh-Wilson, W. A. Goddard, Proc. Natl. Acad. Sci. 2017, 114, 13697 LP.
[41] B. H. Lee, S. H. Kim, Y. Ko, J. C. Park, S. Ji, M. B. Gu, Biosens. Bioelectron. 2019, 126, 122.
[42] S. H. Kim, J. Lee, B. H. Lee, C.-S. Song, M. B. Gu, Biosens. Bioelectron. 2019, 134, 123.
[43] G. A. Posthuma-Trumpie, J. Korf, A. van Amerongen, Anal. Bioanal. Chem. 2009, 393, 569.
[44] W. C. Mak, V. Beni, A. P. F. Turner, TrAC Trends Anal. Chem. 2016, 79, 297.
[45] N. H. Ahmad Raston, V.-T. Nguyen, M. B. Gu, Biosens. &amp; Bioelectron. 2017, 93, 21.
[46] J. Li, L. Jing, Y. Song, J. Zhang, Q. Chen, B. Wang, X. Xia, Q. Han, Nanoscale Res. Lett. 2018, 13, 296.
[47] K. M. Koczula, A. Gallotta, Essays Biochem. 2016, 60, 111.
[48] K. Hanack, K. Messerschmidt, M. Listek, 2016, pp. 11-22.
[49] T. R. Mercer, M. Salit, Nat. Rev. Genet. 2021, DOI 10.1038/s41576-021-00360-w.
[50] M. J. Mina, T. E. Peto, M. García-Fiñana, M. G. Semple, I. E. Buchan, Lancet 2021, 397, 1425.
[51] A. Crozier, S. Rajan, I. Buchan, M. McKee, BMJ 2021, 372, n208.
[52] E. Chautard, M. Fatoux-Ardore, L. Ballut, N. Thierry-Mieg, S. Ricard-Blum, Nucleic Acids Res. 2011, 39, D235.
[53] E. Chautard, L. Ballut, N. Thierry-Mieg, S. Ricard-Blum, Bioinformatics 2009, 25, 690.
[54] G. Launay, R. Salza, D. Multedo, N. Thierry-Mieg, S. Ricard-Blum, Nucleic Acids Res. 2015, 43, D321.
[55] O. Clerc, M. Deniaud, S. D. Vallet, A. Naba, A. Rivet, S. Perez, N. Thierry-Mieg, S. Ricard-Blum, Nucleic Acids Res. 2019, 47, D376.
[56] S.-J. Park, J. Lee, D. S. Patel, H. Ma, H. S. Lee, S. Jo, W. Im, Bioinformatics 2017, 33, 3051.
[57] S. Jo, K. C. Song, H. Desaire, A. D. Mackerell Jr., W. Im, J. Comput. Chem. 2011, 32, 3135.
[58] O. Guvench, E. Hatcher, R. M. Venable, R. W. Pastor, A. D. Mackerell, J. Chem. Theory Comput. 2009, 5, 2353.
[59] S. Jo, T. Kim, V. G. Iyer, W. Im, J. Comput. Chem. 2008, 29, 1859.
[60] J. Cladera, I. Martin, P. O'Shea, EMBO J. 2001, 20, 19.
[61] B. J. Connell, H. Lortat-Jacob, Front. Immunol. 2013, 4, DOI 10.3389/fimmu.2013.00385.
[62] H. A. Harrop, C. C. Rider, Glycobiology 1998, 8, 131.
[63] J. Hansen, A. Baum, K. E. Pascal, V. Russo, S. Giordano, E. Wloga, B. O. Fulton, Y. Yan, K. Koon, K. Patel, K. M. Chung, A. Hermann, E. Ullman, J. Cruz, A. Rafique, T. Huang, J. Fairhurst, C. Libertiny, M. Malbec, W. Lee, R. Welsh, G. Farr, S. Pennington, D. Deshpande, J. Cheng, A. Watty, P. Bouffard, R. Babb, N. Levenkova, C. Chen, B. Zhang, A. Romero Hernandez, K. Saotome, Y. Zhou, M. Franklin, S. Sivapalasingam, D. C. Lye, S. Weston, J. Logue, R. Haupt, M. Frieman, G. Chen, W. Olson, A. J. Murphy, N. Stahl, G. D. Yancopoulos, C. A. Kyratsous, Science (80-.). 2020, 369, 1010.
[64] R. Yan, Y. Zhang, Y. Li, L. Xia, Y. Guo, Q. Zhou, Science (80-.). 2020, 367, 1444.
[65] J. Lan, J. Ge, J. Yu, S. Shan, H. Zhou, S. Fan, Q. Zhang, X. Shi, Q. Wang, L. Zhang, X. Wang, Nature 2020, 581, 215.
[66] X. Chi, R. Yan, J. Zhang, G. Zhang, Y. Zhang, M. Hao, Z. Zhang, P. Fan, Y. Dong, Y. Yang, Z. Chen, Y. Guo, J. Zhang, Y. Li, X. Song, Y. Chen, L. Xia, L. Fu, L. Hou, J. Xu, C. Yu, J. Li, Q. Zhou, W. Chen, Science (80-.). 2020, 369, 650.
[67] A. Shrake, J. A. Rupley, J. Mol. Biol. 1973, 79, 351.
[68] M. Yuan, N. C. Wu, X. Zhu, C.-C. D. Lee, R. T. Y. So, H. Lv, C. K. P. Mok, I. A. Wilson, Science (80-.). 2020, 368, 630 LP.
[69] C. O. Barnes, A. P. West Jr., K. E. Huey-Tubman, M. A. G. Hoffmann, N. G. Sharaf, P. R. Hoffman, N. Koranda, H. B. Gristick, C. Gaebler, F. Muecksch, J. C. C. Lorenzi, S. Finkin, T. Hägglöf, A. Hurley, K. G. Millard, Y. Weisblum, F. Schmidt, T. Hatziioannou, P. D. Bieniasz, M. Caskey, D. F. Robbiani, M. C. Nussenzweig, P. J. Bjorkman, Cell 2020, 182, 828.
[70] M. Yuan, H. Liu, N. C. Wu, I. A. Wilson, Biochem. Biophys. Res. Commun. 2021, 538, 192.
[71] E. Jurrus, D. Engel, K. Star, K. Monson, J. Brandi, L. E. Felberg, D. H. Brookes, L. Wilson, J. Chen, K. Liles, M. Chun, P. Li, D. W. Gohara, T. Dolinsky, R. Konecny, D. R. Koes, J. E. Nielsen, T. Head-Gordon, W. Geng, R. Krasny, G.-W. Wei, M. J. Holst, J. A. McCammon, N. A. Baker, Protein Sci. 2018, 27, 112.
[72] N. A. Baker, D. Sept, S. Joseph, M. J. Holst, J. A. McCammon, Proc. Natl. Acad. Sci. 2001, 98, 10037 LP.
[73] M. Holst, F. Saied, J. Comput. Chem. 1993, 14, 105.
[74] M. J. Holst, F. Saied, J. Comput. Chem. 1995, 16, 337.
[75] M. Gasbarri, P. V'kovski, G. Torriani, V. Thiel, F. Stellacci, C. Tapparel, V. Cagno, Microorg. 2020, 8, DOI 10.3390/microorganisms8121894.
[76] C. J. Mycroft-West, D. Su, I. Pagani, T. R. Rudd, S. Elli, N. S. Gandhi, S. E. Guimond, G. J. Miller, M. C. Z. Meneghetti, H. B. Nader, Y. Li, Q. M. Nunes, P. Procter, N. Mancini, M. Clementi, A. Bisio, N. R. Forsyth, V. Ferro, J. E. Turnbull, M. Guerrini, D. G. Fernig, E. Vicenzi, E. A. Yates, M. A. Lima, M. A. Skidmore, Thromb. Haemost. 2020, 120, 1700.
[77] T. I. Přistoupil, M. Kramlová, J. Štěrbíková, J. Chromatogr. A 1969, 42, 367.
[78] J. Ceron, E. Lamy, S. Martinez-Subiela, P. Lopez-Jornet, F. Capela-Silva, P. Eckersall, A. Tvarijonaviciute, J. Clin. Med. 2020, 9, 1491.
[79] A. L. Wyllie, J. Fournier, A. Casanovas-Massana, M. Campbell, M. Tokuyama, P. Vijayakumar, J. L. Warren, B. Geng, M. C. Muenker, A. J. Moore, C. B. F. Vogels, M. E. Petrone, I. M. Ott, P. Lu, A. Venkataraman, A. Lu-Culligan, J. Klein, R. Earnest, M. Simonov, R. Datta, R. Handoko, N. Naushad, L. R. Sewanan, J. Valdez, E. B. White, S. Lapidus, C. C. Kalinich, X. Jiang, D. J. Kim, E. Kudo, M. Linehan, T. Mao, M. Moriyama, J. E. Oh, A. Park, J. Silva, E. Song, T. Takahashi, M. Taura, O.-E. Weizman, P. Wong, Y. Yang, S. Bermejo, C. D. Odio, S. B. Omer, C. S. Dela Cruz, S. Farhadian, R. A. Martinello, A. Iwasaki, N. D. Grubaugh, A. I. Ko, N. Engl. J. Med. 2020, 383, 1283.
[80] D. Zhou, C. Wu, Preprints 2020, DOI 10.20944/preprints202005.0192.v1.
[81] J. Corum, C. Zimmer, “Coronavirus Variants and Mutations,” can be found under https://www.nytimes.com/interactive/2021/health/coronavirus-variant-tracker.html, 2021.
[82] World Health Organization, “No Title,” can be found under https://www.who.int/en/activities/tracking-SARS-CoV-2-variants/, 2021.
[83] M. McCallum, A. C. Walls, K. R. Sprouse, J. E. Bowen, L. Rosen, H. V Dang, A. deMarco, N. Franko, S. W. Tilles, J. Logue, M. C. Miranda, M. Ahlrichs, L. Carter, G. Snell, M. S. Pizzuto, H. Y. Chu, W. C. Van Voorhis, D. Corti, D. Veesler, bioRxiv 2021, 2021.08.11.455956.
[84] S. M.-C. Gobeil, K. Janowska, S. McDowell, K. Mansouri, R. Parks, V. Stalls, M. F. Kopp, K. Manne, K. Saunders, R. J. Edwards, B. F. Haynes, R. C. Henderson, P. Acharya, bioRxiv Prepr. Serv. Biol. 2021, DOI 10.1101/2021.03.11.435037.
[85] N. Shiliaev, T. Lukash, O. Palchevska, D. K. Crossman, T. J. Green, M. R. Crowley, E. I. Frolova, I. Frolov, bioRxiv 2021, 2021.06.28.450274.
[86] C. Parolo, A. Sena-Torralba, J. F. Bergua, E. Calucho, C. Fuentes-Chust, L. Hu, L. Rivas, R. Álvarez-Diduk, E. P. Nguyen, S. Cinti, D. Quesada-González, A. Merkoçi, Nat. Protoc. 2020, 15, 3788.
[87] G. M. Morris, R. Huey, W. Lindstrom, M. F. Sanner, R. K. Belew, D. S. Goodsell, A. J. Olson, J. Comput. Chem. 2009, 30, 2785.
[88] O. Trott, A. J. Olson, J. Comput. Chem. 2010, 31, 455.
[89] F. Pedregosa, G. Varoquaux, A. Gramfort, V. Michel, B. Thirion, O. Grisel, M. Blondel, P. Prettenhofer, R. Weiss, V. Dubourg, J. Vanderplas, A. Passos, D. Cournapeau, M. Brucher, M. Perrot, E. Duchesnay, J. Mach. Learn. Res. 2011, 12, 2825.
[90] W. Humphrey, A. Dalke, K. Schulten, J. Mol. Graph. 1996, 14, 33.
[91] A. C. Walls, Y.-J. Park, M. A. Tortorici, A. Wall, A. T. McGuire, D. Veesler, Cell 2020, 181, 281.
[92] S. Bangaru, G. Ozorowski, H. L. Turner, A. Antanasijevic, D. Huang, X. Wang, J. L. Torres, J. K. Diedrich, J.-H. Tian, A. D. Portnoff, N. Patel, M. J. Massare, J. R. Yates, D. Nemazee, J. C. Paulson, G. Glenn, G. Smith, A. B. Ward, Science (80-.). 2020, 370, 1089 LP.
[93] M. H. M. Olsson, C. R. Søndergaard, M. Rostkowski, J. H. Jensen, J. Chem. Theory Comput. 2011, 7, 525.
[94] C. R. Søndergaard, M. H. M. Olsson, M. Rostkowski, J. H. Jensen, J. Chem. Theory Comput. 2011, 7, 2284.
[95] G. Madhavi Sastry, M. Adzhigirey, T. Day, R. Annabhimoju, W. Sherman, J. Comput. Aided. Mol. Des. 2013, 27, 221.
[96] J. C. Phillips, R. Braun, W. Wang, J. Gumbart, E. Tajkhorshid, E. Villa, C. Chipot, R. D. Skeel, L. Kalé, K. Schulten, J. Comput. Chem. 2005, 26, 1781.
[97] J. C. Phillips, D. J. Hardy, J. D. C. Maia, J. E. Stone, J. V Ribeiro, R. C. Bernardi, R. Buch, G. Fiorin, J. Hénin, W. Jiang, R. McGreevy, M. C. R. Melo, B. K. Radak, R. D. Skeel, A. Singharoy, Y. Wang, B. Roux, A. Aksimentiev, Z. Luthey-Schulten, L. V Kalé, K. Schulten, C. Chipot, E. Tajkhorshid, J. Chem. Phys. 2020, 153, 44130.
[98] O. C. Grant, D. Montgomery, K. Ito, R. J. Woods, Sci. Rep. 2020, 10, 14991.
[99] R. Farid, T. Day, R. A. Friesner, R. A. Pearlstein, Bioorg. Med. Chem. 2006, 14, 3160.
[100] W. Sherman, H. S. Beard, R. Farid, Chem. Biol. Drug Des. 2006, 67, 83.
[101] W. Sherman, T. Day, M. P. Jacobson, R. A. Friesner, R. Farid, J. Med. Chem. 2006, 49, 534
[102] G. van Meer, D. R. Voelker, G. W. Feigenson, Nat. Rev. Mol. Cell Biol. 2008, 9, 112.
[103] W. L. Jorgensen, J. Chandrasekhar, J. D. Madura, R. W. Impey, M. L. Klein, J. Chem. Phys. 1983, 79, 926.
[104] J. Huang, S. Rauscher, G. Nawrocki, T. Ran, M. Feig, B. L. de Groot, H. Grubmüller, A. D. Mackerell, Nat. Methods 2017, 14, 71.
[105] J. Huang, A. D. Mackerell, J. Comput. Chem. 2013, 34, 2135.
[106] R. E. Amaro, A. J. Mulholland, Comput. Sci. Eng. 2020, 22, 30.
[107] A. J. Mulholland, R. E. Amaro, J. Chem. Inf. Model. 2020, 60, 5724.

### Example 2

Heparan sulfate conjugated with streptavidin was immobilized onto a nitrocellulose membrane via non-specific adsorption. A CD4 receptor, which is known for cell entry receptor for HIV, was conjugated onto gold nanoparticles (AuNP) to provide a detection agent.

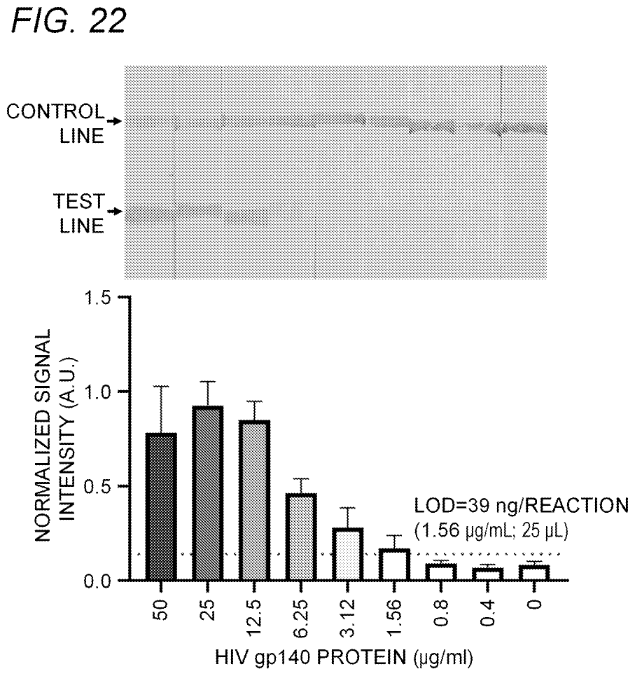

To determine the sensitivity of the HSPG or GAGs based lateral flow assay, different concentrations of the HIV gp140 protein (i.e., an envelope protein on the HIV from group M, subtype CRF07_BC) was tested in buffer conditions. The tested concentration range was 0 μg/ml to 50 μg/ml of monomeric HIV gp140 protein. The limit of the detection of this HSPG or GAGs based lateral flow assay was 39 ng/reaction (1.56 μg/ml; 25 μl). The results are shown in FIG. 22, which provides an image of the tests strips with the tested concentrations of 50 μg/ml to 0 μg/ml (from left to right as shown in the below graph) and a graph providing the normalized signal intensity for the tested concentrations. In a subsequent assay, the limit of the detection of this HSPG or GAGs based lateral flow assay, with signal enhancement as shown in FIG. 40, panel B, was 5 ng/reaction (0.2 μg/ml; 25 μl).

### Example 3

To test the feasibility of sensor in real sample conditions, sensitivity was tested on the human serum condition. The tested concentration range was 0 μg/ml to 50 μg/ml of monomeric HIV gp140 protein. The limit of detection of this HSPG or GAGs based lateral flow assay was 78 ng/reaction (3.12 μg/ml; 25 μl) in human serum. To determine the sensitivity of the HSPG or GAGs based lateral flow assay to trimeric gp140 protein, different concentrations of the HIV gp140 protein (i.e., an envelope protein on the HIV from group M, subtype C). The tested concentration range was 0 μg/ml to 50 μg/ml of trimeric HIV gp140 protein. The limit of the detection of this HSPG or GAGs based lateral flow assay was 78 ng/reaction (3.12 μg/ml; 25 μl). Furthermore, various subtype detection of this this HSPG or GAGs based lateral flow assay was tested with trimeric HIV gp140 protein (i.e., an envelope protein on the HIV from group M, subtype C, subtype A, and subtype B). It could detect all subtypes tested.

### Example 4

Several SARS-CoV-2 variants of concern (VOCs) have emerged thus far over the course of the COVID-19 pandemic including Alpha, Beta, Gamma, Delta, and Omicron, the latter with its own sub-lineages BA.1, BA.2, BA.3, BA.4, and BA.5.¹⁻³ Each of these VOCs are characterized by key mutations throughout the genome.¹ The SARS-CoV-2 viral envelope is studded with approximately 30 homotrimer glycoproteins, called spike proteins, which play the primary role in initiating host-cell entry via their receptor binding domains (RBDs). Genomic mutations to the spike protein sequence have been implicated in increasing infectivity and/or immune escape.⁴-8 The Alpha, Beta, Delta, and Omicron BA. 1 genomes, for example, contain 8, 8, 9, and 34 mutations in their spike mRNA sequences relative to the original “wild type” (WT) 2019 strain⁹-13 (Table 2).

Due to the high number of sequence mutations characteristic of the Omicron variant, its initial PCR detection was abrogated, and its rapid detection was imparid.^{14,15} Rapid antigen detection was also impaired for initial Omicron variants even though these commercially available kits detect nucleocapsid proteins which incur a lower rate of mutation.^{14,15} Even more, it has been recently reported that newer subvariants of Omicron, particularly BA.5, could completely escape from detection in current rapid kits.¹⁶ Therefore, the FDA has recently recommended (Aug. 11, 2022) repeated testing within 48 hours to overcome the reduced sensitivity of the rapid kits.¹⁷ Rapid antigen detection of variant spike proteins is even more challenging considering the spike genome's high mutation rate, often necessitating re-screening of spike antibodies, a step which can cause significant lag time behind the emergence of new variants¹⁸⁻²⁰

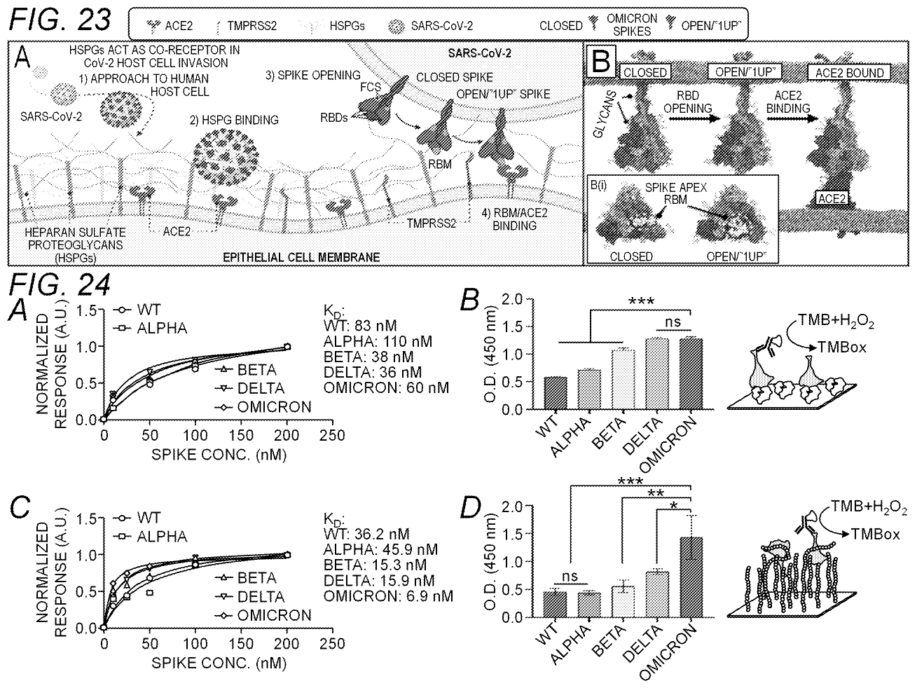

Elucidating the factors driving SARS-CoV-2 evolution affecting spike binding kinetics and stability at the host-cell surface will help predict further mutations or gains of function, as well as aid in developing variant-specific antiviral therapies and better antigen testing platforms. As cellular invasion by SARS-CoV-2 may involve at least 3 distinct cell surface glycoproteins (HSPG, ACE2, TMPRSS2), the spike mutations might alter its interactions with any or all of them (FIG. 23).²¹-26 Upon approach to the cell (Step 1 in FIG. 23, Panel A), the SARS-CoV-2 virion first encounters the glycocalyx, a dense sugary matrix extending from the epithelial cell membrane.^{27,28} Heparan sulfate proteoglycans (HSPGs), key components of the glycocalyx, are known to serve as attachment factors for many viruses and likely to make first contact with SARS-CoV-2.²⁹⁻³¹ HSPGs contain long, intrinsically disordered protein backbones decorated with longer (40-400 monomeric units) poly-sulfated and densely negatively charged glycosaminoglycans (GAGs).³² Heparan sulfate (HS) itself is biosynthesized natively in repeating dimeric units of N-acetyl-D-glucosamine and D-glucuronic acid; post-processing enzymes then add sulfate groups to certain positions along an HS sequence, and epimerization enzymes may convert some D-glucuronic acid monomers to L-iduronic acid. Neither sulfation nor epimerization reactions go to completion, thus there exist locally controlled regions of high/low sulfation/epimerization proportions further contributing to vast degree of glycocalyx heterogeneity. HS regions with particularly high proportions of sulfation and L-iduronic acid are referred to as “heparin-like” domains, calling upon their similarity to short-chain medicinal heparin (HEP), which is almost completely sulfated and epimerized.³²

Several studies in early 2020 first illustrated that the SARS-CoV-2 spike protein, particularly the spike RBD, can bind to HS and/or HEP,³⁴⁻³⁷ and Clausen et al. showed that cellular HS was required for SARS-CoV-2 host-cell invasion.²⁷ With these prior results, the second step in the viral invasion process comes into focus, as shown in FIG. 23, Panel A: Binding of the virion to HSPGs, likely through direct spike-HS interactions as shown by us³⁸ and others^{27,28,34-36,39-48} This step may increase virion residence time at the host-cell surface, thereby increasing the likelihood of encountering angiotensin converting enzyme 2 (ACE2), SARS-CoV-2's primary host-cell receptor.^{44,45,49} The spike protein binds to ACE2 via a highly specialized interface within the Receptor Binding Domain (RBD) called the Receptor Binding Motif (RBM).⁵⁰⁻⁵⁵ Throughout much of the spike protein lifecycle, each of the spike's RBDs (1 RBD per spike protomer, 3 RBDs per spike protein, 1 RBM per RBD, 3 RBMs per spike protein) occupies a “closed” or “down” conformational state wherein spike RBMs are largely shielded from recognition.⁵⁶⁻⁵⁹ Thus, before spike-ACE2 binding can occur, at least one of these RBDs needs to emerge from its down/“shielded” state to an “up”/“open” state, to expose its RBM (FIG. 23, Panel B).^{56,57} While this step (FIG. 23, Panel B) can occur anytime along the spike lifecycle, Clausen et al report that short chain HEP is capable of inducing spike RBDs to move into the up state, suggesting that RBD opening could be induced after spike binding to HS and/or HEP within the glycocalyx.²⁷ With the RBM exposed, ACE2 can bind, further stabilizing the virion at the host-cell surface. Finally, with the spike protein immobilized by HS and/or ACE2, TMPRSS2 can cleave the S1 from the S2 spike domain at the Furin Cleavage Site (FCS).60-64 After cleavage, the freed S1 domain peels off from the S2, revealing the spike's fusion peptide (FP) which then penetrates the host cell membrane and initiates membrane fusion 65-67

Recent studies have demonstrated that the Omicron SARS-CoV-2 virion relies less on membrane fusion as catalyzed by TMPRSS2 S1/S2 cleavage than earlier strains and rather more on pinocytosis. Indeed, syncytial formation-neighboring SARS-CoV-2 infected cells fusing together, a phenomenon indicative of TMPRSS2 activity—was reduced for Omicron infected tissues.^{68,69} Furthermore, infection of TMPRSS2 knock-out cells by SARS-CoV-2 was increased for Omicron relative to other VOCs.^{69,70} This mechanistic change could be due to sequence mutations near the Omicron spike's FCS, causing decreased recognition by TMPRSS2.⁶⁸⁻⁷⁰

Furthermore, recent research suggests that mutations in the Delta and Omicron RBMs result in altered binding affinities to ACE2 compared to WT.²¹-26 Several past works have identified that charge-charge interactions heavily stabilize the WT spike-ACE2 interface,⁷¹ and that increasing spike charge over the course of SARS-CoV-2 evolution potentially increases electrostatic recognition of ACE2 at long range and increases immune escape,⁴⁷ especially for Omicron, as its spike's RBM is more positively charged than other spikes.⁴⁸

While the role of evolving positive charge on spike proteins has begun to be unraveled for ACE2,^{21-26,47,48} this investigation was done in isolation from its required co-factor, HS.⁷¹ As HS is a long, negatively charged polysaccharide, the growing positive charge on spiked virions, especially for Omicron, is expected to impact the stability of interactions with the HS-rich glycocalyx. Additionally, since HS was previously shown to stabilize spike-ACE2 interactions, an altered affinity to HS may in turn also affect binding to ACE2.

Here, we probe the secondary and ternary complex interactions between HS, ACE2, and the spike proteins of WT, Alpha, Beta, Delta, and Omicron SARS-CoV-2 variants. We propose how the positively charged Omicron spike may unlock a critical HS/ACE2 synergy. By harnessing the power of the primary and secondary cell receptors in the glycocalyx, we show how the performance of HEP/HS anchored test strips can co-evolve with the SARS-CoV-2 genome for robust and rapid sensing of Omicron SARS-CoV-2. Finally, given that our synthetic glycocalyx test strips represent a minimal model for the cell surface, we discuss the potential implications our results may have on understanding SARS-CoV-2 infection dynamics at large.

### Spike Mutations Exhibit Increased Binding to ACE2 & Heparin

Previous work has demonstrated mixed results with respect to relative binding affinities between ACE2 and different spike protein sequences. Some groups report the Omicron spike protein binds to ACE2 with highest affinity, while others report there is no significant difference in binding affinity between all variants.²¹⁻²⁶ Herein, we have used BLI, ELISA, and molecular dynamics (MD) simulations to estimate relative binding affinities between WT, Alpha, Beta, Delta, and Omicron BA.1 spike proteins to ACE2. BLI results indicate similar binding affinities within nanomolar range for all protein complexes, with Beta and Delta having the highest affinity, followed by Omicron BA.1, WT, and finally Alpha (FIGS. 23, Panel A and 31). ELISA results show a clearer trend: Delta and Omicron spikes similarly show higher affinity to ACE2 than all other spike proteins (FIG. 23, Panel B). Our BLI and ELISA results were consistent with previous reports showing affinities of Delta and Omicron similarly increased compared to WT.^{21,22,25,26}

We also performed MD simulations of WT, Delta, and Omicron RBDs bound to ACE2. From these simulations we see interactions at the RBD/ACE2 interface that may explain increased binding affinity between Omicron and WT. Barros et al. discuss the relative contact frequencies of three subregions within the RBM to ACE2: the L3 loop, the central beta-strands, and the right-hand loops.⁵³ They demonstrate that contacts formed between the central beta-strands and ACE2 are the strongest and maintain almost completely during their 1 us of conformational sampling, whereas contacts formed between the L3 loop and right-hand loops to ACE2 are weaker, measured by decreased contacting frequency. From our MD simulations we see the Omicron RBM, with 10 total mutations, both strengthens interactions to ACE2 within the central beta strands (addition of salt bridge between Q493R to ACE2's E35) but also increases stabilization within the weaker right-handed loops through an aromatic interaction (N501Y pi-stacking with ACE2's Y41), and a tight hydrogen bonding network (Y505H tightly supporting a hydrogen bond between RBD's Y495 and ACE2's K353). These interactions, as also elucidated by Han et al.,⁷² potentially explain the strengthening of affinity between Omicron spike and ACE2 relative to the WT.

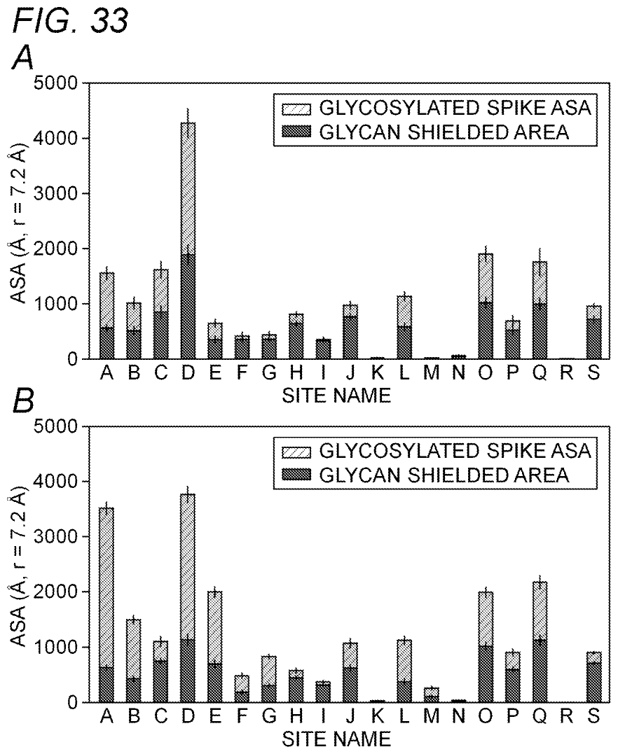

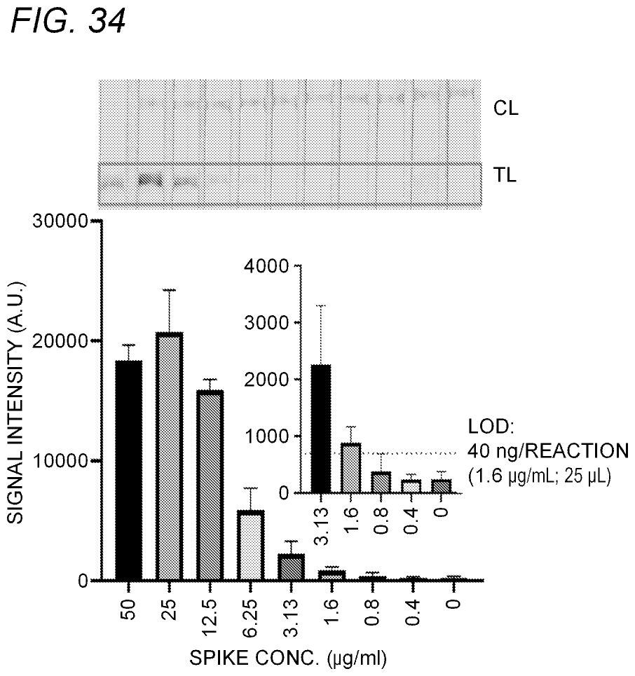

As interactions between the SARS-CoV-2 spike protein and HS in the glycocalyx is a requisite step to binding ACE2,²⁷ we also investigated binding affinity between spike proteins and HEP. BLI results illustrate an increase in affinity between HEP and Omicron spike over Delta and other VOC spikes (FIGS. 23, Panel C and 32). This result is also confirmed with ELISA as Omicron spikes bind to HEP with highest affinity, followed by Delta, then Beta, then Alpha and WT at similar affinity (FIGS. 23, Panel D). To probe differences in affinity to HEP at the molecular scale, we conducted extensive ensemble-based docking studies with AutoDock Vina^{73,74} to predict HEP binding modes to WT, Delta, and Omicron spikes in closed and open conformational states. From ˜28,800 binding modes, we clustered to identify 15 HEP binding “hotspots” on the spike surface. Relative affinities and populations of HEP binding modes at each of these sites were similar across the three spike variants (FIG. 33). In past work, we predicted 3 sites of high importance for interaction between the spike protein and HS: an RBD cleft site, an RBD patch site, and the FCS.³⁸ Current ensemble-based docking simulations have confirmed the presence of these sites on WT and variant spikes and indicate there are no significant differences in binding affinities or number of binding modes in these sites between spike variants (FIG. 33). To determine the degree to which induced-fit effects within the RBD Cleft, RBD Patch, FCS, and potential binding at the RBM could impact affinity, we then conducted targeted flexible docking studies with HEP and HS tetramers at each of these sites across WT, Delta, and Omicron variants with Schrödinger's Induced Fit Docking protocol.⁷⁵⁻⁷⁹ Again, there were no significant differences between average predicted binding energies for HEP or HS tetramers at each of these sites, across the three spike variants, even with global (MD enabled ensemble docking) and local (Schrödinger IFD) protein flexibility incorporated (data not shown). This likely indicates that once a HS/HEP fragment finds a site on the spike surface, it is flexible enough to accommodate sequence mutations and maintain affinity at the surface. These docking results suggest that the increased binding affinity between HEP and SARS-CoV-2 Delta and Omicron spikes relative to WT, as observed with BLI and ELISA, most likely do not originate from site-specific changes.

### Evolution of Positive Charges on Spike Protein Enhances Rate of Binding to ACE2 & Heparin

As previously noted by us^{49,80} and others⁸¹, the spike protein is becoming more positively charged with each emerging VOC spike sequence. The total formal charge of the trimeric WT spike head domain (residues 13 to 1140) at pH 7.4 is +3, Alpha is +6, Beta+15, Delta +18, and Omicron BA.1+24; thus, the Alpha, Beta, Delta, and Omicron BA.1 spike protein sequences follow a trend of +3, +12, +15, and +21 in charge relative to the WT. Several glycans on the spike ectodomain are also shown to be sialylated. While a complete differentiation of glycan sialylation rate per spike sequence is far beyond the scope of this work, it is important to estimate the relative contribution of sialic acids to total spike head domain charge. Assuming a glycoprofile consistent with models from Casalino et al.⁵⁶ (14 sialic acids) and described by Watanabe et al.,⁸² the total formal charge of the trimeric WT spike head domain with glycans is −11, Alpha is −8, Beta +1, Delta +4, and Omicron BA.1+10 (Table 2). From this accounting of charge, it's clear to see that due to mutations in spike sequence, the spike protein head domains are increasing in total charge.

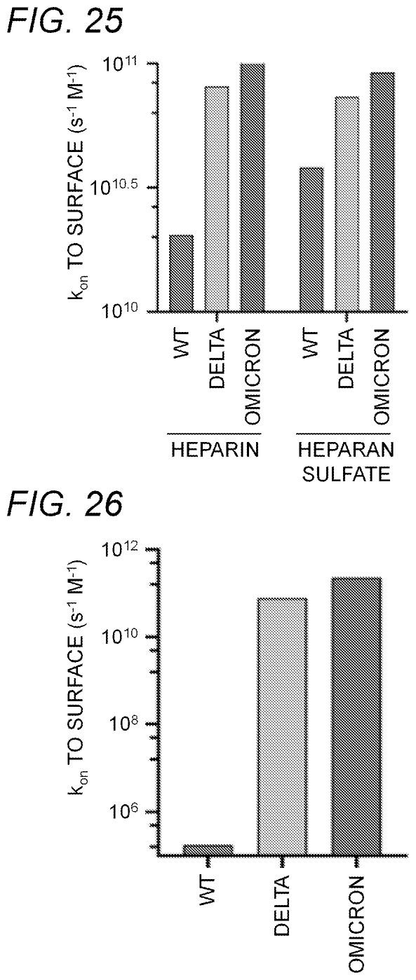

To investigate the effects of spike total charge on HEP binding, we used Brownian dynamics (BD)⁸⁴ simulations with Browndye⁸⁵ to calculate rate constants (kₒₙ) to a “b surface”, wherein the center of mass of a receptor molecule of interest defines the center of a sphere with a “b-radius” (FIG. 25). The receptor and ligand molecules, each containing partial atomic charges, approach one another from infinite space. In such a model, a kₒₙ between two molecules attaining an intermolecular distance less than the b-radius is largely driven by charge-charge interactions and can thus be solved numerically using the Smoluchowski equation.⁸⁶ These results provide insight into long-range electrostatic interactions between molecules. We observe a dramatic increase in kₒₙ to the b-surface between a HEP tetramer (charge −8) and WT, Delta, and Omicron spike proteins, 2×10¹⁰ M⁻¹ s⁻¹, 8×10¹⁰ M⁻¹ s⁻¹, 1×10¹¹ m⁻¹ s⁻¹, respectively, (FIG. 25). a similar trend is observed for the kₒₙ to the b-surface between a model HS tetramer (charge-4) and WT, Delta, and Omicron spikes (FIG. 25). Additionally, seeing as the kₒₙ to the b-surface calculated for HS to WT was higher than that for HEP to WT, we predict that HS, owing to its decreased sulfation and charge, is likely to find and bind more quickly to WT spike surface than fully sulfated HEP domains. These results indicate that optimized long-range electrostatic interactions via spike mutations could dramatically impact the rate of SARS-CoV-2 viral approach to the glycocalyx (Step 1 in FIG. 23, Panel A). Together with our docking results, which predicted very little difference between VOC spikes in HEP binding affinities at HEP-binding hotspots, the BD results illustrate that increased affinities between HEP and Delta/Omicron spikes relative to WT, as seen by BLI and ELISA, may be due to kinetic selection allowing for increased encounters rather than site-specific differences in binding affinity. Furthermore, due to Delta's and Omicron's apparent kinetic preference for HEP over HS, as opposed to WT, newer variants likely demonstrate an increased selectivity for the more densely sulfated/charged heparin-like domains.

Considering that ACE2's dimeric exodomain has a total charge at pH 7.4 of-42 (total formal charge with glycans and at pH 7.4,-54)⁵³ rates to ACE2, driven by long-range electrostatic interactions, may also be affected by increasing spike protein charge. Therefore, we also calculated rate constants (kₒₙs) to b-surfaces between the ACE2 ectodomain and WT, Delta, and Omicron spike proteins. Interestingly, we see six orders of magnitude increase in kₒₙ between WT and Delta spikes to ACE2, followed by a one order of magnitude increase in kₒₙ between Delta and Omicron spikes to ACE2 (FIG. 26). As with the HS/HEP results, the increasing total charge of spike proteins may strengthen long-range electrostatic interactions to negatively charged ACE2 (−42). Additionally, recall that binding affinities between SARS-CoV-2 VOC spikes and ACE2 are increasing (decreasing K_{D}), but only moderately (FIG. 24, Panels A and B). In sum, these BD results for HEP, HS, and ACE2 all point to kinetic fitness within the negatively charged glycocalyx as a potential underlying evolutionary pressure driving SARS-CoV-2 spike sequence adaptation.

### Remapping of Positive Charge Distribution on Omicron Surface Maximizes Heparin/ACE2 Synergy

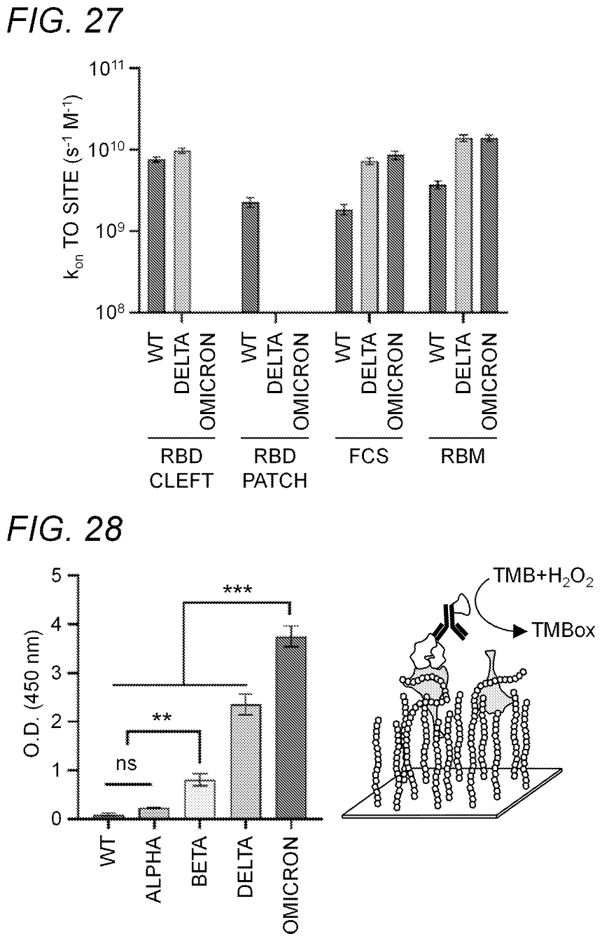

Dynamically averaged electrostatic potential maps demonstrate that the spike sequence mutations in the Delta and Omicron variants increase the total spike charge as well as redistribute surface patches of positive and negative charge (data not shown). As a result, the site of first contact between HS/HEP and spike surface, i.e., a nucleation site for HS/HEP long-chain binding to the spike surface, could be altered on a per-VOC basis. To probe these changes, we again used BD simulations to investigate the rate of HS and HEP tetramer association, this time specifically to the RBM, RBD cleft, RBD patch, and the FCS sites. We find that HEP tetramers associate differentially to spike surface sites due to mutations at each site (FIG. 27). Upon approaching a WT spike protein, our kinetic experiments indicate that HEP tetramers are most likely to associate with the RBD Cleft site first, followed by the RBM, the RBD Patch, and finally the FCS. However, upon approaching a Delta spike protein, HEP tetramers are most likely to find the RBM first, followed closely by the RBD Cleft, and finally the FCS, with no observed transitions into the RBD Patch. Similarly, when encountering Omicron spike proteins, HEP tetramers are most likely to first find and bind the RBM, followed by the FCS, with no observed transitions to the RBD Cleft or Patch sites. These results indicate that redistribution of positive charges, especially for Omicron spikes, might cause a competition between HEP and ACE2 binding on RBM site of spike protein. However, at the cell surface, ternary complex formation between HS, spike, and ACE2 (FIG. 28) is potentially required for stabilization of the spike/ACE2 interface.²⁷ Thus, we conducted ternary complex ELISA to identify whether HEP and ACE2 compete with one another for spike binding on a per-VOC basis. Strikingly, we observed a significant increase in the affinity of the Omicron spike/HEP/ACE2 ternary complex over all other variants, including Delta. This result potentially indicates that later VOC spikes, especially Omicron, unlock a cooperative synergy between HS and ACE2.

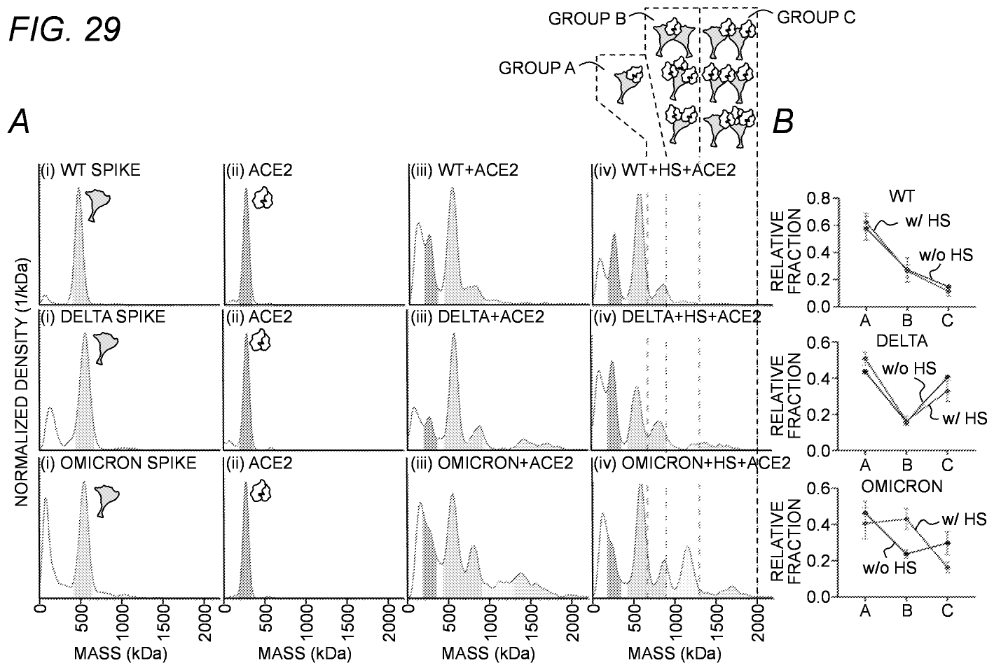

To investigate further the synergistic formation of ternary spike/HS/ACE2 complexes we used Mass Photometer (MP) and compared the effect of HS on formation of ternary complexes for WT, Delta, and Omicron spike proteins in the presence of dimeric ACE2 by measuring the mass distributions (FIG. 29). The mass of each trimeric spike protein was measured to be around 560 kDa, and the mass of dimeric ACE2 was 240 kDa. Given that spike likely first encounters the extended tendrils of HS upon approach to the human host-cell, we sequentially added first HS and then ACE2 to spike protein samples to mimic conditions at the cell surface. Co-incubation of spike with just ACE2 generated mass peaks for WT, Delta, and Omicron spike around 800 kDa, followed by signal density in higher mass ranges indicating that spike and ACE2 are interacting to form complexes at varying stoichiometric ratios (FIG. 29, Panel A (iii)). Interestingly, while incubating spike and ACE2 with HS yielded very little differences in MP spectra for WT and Delta spikes compared to no-HS conditions, Omicron showed a significant increased population around 1200 kDa under these testing conditions (FIG. 29, Panel A (iv)), suggesting it plays a role in stabilizing a ternary spike/HS/dACE2. To assign possible stoichiometries to the emerging 1200 kDa complex, we must understand the structural requirements for such assembly. Spike protein binding to ACE2 requires at least one spike RBD to be in the up conformation and a successful binding event between the two proteins is canonically considered as occurring between one ACE2 and one 1-up spike. However, a spike protein with three RBDs in the up-state could accommodate binding of up to three dACE2 ectodomains and dimeric ACE2 could itself accommodate binding of up to two spike proteins.^{53,87} Additionally, while dimeric ACE2 was used in this work, without the presence of B⁰AT-1 and its corresponding stabilization of the ACE2 interfacial neck domain, ACE2 in solution could exhibit more flexibility and adopt dual RBD binding modes as described by Xiao et al.⁸⁸ While all such complexes are likely biologically relevant, the degree to which, and by what mechanism(s), spike and ACE2 form such complexes of “intermediate” stoichiometry is an open question. Thus, to parse our current Mass Photometry results, we have enumerated several configurations of spike-ACE2 complexes (illustration within FIG. 29, Panel A) and divided such complexes into three groups based on their expected mass range: A (650-900 kDa), B (900-1300 kDa), C (1300-2000 kDa). To compare the change in ternary complex distribution with or without HS, the fraction of each group (denoted as A, B, C) was calculated for each spike protein (Tables 5-7). As shown in FIG. 29, Panel B, although the addition of HS slightly increased the population of group A type complexes for WT and Delta spikes, there was no significant change in degree of complex formation for types A, B, or C for WT and Delta spikes. However, Omicron spikes showed increased proportions of type B complexes in the presence of HS (FIG. 29, Panel B). Considering that HS may stabilize spike RBDs in their up conformation, as reported by Clausen et al.,²⁷ binding of multiple HS fragments to the Omicron spike could serve to recruit additional nearby ACE2s for binding, thereby increasing the population of group B type complexes (Spike: ACE2=1:2, 1:3). Additionally, an increased proportion of group B could also stem from one dimeric ACE2 binding two trimeric spike proteins, an interaction which could easily be facilitated by long chains of HS either binding one or both spike proteins. ACE2 bridging multiple spike proteins is likely an important factor governing complex formation at the cell surface, as the local concentration of the spike protein is likely to be higher than the local concentration of ACE2 and HS is further likely to “hold” or cluster spikes near ACE2 in preparation for binding of the ternary complex.

To summarize all results presented thus-far: (1) binding affinity between SARS-CoV-2 VOC spikes to ACE2 are moderately increasing over the variant timeline, (2) site specific affinities between SARS-CoV-2 VOC spikes to HEP dimers and tetramers have not changed significantly over the variant timeline but (3) binding affinity between spike VOCs and long-chain HEP has increased over the variant timeline, (4) increasing total spike charge over the variant timeline may be increasing rates of HEP/HS/ACE2 to spike surfaces, (5) charge redistribution on the spike surface over the variant timeline may be altering HEP/HS nucleation sites in the context of long-chain binding interactions, and finally (6) Omicron has a particular ability to unlock a key HS/ACE2 synergy by increasing proportions of 1:2 and 1:3 spike: ACE2 complexes. At the cell surface, an individual spike glycoprotein will likely encounter both HS and ACE2. In what order, and by what mechanism(s) does the spike glycoprotein interact with and exploit the native functions of HS and ACE2 to enter the human host-cell, and how do mutations to the spike sequence affect this mechanism remain outstanding questions. As illustrated in FIG. 23, for a spike-ACE2 binding event to occur, the spike's RBM needs to be sufficiently exposed, which only occurs when at least one of the spike protein's RBDs moves from a “down”/“shielded” state to the “up”/“exposed” state. Clausen et al. report that short chain HEP can increase proportions of ACE2s bound to the spike protein, suggesting HEP can facilitate RBD opening and ACE2 binding. Based on our results, we hypothesize that as the total formal charge of the spike protein increases so does the spike's fitness for moving through and interacting with the negatively charged glycocalyx and ACE2 as shown in BLI, ELISA, and BD results. Moreover, as the spike approaches the glycocalyx, certain sites (i.e., the RBM and FCS) on VOC spikes may find and bind to HS more quickly than to others due to redistribution of charges on the spike surface. For example, in the case of the Omicron spike protein, given the rate constant for HEP binding is fastest to exposed RBMs, HS/HEP could increase the local concentration of 1 up, 2 up, and 3 up Omicron spike proteins directly at the cell surface. While bound to the Omicron spike protein, HS would thereby stabilize spike in an attack-ready conformation while ACE2 arrives on the cell surface below. ACE2 could eventually displace HS from the RBM, which could in turn shift to one of the many other GAG-hotspots on the spike surface, including the FCS. In this fashion, the Omicron spike protein's RBM could be capitalizing on HS/HEP's capacity for kinetic selection, thereby increasing the localized concentration of ACE2-ready binding partners at the cell surface. Additionally, quick and stable binding of HS to Omicron spike's FCS could also correspond to decreased TMPRSS2 cleavage efficiency shown by other groups^{64,68-70} as HS may shield the FCS from TMPRSS2 recognition. Taken together, we suggest a mechanism by which SARS-CoV-2 variants evolve to better bind the co-receptor glycocalyx HS, which indirectly enhances its chances to bind and the stability of its interactions with the primary receptor, ACE2.

### GlycoGrip is a Minimal Model for the Host-Cell Surface and Signals Effectively for Evolved Variants

As discussed in the introduction, maintaining robust testing via rapid antigen and PCR detection platforms becomes a challenge during an actively progressing public health crisis such as the COVID-19 pandemic. Recently, we showed that the interaction between the host-cell surface glycopolymers and the spike glycoprotein can be exploited in a sandwich-style lateral flow strip-based assay (LFSA) to tackle this exact challenge.⁸⁹ Our sensor termed GlycoGrip, was inspired by the interaction between SARS-CoV-2 virions and the glycocalyx. GlycoGrip uses long-chain Heparin (HEP) to capture, and Au-nanoparticle conjugated anti-spike antibodies to signal for the presence of SARS-CoV-2 spike proteins. However, our 1st generation GlycoGrip, a.k.a. GlycoGrip1.0, utilized an N-terminal domain (NTD) based signaling antibody which had potential for decreased efficacy against spike structures with mutations in this region. Indeed, GlycoGrip1.0 demonstrated weaker signal intensity for Alpha variant spike proteins compared to WT spikes, due to key deletions in the Alpha spike NTD relative to WT. In the current work, we return to the cell-surface and investigate the potential of using ACE2, the primary host-cell receptor as an ever-relevant signaling probe: if the virus infects, GlycoGrip2.0 powered by ACE2 will detect.

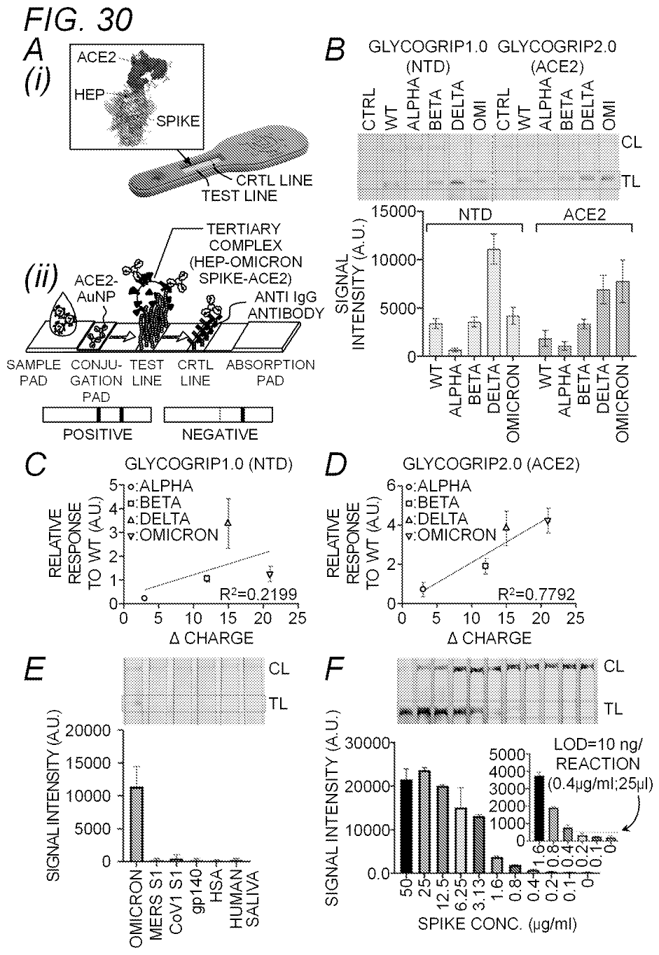

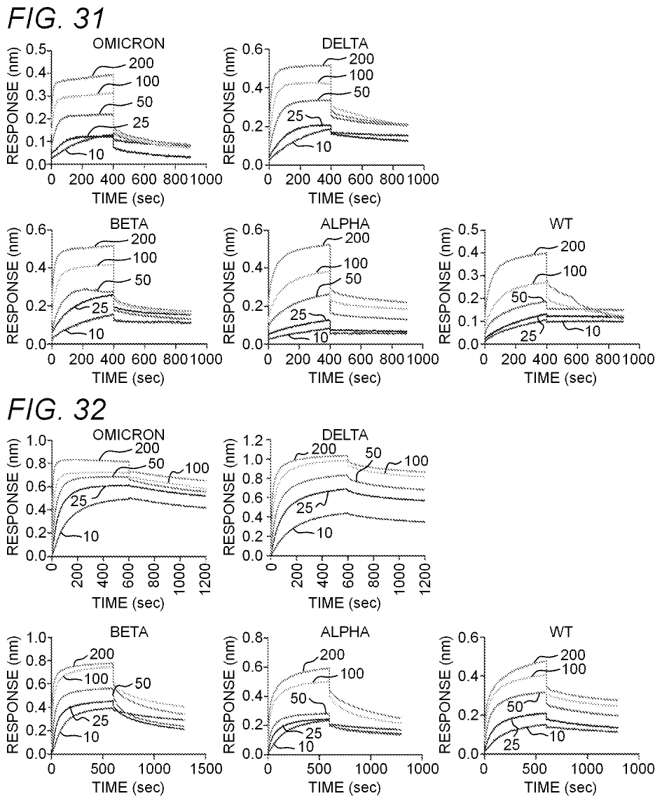

Based on these mechanistic insights, we sought to redesign our GlycoGrip1.0 LFSA device to generate GlycoGrip2.0 to include ACE2 conjugated Au-nanoparticles (AuNP) as spike signaling probes (FIG. 30, Panel A (i, ii)). In GlycoGrip1.0, we used an N-terminal domain binding (NTD) anti-spike antibody (NTD Ab) as the AuNP conjugated signaling probe and demonstrated sensitive and specific detection of SARS-CoV-2 WT, Alpha, Beta, and Delta.²⁴ Given the high degree of spike mutations particularly present in the NTD, GlycoGrip1.0 is potentially vulnerable to decreasing signal intensity if mutations in this region became too extensive. Therefore, we hypothesized that harnessing HEP and ACE2 could overcome this challenge while yielding enhanced sensitivity by exploiting the synergistic complexation of HEP/Spike/ACE2.

For both generations of GlycoGrip generations, when a sample contains spike protein, a double banded signal will appear on the lateral flow strip: one band at the test line (TL) band indicating ternary complex formation between HEP, spike, and the signaling probe, and one band at the control line (CL) indicating binary complex formation between the signaling probe and an anti-signaling probe antibody. We compared GlycoGrip1.0 and GlycoGrip2.0 against all VOC spike proteins (FIG. 30, Panel B). We observed that, while GlycoGrip1.0 still signaled well for Omicron spike, signal intensity dropped significantly relative to Delta. However, the trend observed for WT, Alpha, Beta, and Delta spikes on GlycoGrip1.0 was similar to that reported previously.⁸⁹ Notably, GlycoGrip2.0 elicited the strongest signal for Omicron spikes compared to GlycoGrip1.0. Moreover, we observed a clear trend: signal intensity on our GlycoGrip2.0 increases along with the variant timeline (FIG. 30, Panel B) in a manner strikingly similar to the increase in total spike charge. Plotting this change in spike charge (i.e., total charge changes relative to WT spike) against relative signal intensity on our reconfigured GlycoGrip2.0, we see these two quantities correlate with one another: R²=0.7792 (FIG. 30, Panel E) while there was no clear trend for GlycoGrip1.0 (FIG. 30, Panel C): R²=0.2199. This correlation is striking given that, with HEP as the capture agent and ACE2 as the signaling probe, GlycoGrip2.0 can be seen as a simplified model for the cell surface environment. These results indicate that, in contrast to antibody-based detection, our cell-surface mimetic sensor easily and effectively adapts to viral mutations, suggesting a novel paradigm shift in designing LFSA platforms to sensing viral antigens with high mutation rates.

Finally, we investigated the selectivity and sensitivity of our GlycoGrip2.0 specifically for detection of Omicron spike proteins. To determine the selectivity, we interrogated our sensor with related coronavirus (MERS, CoV1) and HIV (gp140) envelope proteins along with relevant complex proteins such as human serum albumin (HSA). To illustrate GlycoGrip's feasibility when used against complex biologically relevant media, we also tested our sensor against a non-infected human saliva sample to check the false-positive signal from the complex biological samples. As shown in FIG. 30, Panel E, GlycoGrip2.0 selectively captures and signals for Omicron spike proteins while not binding to related viral proteins, HSA, or other saliva matrix elements. Finally, Omicron was detectable as low as 40 ng/reaction (1.6 μg/ml, 25 μl) with ACE2, and 78 ng/reaction with NTD Abs (data not shown). We then adopted a silver staining method to further enhance detection 4-fold for Omicron spikes: down to 10 ng/reaction (0.4 μg/ml, FIG. 30, Panel F).

In all, these results indicate GlycoGrip is selective for SARS-CoV-2 spike proteins, signals strongly in the presence of Omicron spikes, and is rapidly adaptable and deployable within the context of the ever-evolving COVID-19 public health crisis. Furthermore, as SARS-CoV-2 continues to adapt to niche evolutionary pressures within glycocalyx, GlycoGrip's detection capacities will likely maintain and even strengthen, co-evolving with the spike protein sequence.

As the COVID-19 pandemic now progresses into its third year, public health experts continue to scan the epidemic-horizon for new variants. Delineating the environmental and immunological pressures driving SARS-CoV-2 genomic adaptation can help predict the likely range of future mutations, and the potential impacts of those mutations on infection, re-infection, hospitalization, and mortality rates. In this work, we revealed that increased total charge on the spike proteins of SARS-CoV-2 variants, due to the progression of positively charged mutations, strengthens long-range electrostatic interactions with the negatively-charged host cell surface. Furthermore, we showed that redistribution of positive and negative charges on evolving spike protein variants, particularly for Omicron, which adopts a striking ‘bulls-eye’ like patch of positive charge near the RBM, selectively enhances the rate and strength of HS binding to exposed RBMs. In this way, Omicron SARS-CoV-2 kinetically increases the local concentration of ACE2 binding-ready spikes at the cell surface and unlocks a key synergy between HS and ACE2. We believe this remapping of positive charge on the SARS-CoV-2 spike protein is an evolutionary driver for the optimization of electrostatic interactions of spike proteins with both HS and ACE2, thereby increasing the rate of viral entry. With these conclusions in hand, one could predict that emerging variants will exhibit additional charge redistribution to further fine-tune these interactions and in-turn increase SARS-CoV-2 infectivity.

Finally, we leveraged our findings of a favorable tertiary complex formation between the Omicron spike protein, HS, and ACE2 to develop the GlycoGrip2.0 sensor, and demonstrated its ability to “co-evolve” alongside the SARS-CoV-2 genome, and improve its detection of all variants of concern. By harnessing the primary (ACE2) and secondary (HS) cellular receptors in one sensor, GlycoGrip2.0 essentially serves as a minimal model of the glycocalyx environment, which may also serve as a useful platform for viral surveillance. This highlights the advantage of glycocalyx inspired sensing in a rapidly adapting public health crisis, as it is quickly reconfigurable and employable against evolving variants. As the COVID-19 pandemic is still ongoing, due to continuous evolution of the virus, we believe glycocalyx-inspired LFSAs will be a great benefit for global health monitoring power, not only for SARS-CoV-2 but other rapidly mutating respiratory viral antigens.

### Tables

TABLE 2
Complete list of all mutations per variant considered for modeling and
charge calculations in this work. Glycan contribution calculated according to
Watanabe et al.⁶ and with glycans chosen consistent to Casalino et al.⁵
with 14 sialic acids included (total glycan charge of −14).

| | Mutations from WT/2019 (charge change relative to | | |
|---|---|---|---|
| Variant | WT due to mutation) | ΔT_{Q} | T_{Q} |
| WT | | | −11 |
| Alpha | ΔH69-V70 (0), ΔY144 (0), A570D (−1), D614G (+1), | (+1)*3 = +3 | −8 |
| | P681H (0), T716I (0), S982A (0), D1118H (+1) | | |
| Beta | D80A (+1), D215G (+1), ΔL241-L242-A243 (0), K417N | (+4)*3 = +12 | +1 |
| | (−1), E484K (+2), N501Y (0), D614G (+1), A701V (0) | | |
| Delta | T19R (+1), G142D (−1), ΔE156−F157 (+1), R158G (−1), | (+5)*3 = +15 | +4 |
| | L452R (+1), T478K (+1), D614G (+1), P681R (+1), | | |
| | D950N (+1), ΔN17-Glycan (0) | | |
| Omicron | A67V (0), ΔH69-V70 (0), T95I (0), G142D (−1), | (+7)*3 = +21 | +10 |
| (BA.1) | ΔV143-Y144-Y145 (0), ΔN211 (0), L212I, ins214EPE | | |
| | (−2), G338D (−1), S371L (0), S373P (0), S375F (0), | | |
| | K417N (−1), N440K (+1), G446S (0), S466N (0), T478K | | |
| | (+1), E484A (+1), Q493R (+1), G498R (+1), N501Y (0), | | |
| | Y505H (0), T547K (+1), D614G (+1), H655Y (0), N679K | | |
| | (+1), P681H (0), N764K (+1), D796Y (+1), N856K (+1), | | |
| | Q954H (0), N969K (+1), L981F (0) | | |

TABLE 3
Complete list of all titratable residues and their selected protonation states each
spike/ACE2 structure to pH = 7.4, as calculated by PROPKA. Full pKa calculation
data can be found in the shared files associated with this supporting information.

| pH | Prot | Cnf | P.S. | Residue IDs |
|---|---|---|---|---|
| 7.4 | WT | Clo. | ASP | 40 53 80 88 111 138 178 198 215 228 253 287 290 294 364 389 |
| | | | | 398 405 420 427 428 442 467 568 571 574 578 586 614 627 663 |
| | | | | 767 745 775 796 808 820 830 839 843 848 867 936 950 979 985 |
| | | | | 994 1041 1084 1118 1127 1139 |
| | | | ASPP | none |
| | | | GLU | 96 132 154 156 169 180 191 224 281 598 309 324 340 406 465 |
| | | | | 471 484 516 554 583 619 654 661 702 725 748 773 780 819 868 |
| | | | | 918 988 990 1017 1031 1072 1092 1111 |
| | | | GLUP | none |
| | | | HSD | 146 207 245 519 1058 |
| | | | HSE | 49 66 69 625 655 1048 1064 1083 1101 |
| | | | HSP | none |
| 7.4 | WT | 1up | ASP | 40 53 80 88 111 138 178 198 215 228 253 287 290 294 364 389 |
| | | | | 398 405 420 427 428 442 467 568 571 574 578 586 614 627 663 |
| | | | | 767 745 775 796 808 820 830 839 843 848 867 936 950 979 985 |
| | | | | 994 1041 1084 1118 1127 1139 |
| | | | ASPP | none |
| | | | GLU | 96 132 154 156 169 180 191 224 281 598 309 324 340 406 465 |
| | | | | 471 484 516 554 583 619 654 661 702 725 748 773 780 819 868 |
| | | | | 918 988 990 1017 1031 1072 1092 1111 |
| | | | GLUP | none |
| | | | HSD | 146 207 245 519 1058 |
| | | | HSE | 49 66 69 625 655 1048 1064 1083 1101 |
| | | | HSP | none |
| 7.4 | Del | Clo. | ASP | 40 53 80 88 111 138 142 178 198 215 228 253 287 290 294 364 |
| | | | | 389 398 405 420 427 428 442 467 568 571 574 578 586 627 663 |
| | | | | 737 745 775 796 808 820 830 839 843 848 867 936 979 985 994 |
| | | | | 1041 1084 1118 1127 1139 |
| | | | ASPP | none |
| | | | GLU | 96 132 154 169 180 191 224 281 298 309 324 340 406 465 471 |
| | | | | 484 516 554 583 619 654 661 702 725 748 773 780 819 868 918 |
| | | | | 988 990 1017 1031 1072 1092 1111 |
| | | | GLUP | none |
| | | | HSD | 519 625 655 1058 1083 1088 |
| | | | HSE | 49 66 69 146 207 245 1048 1064 1101 |
| | | | HSP | None |
| 7.4 | Del | 1up | ASP | 40 53 80 88 111 138 142 178 198 215 228 253 287 290 294 364 |
| | | | | 389 398 405 420 427 428 442 467 568 571 574 578 586 627 663 |
| | | | | 737 745 775 796 808 820 830 839 843 848 867 936 979 985 994 |
| | | | | 1041 1084 1118 1127 1139 |
| | | | ASPP | none |
| | | | GLU | 96 132 154 169 180 191 224 281 298 309 324 340 406 465 471 |
| | | | | 484 516 554 583 619 654 661 702 725 748 773 780 819 868 918 |
| | | | | 988 990 1017 1031 1072 1092 1111 |
| | | | GLUP | none |
| | | | HSD | 519 625 655 1058 1083 1088 |
| | | | HSE | 49 66 69 146 207 245 1048 1064 1101 |
| | | | HSP | None |
| 7.4 | Omi | Clo. | ASP | 40 53 80 88 111 138 142 178 198 215 228 253 287 290 294 339 |
| | | | | 364 389 398 405 420 427 428 442 467 568 571 574 578 586 627 |
| | | | | 663 737 745 775 808 820 830 839 843 848 867 936 950 979 985 |
| | | | | 994 1041 1084 1118 1127 1139 |
| | | | ASPP | none |
| | | | GLU | 96 132 154 156 169 180 191 2141 (inserted E) 2143 (inserted E) |
| | | | | 224 281 298 309 324 340 406 465 471 516 554 583 619 654 661 |
| | | | | 702 725 748 773 780 819 868 918 988 990 1017 1031 1072 1092 |
| | | | | 1111 |
| | | | GLUP | None |
| | | | HSD | 146 207 245 519 681 1058 1088 |
| | | | HSE | 49 66 505 625 954 1048 1064 1083 1101 |
| | | | HSP | none |
| 7.4 | Omi | 1up | ASP | 40 53 80 88 111 138 142 178 198 215 228 253 287 290 294 339 |
| | | | | 364 389 398 405 420 427 428 442 467 568 571 574 578 586 627 |
| | | | | 663 737 745 775 808 820 830 839 843 848 867 936 950 979 985 |
| | | | | 994 1041 1084 1118 1127 1139 |
| | | | ASPP | none |
| | | | GLU | 96 132 154 156 169 180 191 2141 (inserted E) 2143 (inserted E) |
| | | | | 224 281 298 309 324 340 406 465 471 516 554 583 619 654 661 |
| | | | | 702 725 748 773 780 819 868 918 988 990 1017 1031 1072 1092 |
| | | | | 1111 |
| | | | GLUP | None |
| | | | HSD | 146 207 245 519 681 1058 1088 |
| | | | HSE | 49 66 505 625 954 1048 1064 1083 1101 |
| | | | HSP | none |
| 7.4 | ACE2 | — | ASP | 30 38 67 111 136 157 198 201 206 213 216 225 269 292 295 299 |
| | | | | 303 335 350 355 367 368 382 427 431 471 494 499 509 543 597 |
| | | | | 609 615 630 637 669 693 713 719 |
| | | | ASPP | none |
| | | | GLU | 22 23 35 37 56 57 75 87 110 140 145 150 160 166 171 181 182 |
| | | | | 189 197 208 224 227 231 232 238 310 312 329 375 398 402 406 |
| | | | | 430 433 435 457 467 479 483 489 495 527 536 549 564 571 589 |
| | | | | 634 639 667 668 699 701 723 |
| | | | GLUP | none |
| | | | HSD | 34 195 239 373 374 378 417 493 |
| | | | HSE | 228 241 265 345 401 505 535 540 |
| | | | HSP | none |

Prot. = protein. Cnf = conformational state, relevant to spike protein structures only. P.S. = protonation state. Clo. = spike in closed conformational state. 1up = spike in 1up/open conformational state. WT = Wild Type. Del. = Delta. Omi. = Omicron. ACE2 = angiotensin converting enzyme 2. ASP = deprotonated/negatively charged aspartate. ASPP = protonated/neutral aspartate. GLU = deprotonated/negatively charged glutamate. GLUP = protonated/neutral glutamate. HSD = singly protonated/neutral histidine with protonation on the N_{d} atom. HSE = singly protonated/neutral histidine with protonation on the Nₑ atom. HSP = double protonated/positively charged histidine with protonation on N_{d} and Ne atoms.

TABLE 4
Complete list of protein residues per GAG binding hotspot as predicted
by ensemble-based docking with AutoDock and Kmeans clustering.

| | Protein Residues: Residue Name, Residue Number, Chain ID |
|---|---|
| Site | Glycans: Glycan Protein Residue Name, Protein Residue Number, (Protein Chain ID) |
| A | Arg403A, Asp405A, Glu406A, Asn417A, Lys417A, Tyr421A, Ser443A, Lys444A, |
| | Val445A, Gly446A, Asn448A, Tyr449A, Tyr451A, Leu452A, Tyr453A, Leu455A, |
| | Phe456A, Lys458A, Ser459A, Asn460A, Lys462A, Ser477A, Phe490A, Leu492A, |
| | Gln493A, Arg493A, Ser494A, Tyr495A, Ser496A, Phe497A, Gly496A, Gln498A, |
| | Arg498A, Pro499A, Thr500A, Asn501A, Tyr501A, Gly502A, Val503A, Gly504A, |
| | Tyr505A, His505A, Gln506A, Pro507A, Phe342B, Asn343B, Tyr369B, Asn370B, |
| | Leu371B, Ser371B, Ala372B, Ser373B, Pro373B, Ser375B, Phe375B, Phe374B, |
| | Trp436B, Asn437B, Ser438B, Asn439B, Asn440B, Lys440B, Leu441B, Gly447B, |
| | Glycan N343(B) |
| B | Ser49B, Arg346B Phe347B, Ala348B, Val350B, Tyr351B, Ala352B, Trp353B, |
| | Leu371C, Ala372C, Phe374C, Phe400B, Val401B, Ile402B, Arg403B, Glu406B, |
| | Gly416B, Lys417B, Asn417B, Ile418B, Ala419B, Tyr421B, Asn422B, Tyr423B, |
| | Leu441B, Asp442B, Ser443B, Lys444B, Gly447B, Asn448B, Tyr449B, Asn450B, |
| | Tyr451B, Leu452B, Arg452B, Tyr453B, Arg454B, Leu455B, Phe456B, Arg457B, |
| | Leu461B, Thr470B, Glu471B, Tyr473B, Tyr489B, Phe490B, Pro491B, Leu492B, |
| | Gln493B, Arg493B, Ser494B, Tyr495B, Gly496B, Ser496B, Phe497B, Gln498B, |
| | Asn501B, Tyr501B, Tyr505B, His505B, Pro507B, Glycan N165(C), Glycan N343(C) |
| C | Asn370A, Ser371A, Ser373A, Phe377A, Lys378A, Cys379A, Tyr380A, Gly381A, |
| | Val382A, Ser383A, Pro384A, Thr385A, Lys386A, Ala411A, Pro412A, Gln414A, |
| | Asp427A, Asp428A, Phe429A, Thr430A, Gly431A, Leu517A, Tyr51C, Ser349C, |
| | Val350C, Ala352C, Ile402C, Arg403C, Glu406C, Gly416C, Asn417C, Lys417C, |
| | Ile418C, Ala419C, Asp420C, Tyr421C, Asn422C, Tyr423C, Tyr449C, Asn450C, |
| | Tyr451C, Leu452C, Arg452C, Tyr453C, Arg454C, Leu455C, Phe456C, Lys458C, |
| | Ser459C, Leu461C, Asp467C, Ile468C, Arg469C, Thr470C, Glu471C, Ile472C, |
| | Tyr473C, Gln474C, Ala475C, Glu484C, Cys488C, Tyr489C, Phe490C, Pro491C, |
| | Leu492C, Gln493C, Arg493C, Ser494C, Tyr495C, Gly496C, Ser496C, Phe497C, |
| | Tyr501C, Glycan N165(A), Glycan N343(A) |
| D | Ser13C, Cys15C, Val16C, Leu18C, Ala67C, Val67C, Ile68C, His69C, Ser71C, |
| | Phe79C, Arg78C, Asn81C, Glu96C, Lys97C, Ser98C, Asn99C, Ile100C, Ile101C, |
| | Arg102C, Gly103C, Leu118C, Val120C, Asn121C, Asn122C, Pro139C, Ala123C, |
| | Thr124C, Asn125C, Val127C, Asp138C, Pro139C, Phe140C, Leu141C, Asp142C, |
| | Gly142C, Val143C, Tyr144C, Tyr145C, His146C, Lys147C, Asn148C, Asn149C, |
| | Lys150C, Ser151C, Trp152C, Met153C, Glu154C, Gly156C, Ser155C, Glu156C, |
| | Phe157C, Arg158C, Val159C, Phe175C, Leu176C, Met177C, Asp178C, Leu179C, |
| | Glu180C, Gly181C, Lys182C, Gln183C, Arg190C, Thr240C, Leu241C, Leu242C, |
| | Ala243C, Leu244C, His245C, His245C, Arg246C, Ser247C, Tyr248C, Leu249C, |
| | Thr250C, Ser254C, Gly257C, Trp258C, Thr259C, Ala260C, Gly261C, Ala262C, |
| | Ala263C, Glycan N122(C), Glycan N149(C) |
| E | Asp420A, Tyr421A, Phe456A, Arg457A, Lys458A, Ser459A, Asn460A, Thr385B, |
| | Lys386B, Asn388B, Asp389B, Pro527B, Lys528B, Thr415A, Gly416A, Tyr473A, |
| | Asp364B, Ser366B, Gly526B, Lys529B, Tyr369B, Asn370B, Val367B, Leu455A, |
| | Leu461A, Leu387B, Tyr453A, Arg454A, Pro384B, Lys417A, Ile418A, Tyr489A, |
| | Asp985C, Tyr365B, Lys462A, Val327B, Lys424A, Pro463A, Leu371B, Glycan |
| | N234(B), Glycan N343(B), Glycan T323(B) |
| F | Pro330B, Ile332B, Arg357B, Ile358B, Ser359B, Asn360B, Cys361B, Phe392B, |
| | Thr393B, Asn394B, Val395B, Tyr396B, Glu516B, Leu518B, His519B, Ala520B, |
| | Pro521B, Ala522B, Thr523B, Val524B, Asn544B, Leu560B, Pro561B, Phe562B, |
| | Gln563B, Gln564B, Arg577B, Leu582B, Lys41C, Ile128C, Phe168C, Glu169C, |
| | Tyr170C, Val171C, Ser172C, Gln173C, Pro174C, Phe175C, Tyr200C, Lys202C, |
| | Pro225C, Leu226C, Val227C, Asp228C, Leu229C, Pro230C, Glycan N331(B), |
| | Glycan N122(C) |
| G | Ile119A, Thr124A, Asn125A, Val126A, Val127A, Ile128A, Phe168A, Glu169A, |
| | Tyr170A, Val171A, Ser172A, Gln173A, Pro174A, Phe175A, Leu179A, Ile203A, |
| | Leu226A, Val227A, Asp228A, Leu229A, Pro230A, Arg357C, Ser359C, Asn360C, |
| | Asn394C, Tyr396C, Thr523C, Glycan N122(A), Glycan N149(A), Glycan N331(C), |
| H | Arg357A, Ser359A, Asn360A, Thr393A, Asn394A, Ala520A, Pro521A, Ala522A, |
| | Thr523A, Pro561A, Phe562A, Lys41B, Leu117B, Ile119B, Val120B, Asn121B, |
| | Thr124B, Asn125B, Val126B, Val127B, Ile128B, Lys129B, Val130B, Phe168B, |
| | Glu169B, Tyr170B, Val171B, Ser172B, Gln173B, Pro174B, Phe175B, Leu176B, |
| | Met177B, Leu179B, Phe192B, Tyr200B, Phe201B, Lys202B, Ile203B, Tyr204B, |
| | Ser205B, Glu224B, Pro225B, Leu226B, Val227B, Asp228B, Leu229B, Pro230B, |
| | Ile231B, Gly232B, Glycan N331(A), Glycan N122(B), Glycan N282(B) |
| I | Ser325A, Val327A, Val382A, Ser383A, Thr385A, Lys386A, Leu387A, Asn388A, |
| | Asp389A, Leu390A, Leu518A, Lys528A, Asn540A, Phe541A, Asn542A, Gly545A, |
| | Leu546A, Thr547A, Gly548A, Glu748B, Asn978B, Ile980B, Leu981B, Ser982B, |
| | Arg983B, Leu984B, Asp985B, Lys986B, Ala989B, Thr747B, Asp979B, Ser746B, |
| | Phe329A, Leu977B, Phe543A, Asn544A, Thr549A, Val976B, Ile326A, Val987B, |
| | Glu988B, Ile993B, Pro322A, Glu324A, Asp745B, Lys417C, Gln321A, Thr323A, |
| | Val539A, Lys547A, Phe981B, Arg328A, Gly550A, Glucan T323(A), Glycan |
| | S325(A), Glycan N234(A) |
| J | Gln52B, Thr274B, Gln271B, Arg273B, Asp290B, Cys291B, Ala292B, Leu293B, |
| | Glu298B, Ser316B, Asn317B, Phe318B, Arg319B, Val320B, Gln321B, Pro322B, |
| | Thr323B, Glu324B, Lys537B, Cys538B, Asn540B, Thr549B, Val551B, Cys590B, |
| | Ser591B, Phe592B, Gly593B, Val595B, Tyr612B, Glu619B, Val620B, Pro621B |
| | Val622B, Ala623B, Ile624B, His625B, Ala626B, Asp627B, Gln628B, Leu629B, |
| | Thr630B, Pro631B, Thr632B, Trp633B, Arg634B, Val635B, Met740C, Asp745C, |
| | Thr747C, Glycan N234(B), Glycan T323(B) |
| K | Asp737A, Cys738A, Thr739A, Met740A, Ile742A, Cys743A, Gly744A, Asp745A, |
| | Ser746A, Thr747C, Glu748A, Cys749A, Ser750A, Asn751A, Leu752A, Leu753A, |
| | Leu754A, Gln755A, Tyr756A, Phe759A, Gly757A, Ser758A, Cys760A, Thr761A, |
| | Lys764A, Ile993A, Ile997A, Leu1001A, Ser50C, Gln52C, Asp53C, Pro272C, |
| | Thr274C, Glu298C, Lys304C, Ser316C, Asn317C, Phe318C, Arg319C, Thr302C, |
| | Phe592C, Thr630C, Glycan N234(C) |
| L | Leu24B, Pro25B, Pro26B, Ala27B, Tyr28B, Thr29B, Asn30B, Asn61B, Val62B, |
| | Thr63B, Trp64B, Phe65B, His66B, Ile68B, Val70B, Arg78B, Val213B, Arg214B, |
| | Asp215B, Tyr266B, Leu212B, ins-Glu2141B, ins-Pro2142B, ins-Glu2143B, Glycan |
| | N61(B), Glycan N74(B), Glycan N603(B) |
| M | Val47B, Leu48B, His49B, Ser50B, Thr51B, Gln52B, Thr274B, Leu276B, Val289B, |
| | Cys291B, Pro295B, Ser297B, Glu298B, Thr299B, Lys300B, Cys301B, Thr302B, |
| | Leu303B, Lys304B, Ser305B, Phe306B, Thr307B, Val308B, Tyr313B, Gln314B, |
| | Thr315B, Ser316B, Asn317B, Phe318B, Ile569A, Trp633B, Arg634B, Cys738C, |
| | Thr739C, Leu753C, Leu754C, Tyr756C, Gly757C, Ser758C, Phe759C, Cys760C, |
| | Thr761C, Gln762C, Leu763C, Lys764C, Asn764C, Arg765C, Gln957B, Asn960B, |
| | Thr961B, Lys964B, Gln965B, Ser968B, Ser967B, |
| N | Leu48A, His49A, Ser50A, Thr51A, Thr274A, Phe275A, Leu276A, Leu277A, |
| | Val289A, Cys291A, Ser297A, Glu298A, Thr299A, Lys300A, Cys301A, Thr302A, |
| | Leu303A, Lys304A, Ser305A, Phe306A, Thr315A, Thr961A, Lys964A, Leu754B, |
| | Gly757B, Ser758B, Cys760B, Thr761B, Asn764B, Gln52A, Leu296A, Ser316A, |
| | Arg765B, Asp290A, Ala292A, Phe759B, Gln762B, Thr739B, Leu753B, Val308A, |
| | Asp294A, Pro295A, Gln314A, Thr307A, Gln957A, Gln755B, Tyr756B, Tyr313A, |
| | Val597A, Gln965A, Cys738B, Lys764B, Asn317A, Phe318A, Ile569C Leu849A, |
| | Glycan N234(A), Glycan T323(A) |
| O | Ile834A, Ile312C, Val595C, Ser596C, Ile598C, Val608C, Ala609C, Val610C, |
| | Leu611C, Tyr612C, Gln613C, Gly614C, Val615C, Asn616C, Cys617C, Thr618C, |
| | Glu619C, Val620C, Pro621C, Val622C, Ala623C, Ile624C, Leu629C, Thr630C, |
| | Pro631C, Thr632C, Trp633C, Arg634C, Val635C, Tyr636C, Ser637C, Thr638C, |
| | Gly639C, Ser640C, Asn641C, Val642C, Phe643C, Gln644C, Thr645C, Arg646C, |
| | Ala647C, Gly648C, Cys649C, Leu650C, Ile651C, Gly652C, Ala653, Glu654C, |
| | His655C, Tyr655C, Val656C, Ile666C, Ile670C, Cys671C, Ala672C, Gln675C, |
| | Arg682C, Arg683C, Ser686C, Ala688C, Ser689C, Gln690C, Ser691C, Ile692C, |
| | Ile693C, Ala694C, Tyr695C, Glycan N616(C), Glycan N657(C) |
| P | Asn616B, Gln644B, Thr645B, Arg646B, Ala647B, Gly648B, Ala668B, Gly669B, |
| | Ile670B, Pro812C, Ser813C, Lys814C, Arg815C, Phe823C, Leu828C, Ala829C, |
| | Asp830C, Ala831C, Gly832C, Phe833C, Ile834C, Lys835C, Gln836C, Tyr837C, |
| | Pro862C, Pro863C, Leu865C, Thr866C, Asp867C, Glu868C, Met869C, His1058C, |
| | Glycan N616(B), Glycan N657(B), Glycan N282(C) |
| Q | Ala609A, Val610A, Leu611A, Tyr612A, Val615A, Asn616A, Cys617A, Thr618A, |
| | Glu619A, Val620A, Pro621A, Leu629A, Thr632A, Gly639A, Ser640A, Asn641A, |
| | Val642A, Phe643A, Gln644A, Thr645A, Gly648A, Cys649A, Leu650A, Ile651A, |
| | Gly652A, Ala653A, Glu654A, His655A, Val656A, Ile670A, Arg682A, Ala694A, |
| | Thr630A, Arg646A, Gln628A, Ile666A, His655A, Arg634A, Asn657A, Arg681A, |
| | Arg683A, Ile693A, Tyr695A, Thr638A, His625A, Thr696A, Ala623A, Ser686A, |
| | Ser691A, Ile692A, Tyr655A, Glycan N616(A), Glycan N657(A) |
| R | Leu1024A, Thr1027A, Lys1028A, Glu1031A, Ser1037A, Lys1038A, Arg1039A, |
| | Val1040A, Asp1041A, Phe1042A, Cys1043A, Glu780B, Val781B, Gln784B, |
| | Ala1020B, Ser1021B, Ala1022B, Asn1023B, Leu1024B, Ala1025B, Ala1026B, |
| | Thr1027B, Lys1028B, Met1029B, Ser1030B, Glu1031B, Cys1032B, Val1033B, |
| | Leu1034B, Ser1037B, Arg1039B, Phe1042B, Thr1027C, Glu1031C, Arg1039C, |
| | Cys1032A, Leu727B, Cys1043B, Gly1035B, Phe1062B, Trp886B, Asn1023C, |
| | Ser1030C, Gln1036B, Lys1038B, His1064B, Phe1042C, Gly1044A, Lys1045A, |
| | Glu725A |
| S | Val722C, Gly799C, Phe800C, Asn801C, Phe802C, Ser803C, Gln804C, Ile805C, |
| | Gln920C, Lys921C, Leu922C, Ile923C, Ala924C, Asn925C, Gln926C, Phe927C, |
| | Asn928C, Ser929C, Ala930C, Ile931C, Gly932C, Lys933C, Ile934C, Gln935C, |
| | Asp936C, Glycan N709 (B), Glycan N717(C), Glycan N801(C) |

TABLE 5
Fraction of each group measured by mass photometer for Omicron
ternary complex with or without heparan sulfate.

Group

| | A | B | C |
|---|---|---|---|
| Sample | (fraction) | (fraction) | (fraction) |
| Omicron + ACE2 | 0.448 | 0.215 | 0.337 |
| Omicron + ACE2 | 0.453 | 0.240 | 0.307 |
| Omicron + ACE2 | 0.428 | 0.236 | 0.336 |
| Average (Standard | 0.443 | 0.230 | 0.327 |
| Deviation) | (0.014) | (0.013) | (0.017) |
| Omicron + HS + ACE2 | 0.468 | 0.395 | 0.137 |
| Omicron + HS + ACE2 | 0.349 | 0.469 | 0.181 |
| Omicron + HS + ACE2 | 0.316 | 0.488 | 0.195 |
| Average (Standard | 0.378 | 0.451 | 0.171 |
| Deviation) | (0.080) | (0.050) | (0.030) |

TABLE 6
Fraction of each group measured by mass photometer for
Delta ternary complex with or without heparan sulfate.

| | Group | | |
|---|---|---|---|
| | A | B | C |
| Sample | (fraction) | (fraction) | (fraction) |
| Delta + ACE2 | 0.447 | 0.147 | 0.406 |
| Delta + ACE2 | 0.438 | 0.152 | 0.409 |
| Delta + ACE2 | 0.421 | 0.174 | 0.405 |
| Average (Standard | 0.435 (0.013) | 0.158 (0.014) | 0.407 (0.002) |
| Deviation) | | | |
| Delta + HS + ACE2 | 0.507 | 0.145 | 0.348 |
| Delta + HS + ACE2 | 0.473 | 0.155 | 0.372 |
| Delta + HS + ACE2 | 0.545 | 0.191 | 0.264 |
| Average (Standard | 0.508 (0.036) | 0.163 (0.024) | 0.328 (0.057) |
| Deviation) | | | |

TABLE 7
Fraction of each group measured by mass photometer for
WT ternary complex with or without heparan sulfate.

| | Group | | |
|---|---|---|---|
| | A | B | C |
| Sample | (fraction) | (fraction) | (fraction) |
| WT + HS + ACE2 | 0.573 | 0.312 | 0.115 |
| WT + HS + ACE2 | 0.697 | 0.224 | 0.079 |
| WT + HS + ACE2 | 0.597 | 0.254 | 0.150 |
| Average (Standard | 0.622 (0.066) | 0.263 (0.045) | 0.115 (0.035) |
| Deviation) | | | |
| WT + HS + ACE2 | 0.517 | 0.336 | 0.147 |
| WT + HS + ACE2 | 0.641 | 0.209 | 0.150 |
| WT + HS + ACE2 | 0.553 | 0.276 | 0.171 |
| Average (Standard | 0.570 (0.064) | 0.274 (0.064) | 0.156 (0.013) |
| Deviation) | | | |

### Materials and Methods

### Computational Methods

### WT, Delta, Omicron Spike System Construction, MD Simulation, and Clustering

Fully glycosylated, all-atom models of WT, Delta, and Omicron SARS-CoV-2 spike glycoprotein head domains (residues 13 to 1140) were constructed according to the following protocols.

WT: For construction of our WT “closed”/all RBD down system, a cryo-EM structure with 2.8 A resolution was used (PDB ID 6VXX).⁹³ To improve the accuracy of our model, we incorporated fully resolved NTD, RBD, and pre-fusion loops from another closed spike structure (PDB ID 7JJI).⁹⁴ For construction of our WT “open”/1 RBD up system, a cryo-EM structure with 3.46 A resolution was used (PDB ID 6VSB) (cite). To improve the accuracy of our open model, we incorporated fully resolved RBD in an up state bound to ACE2 (PDB ID 6M17) and fully resolved NTD and pre-fusion loops (PDB ID 7JJI).

Delta: We used the WT open and closed structures as described above as the basis for construction of our Delta variant closed and open spike glycoprotein systems. To account for the mutation profile in the Delta variant, we induced single point mutations using the “mutate” command in psfgen. Experimental data showed significant rearrangement/structural remodeling of the NTD on Delta, so we incorporated a cryo-EM structure of the remodeled Delta variant NTD (PDB ID 7SO9) (cite).

Omicron: For construction of our Omicron variant closed system, a cryo-EM structure with 3.36 A resolution was used (PDB ID 7TF8)⁹⁵ as the base structure. Missing loops from the furin cleavage site in the aforementioned Omicron PDB were grafted in from PDB ID 6VSB. For construction of our Omicron variant open system, a cryo-EM structure with 3.40 A resolution was used (PDB ID 7TEI)⁹⁵ as the base structure. Missing loops in the furin cleavage site, fusion peptide, and RBD were grafted in from PDB ID 6VSB. For both open and closed omicron spike glycoprotein systems, we incorporated a cryo-EM structure of a fully resolved Omicron NTD (PDB ID 7K4N).⁹⁶

Glycosylation/Protonation/Solvation/Neutralization: All spike models were then glycosylated following the same glycoprofile as used by Casalino et al.⁹⁷, consistent with Watanabe et al.⁹⁸ Protonation states were assigned by performing stand-alone PROPKA⁹⁹ so that the glycan atoms were considered in the calculation, however protonation states (HSE vs HSD) for histidines were assigned by use of PROPKA through Schrödinger's Protein Preparation Wizard tool.¹⁰⁰ AutoIMD, a VMD tool,¹⁰¹ was used to resolve any glycan/protein clashes or ring penetrations in our glycoprotein systems. Glycoprotein models were then each solvated in explicit water boxes of 215×215×215 Å⁹⁵ and neutralized with 0.15 M NaCl.

Molecular Dynamics Simulations: All structures (6 models in total) were then subjected to the following Molecular Dynamics (MD) simulation protocol (1 replica each) with NAMD2.14,^{102,103} all atoms described according to CHARMM36m all-atom force field:^{104.105} 20,000 steps of Steepest Descent minimization for TIP3 water molecules and NaCl ions. Protein and glycan atoms were held fixed with a Lagrangian constraint. Heating of the solvated system from 10K to 310K by increments of 25K with protein and glycan atoms held in light restraint according to a force constant of 1 kcal/mol/Å. With each increase in temperature, 10080 steps (1 fs/step) of MD simulation were performed within the NVT ensemble. Once the temperature reached 310K, 0.5 ns of NVT equilibration was performed with restraints maintained. NpT equilibration (310K, 1.01325 bar) for 0.5 ns (2 fs/step) with restraint (force constant=1 kcal/mol/Å) applied to all protein backbone atoms. Pressure was maintained a Langevin barostat. Box cell dimensions were set to flexible during pressure equilibration. NpT free (no restraints) production (310K, 1.01325 bar) simulations for 50 ns (2 fs/step). 50 ns NpT production runs were performed on TACC Frontera. As system pressure was equilibrated in the prior step, box cell flexibility was turned off in this step (useFlexibleCell=no).

Clustering: In preparation for ensemble-based spike/GAG docking studies with AutoDock Vina,^{106,107} we selected the final frame of each 50 ns MD simulation to serve as a rigid-docking receptor. We also clustered the 50 ns trajectories to generate 5 other receptor structures per spike glycoprotein model. The 50 ns trajectories were clustered in Python (with MD Analysis^{108,109} and Scikit-learn¹¹⁰) according to the following protocol: To remove global rotational and translational degrees of freedom before clustering, VMD¹⁰¹ was used to align trajectories according to minimum Root Mean Square Deviation (RMSD) distance of all Cₐ atoms from their first frame positions. Water and ion atoms were stripped from resultant aligned trajectories. Aligned trajectories were then opened as universes in MDAnalysis^{108,109} wherein the RMSDs of Cₐ atoms and glycan carbon atoms were calculated for each frame. Python Scikit-learn's¹¹⁰ Kmeans clustering package was then used to cluster all frames according to Cₐ atom and glycan carbon atom RMSDs, and the knee locator algorithm was used to select the optimal number of clusters per simulation. Representative structures—i.e., those simulation frames closest in RMSD space from the true cluster center—for the 5 most populated clusters were then selected for ensemble-based docking with AutoDock Vina.^{106,107} PSF/PDB pairs were generated for each structure selected herein (i.e., for the final frame and for all clustered frames) and have been made available with our shared data sets on the AmaroLab website (https://amarolab.ucsd.edu/covid19.php).

Ensemble-Based Docking with AutoDock Vina

As described above, we selected 6 total structures per spike conformation (the final frame from 50 ns and 5 representative structures from most populated clusters) to serve as receptors in ensemble based rigid docking studies with AutoDock Vina.^{106,107} Each chosen spike receptor structure (3 variants×2 conformational states×6 selected frames=36 total receptor structures) was subjected to the following protocol. To ensure receptor grids generated with AutoDockTools^{106,107} would be similarly applied to each receptor structure, first all receptor PDB structures were aligned to one another according to the S2 domain Cₐ atoms (protein residues 686 to 1140). After alignment, all receptor structures were converted to .pdbqt filetype with AutoDockTools.¹⁰⁷ Heparin (HEP) dimer and tetramer.pdbqt structures were used in previous work¹¹¹ and thus the same files were used in this work (FIG. 35). Per protein structure, the center of the AutoDock^{106,107} receptor grid was defined as the geometric center of the central helix atoms (protein residues 985 to 1000); this was a choice made to ensure relative consistence of the box center for all structures, regardless of closed/1 up conformation. Grid box size was set to 150×150×150 Å\hat{ }3 for all spike structures, this was chosen to ensure RBDs in the 1 up state would still be encompassed within the resultant grid. All docking input files can be found in the datasets shared on the AmaroLab website (amarolab.ucsd.edu/covid19.php). AutoDock Vina^{106,107} settings were applied as follows: energy_range=30, exhaustiveness=80, num_modes=100, a combination which gave 20 binding modes per docking study. To thoroughly sample binding sites and modes on the spike surface, we conducted 20 runs of each docking procedure. A “docking procedure” being defined as one GAG model (HEP dimer or tetramer) docked into one spike receptor structure (e.g., a “docking procedure” could be described as dimeric HEP docked to WT spike in 1 up state, clustered frame #1). Thus, with 20 replicas per docking procedure and 20 resultant binding modes per procedure, we obtained 400 binding modes per docking procedure. With 72 total docking procedures (2 GAG models, 3 spike variants, 2 spike conformational states, 6 receptor structures per spike variant/conformational state), we obtained 28,800 total binding modes from ensemble-based docking in this work.

We then used Scikit-learn's KMeans¹¹⁰ clustering algorithm to cluster the geometric centers of all 28,800 resultant binding modes, and kneed, an inflection point calculation algorithm, to find the optimal number of clusters. From this clustering, we identified 19 distinctive GAG hotspots. To determine which of these 19 sites were accessible to long-chain HEP or HS, as would be encountered on the cell surface, we scanned all selected receptor structures (3 spike variants×2 spike conformational states×6 selected frames per variant/conformational state) to identify all residues within 10 Å of each hotspot centroid (for a full list of residues per site Table 3). As done in previous work, 111 we then used the Shrake-Rupley algorithm¹¹² to calculate the Accessible Surface Area (ASA) of each of these sites from the ˜2 ms of freely available MD trajectories provided by Casalino et al.⁹⁷ We used these simulations to estimate ASA of defined sites, as opposed to our own 50 ns trajectories, because Casalino et al.'s are much longer, and therefore likely to be more representative of conformational variability, especially with respect to glycan degrees of freedom. We calculated ASA for all sites at probe radii ranging from r=1.4 Å (reflective of water molecule probe), 7.2 Å (reflective of small molecule binding or an antibody hypervariable loop), and 18.6 Å (reflective of a small protein binding partner or antibody's variable fragment domain). We compared ASA results calculated at r=7.2 Å between all sites and saw that in the closed state (FIG. 33), sites K, M, N, and R, were highly buried and likely not accessible to ligand binding. However, from the 1 up state, site M becomes moderately exposed. Upon visualizing these sites on the spike structure with VMD we determined sites K, M, N, and R were indeed buried sites, however further investigation will be necessary to determine if site M does indeed become sufficiently exposed after spikes move into the 1 up conformation. We then conducted further statistical analyses of these sites with MDAnalysis^{108,109} as are described in the Supporting Information Results and Discussion below.

### Schrödinger IFD

In past work,¹¹¹ through ensemble-based docking, we identified 3 sites on the spike surface with high affinity to GAGs which could be important for anchoring the spike to long-chain GAG binding modes within the glycocalyx: the RBD cleft, the RBD patch, and the FCS. Ensemble-based docking studies in this current work (described above) reconfirmed the presence of these sites. Additionally, Brownian Dynamics simulations show the importance of the RBM as a potentially adapting kinetic discriminator for HS within the glycocalyx. While we have already incorporated a degree of protein and glycan motion with our ensemble-based docking studies—by selecting clustered spike structures from 50 ns of MD simulation—local binding site conformations can adapt to ligand binding. To assess the degree to which local rearrangements at key binding sites could contribute to GAG binding and to see how these rearrangements do or don't change with the introduction of spike mutations, we conducted site specific flexible ligand-flexible receptor docking simulations with Schrodinger IFD. For docking into the RBD Cleft, RBD Patch, and FCS sites, the final frame from 50 ns simulations of each variant closed spike structure was taken. Since the spike is a trimeric protein, there exist three RBD Cleft, RBD Patch, and FCS sites on the spike structure. To avoid complications and confounding variability due to glycan positioning during flexible docking simulations, we specifically then selected the specific RBD Cleft, RBD Patch, and FCS sites for docking based on which sites were not occupied by glycans in the final frame. This selection was particularly important as Schrodinger IFD does not handle or treat glycan atoms and therefore would not have been able to appropriately include glycan atoms during these studies. For docking into the RBM, we selected the final frame of 50 ns simulations of each variant 1 up spike structure. No special care was needed for selecting frames without glycans in the case of the RBM as the RBM, when in the up/open state, is practically unreachable by spike glycans.

To prepare all spike structures for docking with Schrödinger IFD,¹¹³ all protein structures were first titrated to pH 7.4 with PROPKA.⁹⁹ Then all glycan atoms were removed from protein structures as Schrodinger does not properly treat glycans. HSD, HSE, and HSP residue names were converted to the Schrodinger compatible names HID, HIE, and HIP. Structures were then converted to .mae format and prepared according to OPLS⁴¹¹⁴ force field using Schrödinger Protein Preparation Wizard¹⁰⁰ according to the following settings: missing hydrogen atoms were added (necessary after glycan deletion), bond orders were assigned, disulfide bonds were added (necessary as Schrödinger cannot also take topology files), hydrogen bonds were optimized with PROPKA⁹⁹ at pH 7.4, and a restrained minimization of hydrogen atoms was performed according to energies and forces described by the OPLS⁴¹¹⁴ force field (again necessary after glycan deletion and addition of missing hydrogen atoms). All prepared protein structures will be shared with this work. Heparin and heparan sulfate tetrameric structures (FIG. 35) were prepared for flexible docking with Schrödinger's LigPrep.¹¹⁵

Binding sites for flexible docking were then defined as the center of mass of the following residues, again with care taken to ensure no site was occupied by a glycan in the selected frame from MD simulations:

### WT:

RBD Cleft: (chain B and residues 346 348 349 351 352 354 355 356 357 450 454 466 467 469 489 472 490) or (chain C and residues 113 114 115 132 165 167)
RBD Patch: (chain B and residues 337 356 357 359 360 393 394 516 520 521 523 561 562 577 579 580 582) or (chain C and residues 41 170 172 173 226 227 228)
FCS: (chain B 675 676 677 678 679 680 681 682 683 684 685)
RBM: (chain A and residues 438 to 508)

### Delta:

RBD Cleft: (chain A and residues 346 348 349 351 352 354 355 356 357 450 454 466 467 469 489 472 490) or (chain B and residues 113 114 115 132 165 167)
RBD Patch: (chain B and residues 337 356 357 359 360 393 394 516 520 521 523 561 562 577 579 580 582) or (chain C and residues 41 170 172 173 226 227 228)
FCS: (chain B 675 676 677 678 679 680 681 682 683 684 685)
RBM: (chain A and residues 438 to 508)

### Omicron:

RBD Cleft: (chain A and residues 346 348 349 351 352 354 355 356 357 450 454 466 467 469 489 472 490) or (chain B and residues 113 114 115 132 165 167)
RBD Patch: (chain B and residues 337 356 357 359 360 393 394 516 520 521 523 561 562 577 579 580 582) or (chain C and residues 41 170 172 173 226 227 228)
FCS: (chain B 675 676 677 678 679 680 681 682 683 684 685)
RBM: (chain A and residues 438 to 508)

From each docking procedure, Glide scores were collected and analyzed holistically as well as individual binding modes were inspected to determine interactions of interest within each binding site.

### Brownian Dynamics Simulations

Following the preparation and docking of glycoproteins and ligands, all structures were submitted to the PDB2PQR program^{116,117} to assign atomic partial charges and radii according to the CHARMM36m forcefield.^{104,105} Protonation states for all systems were assigned using PROPKA⁹⁹ at pH of 7.4. Then the “make_apbs_input” and “run_apbs_input” programs in the Browndye2 package¹¹⁸ were used to prepare input files and run APBS 1.5¹¹⁹⁻¹²⁴ to solve the linear Poisson-Boltzmann equation for the creation of electrostatic potential grids for each molecule. Electrostatics calculations, as well as BD simulations, were performed at a temperature of 298.15K, with a NaCl electrolyte concentration of 10 mM, a solvent dielectric of 78, and a solute interior dielectric of 4, and with a grid spacing of 0.5 Å.

BD simulations to study the association kinetics of bimolecular reactions require definitions of reaction criteria. Following the docking procedure, key interacting residues for each of the sites on the glycoproteins were identified. For each site on each monomer of each glycoprotein, the center of mass of these residues was computed. Separately, the center of mass for each ligand was also computed. The distance between the glycoprotein site center of mass and the ligand center of mass was used as the reaction coordinate, and if this distance ever fell below a defined threshold of 14 Å, a “reaction” was assumed to have occurred.

The Browndye2 package¹¹⁸ as used to prepare and run all BD simulations. Hydrodynamics were enabled. Upon independent investigation, we observed anomalous behavior for these systems when desolvation forces were enabled, most likely due to the high magnitude of molecular charges involved. For this reason, we chose not to enable desolvation forces for these simulations. A total of 24 separate systems were simulated on the TACC Frontera supercomputer. For each system, the BD simulations were spread onto a 56-core node and ran for 24 hours. The total number of BD simulations varied between systems, and anywhere from a few hundred to a hundred thousand separate trajectories were completed per system. Following the simulations, the obtained reaction statistics may be used to estimate kₒₙs for each system. BD simulations were performed on TACC Frontera.

To compute the association rate constants to the b-surface, we use the following equation, which is derived from the Smoluchowski equation:^{125,126}

$$k ( r ) = \frac{D \text{?}}{[ 1 - exp \{ \frac{\text{?}}{\text{?}} \} ] {ε}_{0} {ε}_{r} {k}_{B} T}$$

$$\text{?} \text{indicates text missing or illegible when filed}$$

Where k(r) is the association rate constant to the spherical b-surface of radius r, Qₛ is the charge of the substrate, Q_{c} is the charge of the receptor, D is the radial relative diffusion coefficient of the two molecules, ε₀ is the vacuum permittivity, εᵣ is the dielectric constant of the solvent, kB is the Boltzmann constant, and Tis the system temperature.

### Spike RBD+ACE2 MD Simulations

To investigate the stability of the ACE2/RBD interface over the course of the variant timeline, RBD+ACE2 systems were constructed for WT, Delta, and Omicron variants. RBDs were extracted from our full spike WT, Delta, and Omicron models and then aligned to a 2.90 A cryo-EM structure of the WT ACE2/RBD complex (PDB ID 6M17).¹²⁷ ACE2 and aligned RBD complex were extracted for each variant and full glycosylation profile of ACE2 and RBD were replicated from Barros et al.¹²⁸ PROPKA⁹⁹ was used to ensure all protonation states for ACE2 and the RBDs were still appropriate, and they were. Special attention was paid to ensure the Zn²⁺ atoms from ACE2 were retained in RBD/ACE2 system model building. Additionally, special care was taken to make sure there were no residue clashes along the RBD/ACE2 interface as the Delta and Omicron interfaces were constructed from alignment to the WT RBD structure and not resolved experimentally. All systems were solvated in water boxes of ˜130×140×180 Å\hat{ }3 and ionized with 0.15M NaCl. For each RBD/ACE2 system, we then performed 3 replicas of the following MD simulation protocol with NAMD2.14^{102,103} and CHARMM36m all atom force field: 104,105 10,000 steps of Steepest Descent minimization for all atoms (no restraints nor constraints). Heating of the solvated system from 10K to 310K by increments of 25K with protein and glycan atoms held in light restraint according to a force constant of 1 kcal/mol/Å. With each increase in temperature, 10080 steps (1 fs/step) of MD simulation were performed within the NVT ensemble. Once the temperature reached 310K, 0.5 ns of NVT equilibration was performed with restraints maintained. NpT equilibration (310K, 1.01325 bar) for 0.5 ns (2 fs/step) with restraint (force constant=1 kcal/mol/Å) applied to all protein backbone atoms. Pressure was maintained a Langevin barostat. Box cell dimensions were set to flexible during pressure equilibration. GPU accelerated NpT free (no restraints) production (310K, 1.01325 bar) simulations for 50 ns (2 fs/step) conducted with NAMD3.0.¹⁰³ As system pressure was equilibrated in the prior step, box cell flexibility was turned off in this step (useFlexibleCell=no). GPU accelerated NpT production runs were performed on the Hopper GPU cluster at SDSC TSCC. To prepare for analysis, VMD¹⁰¹ was used to align trajectories according to protein Cₐ atomic positions in the first frame, and water and ion atoms were stripped from trajectories. Trajectories were then ported into MDAnalysis^{108,109} as universes where native contacts analysis was performed.

### Dynamical Electrostatic Potential Map Calculations

To confirm the presence of large, positively charged regions on the spike surface we used a time-averaged implementation of Adaptive Poisson Boltzmann Solver (APBS)¹¹⁹⁻¹²⁴ to calculate the electrostatic potential at equally spaced grid points along the spike surface over our aligned 50 ns classical MD simulation trajectories. We calculated electrostatic potential maps for the WT spike in closed and open states as well as for Delta and Omicron BA. 1 variants full-length spike structures in the closed and open states. All resulting ESP volume (.dx) files for each frame of the 50 ns trajectories (1260 frames per simulation) were averaged using the APBS's dxmath functionality. For each structure and each frame, we calculated electrostatic potential maps using the following options, and example input scripts can be found in the downloadable tar.gz file associated with this supporting information:

```
elec name frame
  mg-auto
  dime 321 321 321
  cglen 400 400 400
  fglen 200 200 200
  cgcent mol 1
  fgcent mol 1
  lpbe
  bcfl sdh
  ion charge 1 conc 0.150 radius 1.36375
  ion charge −1 conc 0.150 radius 2.27
  pdie 4.0
  sdie 78.00
  chgm spl2
  srfm smol
  srad 1.4
  swin 0.3
  sdens 10.0
  temp 298.15
  gamma 0.105
  calcenergy total
  write pot dx frame
end
```

### Experimental Methods

### Materials

Heparin (HEP001) was purchased from Galen laboratory supplies. Heparan sulfate from bovine kidney (H7640), Human serum albumin (A3782), and sucrose (S0389), Silver lactate (359750), Hydroquinone (H9003) were purchased from Sigma-Aldrich. Biotin-PEG3-amine (BG-17) was purchased from G-Biosciences. Tween 20 (J20605-AP) was purchased from Thermo Fisher Scientific. Sodium phosphate monobasic (389872500) and Sodium phosphate dibasic (204851000) were purchased from ACROS Organics. Bovine serum albumin (105033) was purchased from MP biomedicals. Gold nanoparticles 10 nm (15703-20) were purchased from Ted Pella Inc. Human ACE2, Fc Tag (AC2-H5257) was purchased from Acro Biosystems. Rabbit anti-human IgG (31143) and Horseradish peroxidase conjugated goat anti-human IgG with Horseradish peroxidase (A18811) was purchased from Invitrogen. Horseradish peroxidase conjugated anti-His Tag antibody (652504) was purchased from Biolegend. Nitrocellulose membrane (FF120HP), sample pad (Whatman CF4 dipstick pad), and absorbent pad (Whatman standard 17) were purchased from Cytiva. SARS-CoV-2 Wild type Spike (40589-V08H4), Delta (40589-V08H10) Spike, and Omicron (40589-V08H26) Spike, and HIV gp140 envelope protein (11677-V08H) were purchased from Sino biological. SARS-CoV-2 Alpha (B.1.1.7) Spike (10796-CV-100) and Beta (B.1.351) spike (10786-CV-100) were purchased from R&D systems. Streptavidin modified BLI biosensor tips (18-5019) and anti-human IgG Fc Capture (AHC) BLI biosensor tips (18-5060) were purchased from Sartorius. N-Terminal domain binding antibody (LT-2000) was purchased from Leinco Technologies. Human saliva pooled from human donors (991-05-P) was purchased from LEE Biosolutions.

### Biotin Conjugation to Heparin

For BLI, ELISA, and LFSA preparation, biotin modified heparin was prepared. Specifically, 2 mg of biotin-PEG3-amine and 2 mg of heparin was dissolved in the 10 mM sodium phosphate buffer pH 7.4. Followed by the addition of the sodium cyanoborohydride (5 mg), and the solution was incubated 24 hr at 60° C. After the incubation, same amount of sodium cyanoborohydride was added and incubated for another 24 hr. Resulting solution was purified by centrifugation with 3k filter to remove the unreacted biotins. Finally, the solution was lyophilized and stored in −20° C. until further use.

### Immobilization and Binding of Heparin, ACE2 to Variant Spike Proteins

To compare the binding of heparin or ACE2 to variant spike proteins, enzyme-linked immunoassay (ELISA) was utilized. Firstly, streptavidin (200 nM; 50 μL) was added to the Nunc maxisorp flat bottom 96 well plate and incubated overnight at 4° C. The plates were washed with 200 μl of 1×PBST (0.05% tween-20) three times to remove unbound streptavidin. Then the plate was blocked with 100 μl of 2% BSA for 1 hr at room temperature and washed with 1×PBST. Biotinylated heparin (800 nM; 50 μL) was incubated for 1 hr and washed thoroughly to remove unbound heparin. SARS-CoV-2 Spike proteins (WT, Alpha, Beta, Delta, and Omicron; 100 nM; 50 μL) in LFSA buffer were incubated for 1 hr and washed three times with 1×PBST. For the signal generation, anti-his tag-HRP (1 μg/ml; 50 μl) was incubated for 30 min. To remove the unbound HRP, plates were thoroughly washed 5 times with 1×PBST. Finally, 50 μl of TMB substrate (Thermo Fisher Scientific, 34028) was added to each well to develop color. The reaction was stopped by adding 50 μl of stop solution (Thermo Fisher Scientific, N600), and absorbance was measured at 450 nm. In case of ACE2 binding, same procedure was used as heparin binding except that ACE2 functionalized plates were prepared by directly absorbing ACE2 (10 μg/mL; 50 μL) to Nunc maxisorp flat bottom 96 well plates at 4° C. overnight instead of streptavidin functionalization.

### Biolayer Interferometry (BLI)

For measuring the binding affinities of heparin or ACE2 to variants of SARS-CoV-2 Spike, biolayer interferometry (BLI) was used. For heparin binding affinity measurement, streptavidin coated BLI tips were functionalized with biotin-heparin (100 μg/ml, 40 μl) for 180 s. Unbound or loosely bound biotin-heparin was washed for 500 s. Heparin functionalized tips were treated with various concentrations of spike protein (0, 10, 25, 50, 100, 200 nM) for 400 sec and dissociation was measured for 500 s. Binding affinity, Dissociation Constant (K_{D}), was calculated with steady-state analysis using the HT 11.1 software. ACE2 binding affinity was measured with the same procedure except that anti-human IgG Fc capture (AHC) BLI tips were utilized ACE2 (1 μg/ml; 40 μl) was loaded. For all measurements, LFSA buffer (10 mM Sodium phosphate, 0.05% tween-20, pH 7.4) was used.

### Checking Ternary Complex Formation (Spike-HS-ACE2) Using ELISA

To check the synergy of heparin and ACE2 binding in spike protein binding, ELISA was utilized. Firstly, streptavidin (200 nM; 50 μl) was added to the Nunc maxisorp flat bottom 96 well plate and incubated overnight at 4° C. The plates were washed with 1×PBST (0.05% tween-20) three times to remove unbound streptavidin. Then the plate was blocked with 2% BSA (100 μl) for 1 hr at room temperature and washed with 1×PBST. Biotinylated heparin (800 nM; 50 μL) was incubated for 1 hr and washed thoroughly to remove unbound heparin. SARS-CoV-2 Spike proteins (100 nM; 50 μl) in LFSA buffer were incubated for 1 hr and washed three times with 1×PBST to remove unbound spike proteins. ACE2 (1 μg/ml; 50 μl) in LFSA buffer was added and incubated for 1 hr. After incubation, each well was washed three times with 1×PBST. Finally, anti-histag-HRP (1 μg/ml; 50 μl) was incubated for 30 min, and plates were thoroughly washed 5 times with 1×PBST. Finally, 50 μL of TMB substrate (Thermo Fisher Scientific, 34028) was added to each well to develop color. The reaction was stopped by adding 50 μL of stop solution (Thermo Fisher Scientific, N600), and absorbance was measured at 450 nm.

### Checking Ternary Complex Formation (Spike-HS-ACE2) Using Mass Photometer

To measure the synergy effect of heparin and ACE2in spike protein binding, mass photometry (Refeyn One mass photometry) was utilized. Microscope coverslips (CG15KH1, 24×50 mm, Thickness 170±5 μm, Thorlabs) were cleaned by sonication in 2-Propanol (A516-4, Fisher Scientific) followed by DW (10 min for each step). Silicone gasket (3 mm diameter×1 mm depth, Grace Bio-labs) was applied to the cleaned coverslip to form a chamber. After preparing the gasket, 10 μl of LFSA buffer (10 mM sodium phosphate, pH 7.4) without tween-20 was first injected to the chamber to find the focus using the autofocus function called droplet-dilution using Acquire MP Sofware provided by the instrument. Samples (Spike, Spike +ACE2, Spike+HS+ACE2) were prepared by incubating Spike (40 μg/ml; 5 μl), ACE2 (16 μg/ml; 2.5 μl), and HS (2 μg/ml; 2.5 μl) for 30 min at room temperature prior to mass photometer. For each acquisition, 10 μl of mixture of protein solution was introduced to the chamber (total volume: 20 μl) and movies of 60 s duration were recorded. Each sample was measured in new chambers (each chamber was used once). All mass photometer data were analyzed with DiscoverMP (2022 ver. R1). Firstly, using a mass calibration curve, ratiometric data was converted to the mass. Then, to check the fraction of the ternary complexes, count numbers for binding event were obtained for each mass range (denoted as group A, B, and C). Obtained count numbers were used to calculate the relative fraction of each and compare the fraction of the ternary complex with or without HS.

### Preparation of Signaling Probes

To prepare the signaling probes, ACE2 and NTD Ab were conjugated to the gold nanoparticles (AuNP). Firstly, 10 nm AuNP (1 ml) was equilibrated with borate buffer (0.1 M pH 8.0; 100 μl), then ACE2 (0.6 mg/ml; 8.3 μl) or NTD Ab (1 mg/ml; 5 μl) was added to prepare the signaling probes for GlycoGrip2.0 and GlycoGrip1.0, respectively. After incubating the resulting solution for 1 hr at room temperature with continuous rotation, 100 μl of 1% BSA was added and incubated for additional 30 min. To remove the unbound proteins, ACE2 or NTD Ab conjugated AuNPs were centrifugated at 22000 g, 4° C. for 45 min and supernatant was removed. AuNP pellet was resuspended in the 1% BSA (1 ml) solution. This procedure was repeated 3 times. Finally, ACE2 or NTD Ab conjugated AuNPs were resuspended in the LFSA buffer and stored in 4° C. until further use.

### Preparation of GlycoGrip LFSA

For the immobilization of the heparin to the nitrocellulose membrane, heparin was conjugated to the streptavidin as previously reported. Briefly, biotin-heparin (2 mg/ml, 50 μl) were incubated with streptavidin (1 mg/ml, 100 μl) for 1 hr. The mixture solutions were purified by centrifugation with 30k amicon filter to remove excess biotin-heparin. The concentration of heparin was measured using Azure A assay. heparin conjugated with streptavidin (300 μM) and rabbit antihuman IgG (1 mg/mL) were dispensed on the nitrocellulose membrane (FF120HP). Dispensed nitrocellulose membrane was dried at 65° C. for 3 min. After drying, the nitrocellulose membrane was blocked with a blocking buffer (1% BSA, 0.05% Tween 20 in 10 mM PB, pH 7.4). Finally, the sample pad (Whatman CF4 dipstick pad) and the absorbent pad (Whatman standard 17) were assembled onto the nitrocellulose membrane. Assembled strips were stored at room temperature with desiccant before use.

### Comparison of the Variant Detection in GlycoGrip1.0 and GlycoGrip2.0

For the comparison of the SARS-CoV-2 variant detection in GlycoGrip1.0 and GlycoGrip2.0, 25 μl of each of the Spike proteins (25 μg/ml) in LFSA buffer were incubated with 25 μl of signaling probes (i.e., 20 nM of NTD Ab-AuNP for Glycogrip1.0, and 20 nM of ACE2-AuNP for Glycogrip2.0) for 30 min at room temperature. Then, the dipstick method was used to compare the detection. Briefly, the resulting solutions were dispensed in the flat bottom 96 well plate and lateral flow strips were dipped for 10 min. After 10 min, image was taken by smartphone camera and signals were quantitatively analyzed by ImageJ software.

### Evaluation of Selectivity and Sensitivity of GlycoGrip2.0

To evaluate the selectivity of the GlycoGrip2.0, 25 μl of each samples including SARS-CoV-2 Omicron spike (25 μg/ml), CoV1 S1 (25 μg/ml), MERS S1 (25 μg/ml), HIV gp140 (group M, CRF07_BC) envelope protein (25 μg/ml), Human serum albumin (50 mg/ml), and Human saliva (25× diluted from the stock solution) was incubated with 25 μl of signaling probe (ACE2-AuNP; 20 nM) for 30 min. Then, signals were generated with dipstick method and analyzed with ImageJ.

For the sensitivity testing, different concentrations of SARS-CoV-2 Omicron spike (0, 0.4, 0.8, 1.6, 3.13, 6.25, 12.5, 25, 50 μg/ml; 25 μl) were incubated with ACE2-AuNP (20 nM; 25 μl) for 30 min. After 30 min, the same dipstick method was utilized. Signal intensity of the test line were analyzed with ImageJ software and the limit of the detection (LOD) was estimated by blank+3 standard deviations. At least 3 independent tests were performed to calculate the LOD.

### Signal Enhancement Analysis

To enhance the sensitivity of the GlycoGrip2.0, silver enhancement methods¹²⁹ was adopted. Specifically, different concentrations of SARS-CoV-2 Omicron spike (0, 0.1, 0.2, 0.4, 0.8, 1.6, 3.13, 6.25, 12.5, 25, 50 μg/ml; 25 μl) were incubated with ACE2-AuNP (20 nM; 25 μl) for 30 min. After 30 min, each solution was dispensed to 96 well plate and LFSA strips were dipped 10 min. After 10 min, Strips were sequentially washed with LFSA buffer (100 μl) and DW (100 μl) for 3 min. Finally, Strips were dipped into the mixture of 0.3% silver lactate and 3% hydroquinone (100 μl each) for 3 min and enhancement reaction was stopped by washing with DW. Signal intensity of the test line were analyzed with ImageJ software and the limit of the detection (LOD) was estimated by blank+3 standard deviations. At least 3 independent tests were performed to calculate the LOD.

### Summary of AutoDock Vina Results

28,800 GAG binding modes were collected from ensemble-based docking studies as laid out in Scheme S1 below. The centers of mass of each of these resultant binding modes were collected passed through kmeans clustering to identify GAG binding “hotspots” on the spike surface. Kneed elbow locator algorithm was used to determine 19 to be the optimal number of clusters of all 28,800 binding modes. These 19 clusters were then given names A-S and their locations on the spike structure were identified. To determine which of these binding sites were “surface exposed”, I.e. sites accessible to long chain GAGs within the glycocalyx, we calculated the Accessible Surface Area (ASA) for each of these sites using the Shrake-Rupley algorithm¹¹² through VMD tools,¹⁰¹ FIG. 33, with a probe radius of r=7.2 Å from WT trajectories shared for closed and 1 up state spike proteins shared by Casalino et al.⁹⁷ Each site was defined as any residue (from all structures considered, i.e., all conformations of WT, Delta, and Omicron structures) within 10 Å of the centroid of said site as defined by kmeans clustering. A probe radius of 7.2 Å was chosen to be consistent with an approaching GAG fragment. All residue numbers per site can be found in Table 3. From ASA results we see that in the closed spike conformation, sites K, M, N, and R are largely inaccessible to GAG fragments within the glycocalyx. However, in the 1 up spike conformation site M becomes marginally exposed due to the lifting up of the neighboring RBD.

To identify any sites of particular importance on a per spike variant basis we then analyzed the distributions of predicted binding energies within each site as given by AutoDock Vina. Both heparin dimers and heparin tetramers bind with relatively similar predicted energies to all sites on a per variant basis, save for a few instances (data not shown). We also compared the distribution of predicted binding energies at each site as a function of spike conformation, i.e., closed versus 1 up states. Again, very little difference could be seen between closed and 1 up spike structures to suggest heparin dimers or tetramers favor binding to either state (data not shown). Given the similarity in predicted energies, and the broad distribution of predicted energies at each site in all resultant binding modes, we cannot predict, at this time, any significant differences in binding affinity at each site resultant from changes in spike sequence. As such we predict binding affinity differences as observed with BLI between heparin and variant spike structures are likely due to effects only captured by use of long-chain heparins or due to kinetic effects such as rate of encounter complex formation.

### Summary of Schrödinger IFD Results

While we have already incorporated a degree of protein flexibility in our docking studies by conducting extensive ensemble-based sampling (i.e., 3 spike proteins (WT, Delta, Omicron)×2 spike states (closed, 1 up)×6 conformations each=36 total spike receptor structures) we were interested in identifying any potential induced fit effects and how such effects may adapt/change over the variant timeline. As such we conducted flexible ligand-flexible receptor docking studies with Schrödinger IFD on targeted sites within the spike protein using heparin and heparan sulfate tetrameric models. From these results we again see predicted binding energies are broad at each site, and there is virtually no difference in these distributions across the variant timeline despite mutations within each site (data not shown). We predict that this is due to the innate flexibility of GAG molecules like heparin and heparan sulfate. These ligands can adapt to mutations within these binding sites and thus still bind at each site. Thus, we predict differences in binding affinity between heparin/heparan sulfate and spike proteins as seen from BLI and ELISA results are likely due to effects that can only be seen at the long-chain binding mode scale or due to kinetic effects not captured in docking studies.

### REFERENCES

(1) WHO Coronavirus (COVID-19) Dashboard. World Health Organization.
(2) World Health Organization. Tracking SARS-CoV-2 Variants of Concern. https://www.who.int/en/activities/tracking-SARS-CoV-2-variants/(accessed 2021 Dec. 12).
(3) Center for Disease Control and Prevention. SARS-CoV-2 Variant Classifications and Definitions. https://www.cdc.gov/coronavirus/2019-ncov/variants/variant-classifications.html (accessed 2022 Aug. 8).
(4) Evolutionary Insight into the Emergence of SARS-CoV-2 Variants of Concern. Nat Med 2022, 28 (7), 1357-1358. https://doi.org/10.1038/s41591-022-01892-2.
(5) Bashor, L.; Gagne, R. B.; Bosco-Lauth, A. M.; Bowen, R. A.; Stenglein, M.; VandeWoude, S. SARS-CoV-2 Evolution in Animals Suggests Mechanisms for Rapid Variant Selection. Proceedings of the National Academy of Sciences 2021, 118 (44). https://doi.org/10.1073/pnas.2105253118.
(6) Otto, S. P.; Day, T.; Arino, J.; Colijn, C.; Dushoff, J.; Li, M.; Mechai, S.; van Domselaar, G.; Wu, J.; Earn, D. J. D.; Ogden, N. H. The Origins and Potential Future of SARS-CoV-2 Variants of Concern in the Evolving COVID-19 Pandemic. Current Biology 2021, 31 (14), R918-R929. https://doi.org/10.1016/j.cub.2021.06.049.
(7) Maher, M. C.; Bartha, I.; Weaver, S.; di Iulio, J.; Ferri, E.; Soriaga, L.; Lempp, F. A.; Hie, B. L.; Bryson, B.; Berger, B.; Robertson, D. L.; Snell, G.; Corti, D.; Virgin, H. W.; Kosakovsky Pond, S. L.; Telenti, A. Predicting the Mutational Drivers of Future SARS-CoV-2 Variants of Concern. Sci Transl Med 2022, 14 (633). https://doi.org/10.1126/scitranslmed.abk3445.
(8) Harvey, W. T.; Carabelli, A. M.; Jackson, B.; Gupta, R. K.; Thomson, E. C.; Harrison, E. M.; Ludden, C.; Reeve, R.; Rambaut, A.; Peacock, S. J.; Robertson, D. L. SARS-CoV-2 Variants, Spike Mutations and Immune Escape. Nat Rev Microbiol 2021, 19 (7), 409-424. https://doi.org/10.1038/s41579-021-00573-0.
(9) McCallum, M.; Walls, A. C.; Sprouse, K. R.; Bowen, J. E.; Rosen, L.; Dang, H. v; DeMarco, A.; Franko, N.; Tilles, S. W.; Logue, J.; Miranda, M. C.; Ahlrichs, M.; Carter, L.; Snell, G.; Pizzuto, M. S.; Chu, H. Y.; van Voorhis, W. C.; Corti, D.; Veesler, D. Molecular Basis of Immune Evasion by the Delta and Kappa SARS-CoV-2 Variants. Science (1979) 2021, 0 (0), eab18506. https://doi.org/10.1126/science.ab18506.
(10) Public Health England. Public Health England Investigation of Novel SARS-CoV-2 Variant 202012/01: Technical Briefing; 2020.
(11) Zhou, D.; Dejnirattisai, W.; Supasa, P.; Liu, C.; Mentzer, A. J.; Ginn, H. M.; Zhao, Y.; Duyvesteyn, H. M. E.; Tuekprakhon, A.; Nutalai, R.; Wang, B.; Paesen, G. C.; Lopez-Camacho, C.; Slon-Campos, J.; Hallis, B.; Coombes, N.; Bewley, K.; Charlton, S.; Walter, T. S.; Skelly, D.; Lumley, S. F.; Dold, C.; Levin, R.; Dong, T.; Pollard, A. J.; Knight, J. C.; Crook, D.; Lambe, T.; Clutterbuck, E.; Bibi, S.; Flaxman, A.; Bittaye, M.; Belij-Rammerstorfer, S.; Gilbert, S.; James, W.; Carroll, M. W.; Klenerman, P.; Barnes, E.; Dunachie, S. J.; Fry, E. E.; Mongkolsapaya, J.; Ren, J.; Stuart, D. I.; Screaton, G. R. Evidence of Escape of SARS-CoV-2 Variant B.1.351 from Natural and Vaccine-Induced Sera. Cell 2021, 184 (9), 2348-2361.e6. https://doi.org/10.1016/j.cell.2021.02.037.
(12) Quandt, J.; Muik, A.; Salisch, N.; Lui, B. G.; Lutz, S.; Krüger, K.; Wallisch, A.-K.; Adams-Quack, P.; Bacher, M.; Finlayson, A.; Ozhelvaci, O.; Vogler, I.; Grikscheit, K.; Hoehl, S.; Goetsch, U.; Ciesek, S.; Türeci, Ö.; Sahin, U. Omicron BA.1 Breakthrough Infection Drives Cross-Variant Neutralization and Memory B Cell Formation against Conserved Epitopes. Sci Immunol 2022. https://doi.org/10.1126/sciimmunol.abq2427.
(13) Shrestha, L. B.; Foster, C.; Rawlinson, W.; Tedla, N.; Bull, R. A. Evolution of the SARS-CoV-2 Omicron Variants BA.1 to BA.5: Implications for Immune Escape and Transmission. Rev Med Virol 2022. https://doi.org/10.1002/rmv.2381.
(14) Mohapatra, R. K.; Kandi, V.; Verma, S.; Dhama, K. Challenges of the Omicron (B.1.1.529) Variant and Its Lineages: A Global Perspective. ChemBioChem 2022, 23 (9). https://doi.org/10.1002/cbic.202200059.
(15) Shao, W.; Zhang, W.; Fang, X.; Yu, D.; Wang, X. Challenges of SARS-CoV-2 Omicron Variant and Appropriate Countermeasures. Journal of Microbiology, Immunology and Infection 2022, 55 (3), 387-394. https://doi.org/10.1016/j.jmii.2022.03.007.
(16) Leuzinger, K.; Roloff, T.; Egli, A.; Hirsch, H. H. Impact of SARS-CoV-2 Omicron on Rapid Antigen Testing Developed for Early-Pandemic SARS-CoV-2 Variants. Microbiol Spectr 2022, 10 (4). https://doi.org/10.1128/spectrum.02006-22.
(17) United States Food and Drug Administration. At-Home COVID-19 Antigen Tests-Take Steps to Reduce Your Risk of False Negative: FDA Safety Communication.
(18) VanBlargan, L. A.; Errico, J. M.; Halfmann, P. J.; Zost, S. J.; Crowe, J. E.; Purcell, L. A.; Kawaoka, Y.; Corti, D.; Fremont, D. H.; Diamond, M. S. An Infectious SARS-CoV-2 B.1.1.529 Omicron Virus Escapes Neutralization by Therapeutic Monoclonal Antibodies. Nat Med 2022, 28 (3), 490-495. https://doi.org/10.1038/s41591-021-01678-y.
(19) Tuekprakhon, A.; Nutalai, R.; Dijokaite-Guraliuc, A.; Zhou, D.; Ginn, H. M.; Selvaraj, M.; Liu, C.; Mentzer, A. J.; Supasa, P.; Duyvesteyn, H. M. E.; Das, R.; Skelly, D.; Ritter, T. G.; Amini, A.; Bibi, S.; Adele, S.; Johnson, S. A.; Constantinides, B.; Webster, H.; Temperton, N.; Klenerman, P.; Barnes, E.; Dunachie, S. J.; Crook, D.; Pollard, A. J.; Lambe, T.; Goulder, P.; Paterson, N. G.; Williams, M. A.; Hall, D. R.; Fry, E. E.; Huo, J.; Mongkolsapaya, J.; Ren, J.; Stuart, D. I.; Screaton, G. R.; Conlon, C.; Deeks, A.; Frater, J.; Frending, L.; Gardiner, S.; Jämsen, A.; Jeffery, K.; Malone, T.; Phillips, E.; Rothwell, L.; Stafford, L. Antibody Escape of SARS-CoV-2 Omicron BA.4 and BA.5 from Vaccine and BA. 1 Serum. Cell 2022, 185 (14), 2422-2433.e13. https://doi.org/10.1016/j.cell.2022.06.005.
(20) Planas, D.; Saunders, N.; Maes, P.; Guivel-Benhassine, F.; Planchais, C.; Buchrieser, J.; Bolland, W.-H.; Porrot, F.; Staropoli, I.; Lemoine, F.; Péré, H.; Veyer, D.; Puech, J.; Rodary, J.; Baele, G.; Dellicour, S.; Raymenants, J.; Gorissen, S.; Geenen, C.; Vanmechelen, B.; Wawina-Bokalanga, T.; Martí-Carreras, J.; Cuypers, L.; Sève, A.; Hocqueloux, L.; Prazuck, T.; Rey, F. A.; Simon-Loriere, E.; Bruel, T.; Mouquet, H.; André, E.; Schwartz, O. Considerable Escape of SARS-CoV-2 Omicron to Antibody Neutralization. Nature 2022, 602 (7898), 671-675. https://doi.org/10.1038/s41586-021-04389-z.
(21) Hoffmann, M.; Zhang, L.; Pöhlmann, S. Omicron: Master of Immune Evasion Maintains Robust ACE2 Binding. Signal Transduct Target Ther 2022, 7 (1), 118. https://doi.org/10.1038/s41392-022-00965-5.
(22) Socher, E.; Heger, L.; Paulsen, F.; Zunke, F.; Arnold, P. Molecular Dynamics Simulations of the Delta and Omicron SARS-CoV-2 Spike-ACE2 Complexes Reveal Distinct Changes between Both Variants. Comput Struct Biotechnol J 2022, 20, 1168-1176. https://doi.org/10.1016/j.csbj.2022.02.015.
(23) Cotten, M.; Phan, M. V. T. Evolution to Increased Positive Charge on the Viral Spike Protein May Be Part of the Adaptation of SARS-CoV-2 to Human Transmission. bioRxiv 2022, 2022.07.30.502143. https://doi.org/10.1101/2022.07.30.502143.
(24) Mehta, P.; Ravi, V.; Devi, P.; Maurya, R.; Parveen, S.; Mishra, P.; Yadav, A.; Swaminathan, A.; Saifi, S.; Khare, K.; Chattopadhyay, P.; Yadav, M.; Chauhan, N. S.; Tarai, B.; Budhiraja, S.; Shamim, U.; Pandey, R. Mutational Dynamics across VOCs in International Travellers and Community Transmission Underscores Importance of Spike-ACE2 Interaction. Microbiol Res 2022, 262, 127099. https://doi.org/10.1016/j.micres.2022.127099.
(25) da Costa, C. H. S.; de Freitas, C. A. B.; Alves, C. N.; Lameira, J. Assessment of Mutations on RBD in the Spike Protein of SARS-CoV-2 Alpha, Delta and Omicron Variants. Sci Rep 2022, 12 (1), 8540. https://doi.org/10.1038/s41598-022-12479-9.
(26) Kim, S.; Liu, Y.; Ziarnik, M.; Cao, Y.; Zhang, X. F.; Im, W. Binding of Human ACE2 and RBD of Omicron Enhanced by Unique Interaction Patterns Among SARS-CoV-2 Variants of Concern. bioRxiv 2022, 2022.01.24.477633. https://doi.org/10.1101/2022.01.24.477633.
(27) Clausen, T. M.; Sandoval, D. R.; Spliid, C. B.; Pihl, J.; Perrett, H. R.; Painter, C. D.; Narayanan, A.; Majowicz, S. A.; Kwong, E. M.; McVicar, R. N.; Thacker, B. E.; Glass, C. A.; Yang, Z.; Torres, J. L.; Golden, G. J.; Bartels, P. L.; Porell, R. N.; Garretson, A. F.; Laubach, L.; Feldman, J.; Yin, X.; Pu, Y.; Hauser, B. M.; Caradonna, T. M.; Kellman, B. P.; Martino, C.; Gordts, P. L. S. M.; Chanda, S. K.; Schmidt, A. G.; Godula, K.; Leibel, S. L.; Jose, J.; Corbett, K. D.; Ward, A. B.; Carlin, A. F.; Esko, J. D. SARS-CoV-2 Infection Depends on Cellular Heparan Sulfate and ACE2. Cell 2020, 183 (4), 1043-1057.e15. https://doi.org/https://doi.org/10.1016/j.cell.2020.09.033.
(28) Yue, J.; Jin, W.; Yang, H.; Faulkner, J.; Song, X.; Qiu, H.; Teng, M.; Azadi, P.; Zhang, F.; Linhardt, R. J.; Wang, L. Heparan Sulfate Facilitates Spike Protein-Mediated SARS-CoV-2 Host Cell Invasion and Contributes to Increased Infection of SARS-CoV-2 G614 Mutant and in Lung Cancer. Front Mol Biosci 2021, 8. https://doi.org/10.3389/fmolb.2021.649575.
(29) Cagno; Tseligka; Jones; Tapparel. Heparan Sulfate Proteoglycans and Viral Attachment: True Receptors or Adaptation Bias? Viruses 2019, 11 (7), 596. https://doi.org/10.3390/v11070596.
(30) Stencel-Baerenwald, J. E.; Reiss, K.; Reiter, D. M.; Stehle, T.; Dermody, T. S. The Sweet Spot: Defining Virus-Sialic Acid Interactions. Nat Rev Microbiol 2014, 12 (11), 739-749. https://doi.org/10.1038/nrmicro3346.
(31) Connell, B. J.; Lortat-Jacob, H. Human Immunodeficiency Virus and Heparan Sulfate: From Attachment to Entry Inhibition. Front Immunol 2013, 4. https://doi.org/10.3389/fimmu.2013.00385.
(32) Xu, D.; Esko, J. D. Demystifying Heparan Sulfate-Protein Interactions. Annu Rev Biochem 2014, 83 (1), 129-157. https://doi.org/10.1146/annurev-biochem-060713-035314.
(33) Casalino, L.; Dommer, A. C.; Gaieb, Z.; Barros, E. P.; Sztain, T.; Ahn, S.-H.; Trifan, A.; Brace, A.; Bogetti, A. T.; Clyde, A.; Ma, H.; Lee, H.; Turilli, M.; Khalid, S.; Chong, L. T.; Simmerling, C.; Hardy, D. J.; Maia, J. D.; Phillips, J. C.; Kurth, T.; Stern, A. C.; Huang, L.; McCalpin, J. D.; Tatineni, M.; Gibbs, T.; Stone, J. E.; Jha, S.; Ramanathan, A.; Amaro, R. E. AI-Driven Multiscale Simulations Illuminate Mechanisms of SARS-CoV-2 Spike Dynamics. Int J High Perform Comput Appl 2021, 35 (5), 432-451. https://doi.org/10.1177/10943420211006452.
(34) Mycroft-West, C. J.; Su, D.; Pagani, I.; Rudd, T. R.; Elli, S.; Gandhi, N. S.; Guimond, S. E.; Miller, G. J.; Meneghetti, M. C. Z.; Nader, H. B.; Li, Y.; Nunes, Q. M.; Procter, P.; Mancini, N.; Clementi, M.; Bisio, A.; Forsyth, N. R.; Ferro, V.; Turnbull, J. E.; Guerrini, M.; Fernig, D. G.; Vicenzi, E.; Yates, E. A.; Lima, M. A.; Skidmore, M. A. Heparin Inhibits Cellular Invasion by SARS-CoV-2: Structural Dependence of the Interaction of the Spike S1 Receptor-Binding Domain with Heparin. Thromb Haemost 2020, 120 (12), 1700-1715. https://doi.org/10.1055/s-0040-1721319.
(35) Liu, L.; Chopra, P.; Li, X.; Bouwman, K. M.; Tompkins, S. M.; Wolfert, M. A.; de Vries, R. P.; Boons, G.-J. Heparan Sulfate Proteoglycans as Attachment Factor for SARS-CoV-2. ACS Cent Sci 2021, 7 (6), 1009-1018. https://doi.org/10.1021/acscentsci.1c00010.
(36) Kim, S. Y.; Jin, W.; Sood, A.; Montgomery, D. W.; Grant, O. C.; Fuster, M. M.; Fu, L.; Dordick, J. S.; Woods, R. J.; Zhang, F.; Linhardt, R. J. Characterization of Heparin and Severe Acute Respiratory Syndrome-Related Coronavirus 2 (SARS-CoV-2) Spike Glycoprotein Binding Interactions. Antiviral Res 2020, 181, 104873. https://doi.org/10.1016/j.antiviral.2020.104873.
(37) Kalra, R. S.; Kandimalla, R. Engaging the Spikes: Heparan Sulfate Facilitates SARS-CoV-2 Spike Protein Binding to ACE2 and Potentiates Viral Infection. Signal Transduct Target Ther 2021, 6 (1), 39. https://doi.org/10.1038/s41392-021-00470-1.
(38) Kim, S. H.; Kearns, F. L.; Rosenfeld, M. A.; Casalino, L.; Papanikolas, M. J.; Simmerling, C.; Amaro, R. E.; Freeman, R. GlycoGrip: Cell Surface-Inspired Universal Sensor for Betacoronaviruses. ACS Cent Sci 2022, 8 (1), 22-42. https://doi.org/10.1021/acscentsci.1c01080.
(39) Schuurs, Z. P.; Hammond, E.; Elli, S.; Rudd, T. R.; Mycroft-West, C. J.; Lima, M. A.; Skidmore, M. A.; Karlsson, R.; Chen, Y.-H.; Bagdonaite, I.; Yang, Z.; Ahmed, Y. A.; Richard, D. J.; Turnbull, J.; Ferro, V.; Coombe, D. R.; Gandhi, N. S. Evidence of a Putative Glycosaminoglycan Binding Site on the Glycosylated SARS-CoV-2 Spike Protein N-Terminal Domain. Comput Struct Biotechnol J 2021, 19, 2806-2818. https://doi.org/10.1016/j.csbj.2021.05.002.
(40) Schuurs, Z. P.; Hammond, E.; Elli, S.; Rudd, T. R.; Mycroft-West, C. J.; Lima, M. A.; Skidmore, M. A.; Karlsson, R.; Chen, Y.-H.; Bagdonaite, I.; Yang, Z.; Ahmed, Y. A.; Richard, D. J.; Turnbull, J.; Ferro, V.; Coombe, D. R.; Gandhi, N. S. Evidence of a Putative Glycosaminoglycan Binding Site on the Glycosylated SARS-CoV-2 Spike Protein N-Terminal Domain. Comput Struct Biotechnol J 2021, 19, 2806-2818. https://doi.org/10.1016/j.csbj.2021.05.002.
(41) Tandon, R.; Sharp, J. S.; Zhang, F.; Pomin, V. H.; Ashpole, N. M.; Mitra, D.; McCandless, M. G.; Jin, W.; Liu, H.; Sharma, P.; Linhardt, R. J. Effective Inhibition of SARS-CoV-2 Entry by Heparin and Enoxaparin Derivatives. J Virol 2020, 95 (3). https://doi.org/10.1128/JVI.01987-20.
(42) Zhang, Q.; Chen, C. Z.; Swaroop, M.; Xu, M.; Wang, L.; Lee, J.; Wang, A. Q.; Pradhan, M.; Hagen, N.; Chen, L.; Shen, M.; Luo, Z.; Xu, X.; Xu, Y.; Huang, W.; Zheng, W.; Ye, Y. Heparan Sulfate Assists SARS-CoV-2 in Cell Entry and Can Be Targeted by Approved Drugs in Vitro. Cell Discov 2020, 6 (1), 80. https://doi.org/10.1038/s41421-020-00222-5.
(43) Paiardi, G.; Richter, S.; Oreste, P.; Urbinati, C.; Rusnati, M.; Wade, R. C. The Binding of Heparin to Spike Glycoprotein Inhibits SARS-CoV-2 Infection by Three Mechanisms. Journal of Biological Chemistry 2022, 298 (2), 101507. https://doi.org/10.1016/j.jbc.2021.101507.
(44) Milewska, A.; Nowak, P.; Owczarek, K.; Szczepanski, A.; Zarebski, M.; Hoang, A.; Berniak, K.; Wojarski, J.; Zeglen, S.; Baster, Z.; Rajfur, Z.; Pyrc, K. Entry of Human Coronavirus NL63 into the Cell. J Virol 2018, 92 (3). https://doi.org/10.1128/JVI.01933-17.
(45) Lang, J.; Yang, N.; Deng, J.; Liu, K.; Yang, P.; Zhang, G.; Jiang, C. Inhibition of SARS Pseudovirus Cell Entry by Lactoferrin Binding to Heparan Sulfate Proteoglycans. PLoS One 2011, 6 (8), e23710. https://doi.org/10.1371/journal.pone.0023710.
(46) Pascarella, S.; Ciccozzi, M.; Bianchi, M.; Benvenuto, D.; Cauda, R.; Cassone, A. The Electrostatic Potential of the Omicron Variant Spike Is Higher than in Delta and Delta-plus Variants: A Hint to Higher Transmissibility? J Med Virol 2022, 94 (4), 1277-1280. https://doi.org/10.1002/jmv.27528.
(47) Gan, H. H.; Zinno, J.; Piano, F.; Gunsalus, K. C. Omicron Spike Protein Has a Positive Electrostatic Surface That Promotes ACE2 Recognition and Antibody Escape. Frontiers in Virology 2022, 2. https://doi.org/10.3389/fviro.2022.894531.
(48) Nie, C.; Sahoo, A. K.; Netz, R. R.; Herrmann, A.; Ballauff, M.; Haag, R. Charge Matters: Mutations in Omicron Variant Favor Binding to Cells. ChemBioChem 2022, 23 (6). https://doi.org/10.1002/cbic.202100681.
(49) Kearns, F. L.; Sandoval, D. R.; Casalino, L.; Clausen, T. M.; Rosenfeld, M. A.; Spliid, C. B.; Amaro, R. E.; Esko, J. D. Spike-Heparan Sulfate Interactions in SARS-CoV-2 Infection. Curr Opin Struct Biol 2022, 76, 102439. https://doi.org/10.1016/j.sbi.2022.102439.
(50) Lan, J.; Ge, J.; Yu, J.; Shan, S.; Zhou, H.; Fan, S.; Zhang, Q.; Shi, X.; Wang, Q.; Zhang, L.; Wang, X. Structure of the SARS-CoV-2 Spike Receptor-Binding Domain Bound to the ACE2 Receptor. Nature 2020, 581 (7807), 215-220. https://doi.org/10.1038/s41586-020-2180-5.
(51) Yan, R.; Zhang, Y.; Li, Y.; Xia, L.; Guo, Y.; Zhou, Q. Structural Basis for the Recognition of SARS-CoV-2 by Full-Length Human ACE2. Science (1979) 2020, 367 (6485), 1444-1448. https://doi.org/10.1126/science.abb2762.
(52) Zhao, P.; Praissman, J. L.; Grant, O. C.; Cai, Y.; Xiao, T.; Rosenbalm, K. E.; Aoki, K.; Kellman, B. P.; Bridger, R.; Barouch, D. H.; Brindley, M. A.; Lewis, N. E.; Tiemeyer, M.; Chen, B.; Woods, R. J.; Wells, L. Virus-Receptor Interactions of Glycosylated SARS-CoV-2 Spike and Human ACE2 Receptor. Cell Host Microbe 2020, 28 (4), 586-601.e6. https://doi.org/10.1016/j.chom.2020.08.004.
(53) Barros, E. P.; Casalino, L.; Gaieb, Z.; Dommer, A. C.; Wang, Y.; Fallon, L.; Raguette, L.; Belfon, K.; Simmerling, C.; Amaro, R. E. The Flexibility of ACE2 in the Context of SARS-CoV-2 Infection. Biophys J 2021, 120 (6), 1072-1084. https://doi.org/https://doi.org/10.1016/j.bpj.2020.10.036.
(54) Ozono, S.; Zhang, Y.; Ode, H.; Sano, K.; Tan, T. S.; Imai, K.; Miyoshi, K.; Kishigami, S.; Ueno, T.; Iwatani, Y.; Suzuki, T.; Tokunaga, K. SARS-CoV-2 D614G Spike Mutation Increases Entry Efficiency with Enhanced ACE2-Binding Affinity. Nat Commun 2021, 12 (1), 848. https://doi.org/10.1038/s41467-021-21118-2.
(55) Verdecchia, P.; Cavallini, C.; Spanevello, A.; Angeli, F. The Pivotal Link between ACE2 Deficiency and SARS-CoV-2 Infection. Eur J Intern Med 2020, 76, 14-20. https://doi.org/10.1016/j.ejim.2020.04.037.
(56) Casalino, L.; Gaieb, Z.; Goldsmith, J. A.; Hjorth, C. K.; Dommer, A. C.; Harbison, A. M.; Fogarty, C. A.; Barros, E. P.; Taylor, B. C.; McLellan, J. S.; Fadda, E.; Amaro, R. E. Beyond Shielding: The Roles of Glycans in the SARS-CoV-2 Spike Protein. ACS Cent Sci 2020, 6 (10), 1722-1734. https://doi.org/10.1021/acscentsci.0c01056.
(57) Sztain, T.; Ahn, S.-H.; Bogetti, A. T.; Casalino, L.; Goldsmith, J. A.; Seitz, E.; McCool, R. S.; Kearns, F. L.; Acosta-Reyes, F.; Maji, S.; Mashayekhi, G.; McCammon, J. A.; Ourmazd, A.; Frank, J.; Mclellan, J. S.; Chong, L. T.; Amaro, R. E. A Glycan Gate Controls Opening of the SARS-CoV-2 Spike Protein. Nat Chem 2021, 2021.02.15.431212. https://doi.org/10.1038/s41557-021-00758-3.
(58) Walls, A. C.; Park, Y.-J.; Tortorici, M. A.; Wall, A.; McGuire, A. T.; Veesler, D. Structure, Function, and Antigenicity of the SARS-CoV-2 Spike Glycoprotein. Cell 2020, 181 (2), 281-292.e6. https://doi.org/10.1016/j.cell.2020.02.058.
(59) Wrapp, D.; Wang, N.; Corbett, K. S.; Goldsmith, J. A.; Hsieh, C.-L.; Abiona, O.; Graham, B. S.; Mclellan, J. S. Cryo-EM Structure of the 2019-NCOV Spike in the Prefusion Conformation. Science (1979) 2020, 367 (6483), 1260 LP-1263. https://doi.org/10.1126/science.abb2507.
(60) Rahbar Saadat, Y.; Hosseiniyan Khatibi, S. M.; Zununi Vahed, S.; Ardalan, M. Host Serine Proteases: A Potential Targeted Therapy for COVID-19 and Influenza. Front Mol Biosci 2021, 8. https://doi.org/10.3389/fmolb.2021.725528.
(61) Sasaki, M.; Uemura, K.; Sato, A.; Toba, S.; Sanaki, T.; Maenaka, K.; Hall, W. W.; Orba, Y.; Sawa, H. SARS-CoV-2 Variants with Mutations at the S1/S2 Cleavage Site Are Generated in Vitro during Propagation in TMPRSS2-Deficient Cells. PLoS Pathog 2021, 17 (1), e1009233. https://doi.org/10.1371/journal.ppat.1009233.
(62) Papa, G.; Mallery, D. L.; Albecka, A.; Welch, L. G.; Cattin-Ortolá, J.; Luptak, J.; Paul, D.; McMahon, H. T.; Goodfellow, I. G.; Carter, A.; Munro, S.; James, L. C. Furin Cleavage of SARS-CoV-2 Spike Promotes but Is Not Essential for Infection and Cell-Cell Fusion. PLoS Pathog 2021, 17 (1), e1009246. https://doi.org/10.1371/journal.ppat.1009246.
(63) Essalmani, R.; Jain, J.; Susan-Resiga, D.; Andréo, U.; Evagelidis, A.; Derbali, R. M.; Huynh, D. N.; Dallaire, F.; Laporte, M.; Delpal, A.; Sutto-Ortiz, P.; Coutard, B.; Mapa, C.; Wilcoxen, K.; Decroly, E.; NQ Pham, T.; Cohen, É. A.; Seidah, N. G. Distinctive Roles of Furin and TMPRSS2 in SARS-CoV-2 Infectivity. J Virol 2022, 96 (8). https://doi.org/10.1128/jvi.00128-22.
(64) Bestle, D.; Heindl, M. R.; Limburg, H.; van Lam van, T.; Pilgram, O.; Moulton, H.; Stein, D. A.; Hardes, K.; Eickmann, M.; Dolnik, O.; Rohde, C.; Klenk, H.-D.; Garten, W.; Steinmetzer, T.; Böttcher-Friebertshäuser, E. TMPRSS2 and Furin Are Both Essential for Proteolytic Activation of SARS-CoV-2 in Human Airway Cells. Life Sci Alliance 2020, 3 (9), e202000786. https://doi.org/10.26508/lsa.202000786.
(65) Koppisetti, R. K.; Fulcher, Y. G.; van Doren, S. R. Fusion Peptide of SARS-CoV-2 Spike Rearranges into a Wedge Inserted in Bilayered Micelles. J Am Chem Soc 2021, 143 (33), 13205-13211. https://doi.org/10.1021/jacs.1c05435.
(66) Jackson, C. B.; Farzan, M.; Chen, B.; Choe, H. Mechanisms of SARS-CoV-2 Entry into Cells. Nat Rev Mol Cell Biol 2022, 23 (1), 3-20. https://doi.org/10.1038/s41580-021-00418-X.
(67) Shang, J.; Wan, Y.; Luo, C.; Ye, G.; Geng, Q.; Auerbach, A.; Li, F. Cell Entry Mechanisms of SARS-CoV-2. Proceedings of the National Academy of Sciences 2020, 117 (21), 11727-11734. https://doi.org/10.1073/pnas.2003138117.
(68) Meng, B.; Abdullahi, A.; Ferreira, I. A. T. M.; Goonawardane, N.; Saito, A.; Kimura, I.; Yamasoba, D.; Gerber, P. P.; Fatihi, S.; Rathore, S.; Zepeda, S. K.; Papa, G.; Kemp, S. A.; Ikeda, T.; Toyoda, M.; Tan, T. S.; Kuramochi, J.; Mitsunaga, S.; Ueno, T.; Shirakawa, K.; Takaori-Kondo, A.; Brevini, T.; Mallery, D. L.; Charles, O. J.; Baker, S.; Dougan, G.; Hess, C.; Kingston, N.; Lehner, P. J.; Lyons, P. A.; Matheson, N. J.; Ouwehand, W. H.; Saunders, C.; Summers, C.; Thaventhiran, J. E. D.; Toshner, M.; Weekes, M. P.; Maxwell, P.; Shaw, A.; Bucke, A.; Calder, J.; Canna, L.; Domingo, J.; Elmer, A.; Fuller, S.; Harris, J.; Hewitt, S.; Kennet, J.; Jose, S.; Kourampa, J.; Meadows, A.; O'Brien, C.; Price, J.; Publico, C.; Rastall, R.; Ribeiro, C.; Rowlands, J.; Ruffolo, V.; Tordesillas, H.; Bullman, B.; Dunmore, B. J.; Gräf, S.; Hodgson, J.; Huang, C.; Hunter, K.; Jones, E.; Legchenko, E.; Matara, C.; Martin, J.; Mescia, F.; O'Donnell, C.; Pointon, L.; Shih, J.; Sutcliffe, R.; Tilly, T.; Treacy, C.; Tong, Z.; Wood, J.; Wylot, M.; Betancourt, A.; Bower, G.; Cossetti, C.; de Sa, A.; Epping, M.; Fawke, S.; Gleadall, N.; Grenfell, R.; Hinch, A.; Jackson, S.; Jarvis, I.; Krishna, B.; Nice, F.; Omarjee, O.; Perera, M.; Potts, M.; Richoz, N.; Romashova, V.; Stefanucci, L.; Strezlecki, M.; Turner, L.; de Bie, E. M. D. D.; Bunclark, K.; Josipovic, M.; Mackay, M.; Butcher, H.; Caputo, D.; Chandler, M.; Chinnery, P.; Clapham-Riley, D.; Dewhurst, E.; Fernandez, C.; Furlong, A.; Graves, B.; Gray, J.; Hein, S.; Ivers, T.; l Gresley, E.; Linger, R.; Kasanicki, M.; King, R.; Kingston, N.; Meloy, S.; Moulton, A.; Muldoon, F.; Ovington, N.; Papadia, S.; Penkett, C. J.; Phelan, I.; Ranganath, V.; Paraschiv, R.; Sage, A.; Sambrook, J.; Scholtes, I.; Schon, K.; Stark, H.; Stirrups, K. E.; Townsend, P.; Walker, N.; Webster, J.; Butlertanaka, E. P.; Tanaka, Y. L.; Ito, J.; Uriu, K.; Kosugi, Y.; Suganami, M.; Oide, A.; Yokoyama, M.; Chiba, M.; Motozono, C.; Nasser, H.; Shimizu, R.; Kitazato, K.; Hasebe, H.; Irie, T.; Nakagawa, S.; Wu, J.; Takahashi, M.; Fukuhara, T.; Shimizu, K.; Tsushima, K.; Kubo, H.; Kazuma, Y.; Nomura, R.; Horisawa, Y.; Nagata, K.; Kawai, Y.; Yanagida, Y.; Tashiro, Y.; Tokunaga, K.; Ozono, S.; Kawabata, R.; Morizako, N.; Sadamasu, K.; Asakura, H.; Nagashima, M.; Yoshimura, K.; Cárdenas, P.; Muñoz, E.; Barragan, V.; Márquez, S.; Prado-Vivar, B.; Becerra-Wong, M.; Caravajal, M.; Trueba, G.; Rojas-Silva, P.; Grunauer, M.; Gutierrez, B.; Guadalupe, J. J.; Fernández-Cadena, J. C.; Andrade-Molina, D.; Baldeon, M.; Pinos, A.; Bowen, J. E.; Joshi, A.; Walls, A. C.; Jackson, L.; Martin, D.; Smith, K. G. C.; Bradley, J.; Briggs, J. A. G.; Choi, J.; Madissoon, E.; Meyer, K. B.; Mlcochova, P.; Ceron-Gutierrez, L.; Doffinger, R.; Teichmann, S. A.; Fisher, A. J.; Pizzuto, M. S.; de Marco, A.; Corti, D.; Hosmillo, M.; Lee, J. H.; James, L. C.; Thukral, L.; Veesler, D.; Sigal, A.; Sampaziotis, F.; Goodfellow, I. G.; Matheson, N. J.; Sato, K.; Gupta, R. K. Altered TMPRSS2 Usage by SARS-CoV-2 Omicron Impacts Infectivity and Fusogenicity. Nature 2022, 603 (7902), 706-714. https://doi.org/10.1038/s41586-022-04474-x.
(69) Willett, B. J.; Grove, J.; MacLean, O. A.; Wilkie, C.; Logan, N.; de Lorenzo, G.; Furnon, W.; Scott, S.; Manali, M.; Szemiel, A.; Ashraf, S.; Vink, E.; Harvey, W. T.; Davis, C.; Orton, R.; Hughes, J.; Holland, P.; Silva, V.; Pascall, D.; Puxty, K.; da Silva Filipe, A.; Yebra, G.; Murcia, P. R.; Patel, A. H.; The COVID-19 Genomics UK (COG-UK) Consortium; Haughney, J.; Robertson, D. L.; Palmarini, M.; Ray, S.; Thomson, E. C. The Hyper-Transmissible SARS-CoV-2 Omicron Variant Exhibits Significant Antigenic Change, Vaccine Escape and a Switch in Cell Entry Mechanism. medRxiv 2022.
(70) Peacock, T. P.; Brown, J. C.; Zhou, J.; Thakur, N.; Sukhova, K.; Newman, J.; Kugathasan, R.; Yan, A. W. C.; Furnon, W.; de Lorenzo, G.; Cowton, V. M.; Reuss, D.; Moshe, M.; Quantrill, J. L.; Platt, O. K.; Kaforou, M.; Patel, A. H.; Palmarini, M.; Bailey, D.; Barclay, W. S. The Altered Entry Pathway and Antigenic Distance of the SARS-CoV-2 Omicron Variant Map to Separate Domains of Spike Protein. bioRxiv 2022, 2021.12.31.474653. https://doi.org/10.1101/2021.12.31.474653.
(71) Wang, X.; Bie, L.; Gao, J. Structural Insights into the Cofactor Role of Heparin/Heparan Sulfate in Binding between the SARS-CoV-2 Spike Protein and Host Angiotensin-Converting Enzyme II. J Chem Inf Model 2022, 62 (3), 656-667. https://doi.org/10.1021/acs.jcim.1c01484.
(72) Han, P.; Li, L.; Liu, S.; Wang, Q.; Zhang, D.; Xu, Z.; Han, P.; Li, X.; Peng, Q.; Su, C.; Huang, B.; Li, D.; Zhang, R.; Tian, M.; Fu, L.; Gao, Y.; Zhao, X.; Liu, K.; Qi, J.; Gao, G. F.; Wang, P. Receptor Binding and Complex Structures of Human ACE2 to Spike RBD from Omicron and Delta SARS-CoV-2. Cell 2022, 185 (4), 630-640.e10. https://doi.org/10.1016/j.cell.2022.01.001.
(73) Trott, O.; Olson, A. J. AutoDock Vina: Improving the Speed and Accuracy of Docking with a New Scoring Function, Efficient Optimization, and Multithreading. J Comput Chem 2010, 31 (2), 455-461. https://doi.org/https://doi.org/10.1002/jcc.21334.
(74) Morris, G. M.; Huey, R.; Lindstrom, W.; Sanner, M. F.; Belew, R. K.; Goodsell, D. S.; Olson, A. J. AutoDock4 and AutoDockTools4: Automated Docking with Selective Receptor Flexibility. J Comput Chem 2009, 30 (16), 2785-2791. https://doi.org/https://doi.org/10.1002/jcc.21256.
(75) Schrodinger Release 2022-3. Induced Fit Docking Protocol. Schr {\ “o} dinger, LLC: New York, NY 2022.
(76) Sherman, W.; Beard, H. S.; Farid, R. Use of an Induced Fit Receptor Structure in Virtual Screening. Chemical Biology <html_ent glyph=” @amp;” ascii=“&amp;”/>Drug Design 2006, 67 (1), 83-84. https://doi.org/10.1111/j.1747-0285.2005.00327.x.
(77) Sherman, W.; Day, T.; Jacobson, M. P.; Friesner, R. A.; Farid, R. Novel Procedure for Modeling Ligand/Receptor Induced Fit Effects. J Med Chem 2006, 49 (2), 534-553. https://doi.org/10.1021/jm050540c.
(78) Farid, R.; Day, T.; Friesner, R. A.; Pearlstein, R. A. New Insights about HERG Blockade Obtained from Protein Modeling, Potential Energy Mapping, and Docking Studies. Bioorg Med Chem 2006, 14 (9), 3160-3173. https://doi.org/10.1016/j.bmc.2005.12.032.
(79) Schrödinger Release 2021-3. Glide. Schrödinger, LLC: New York, NY 2021.
(80) Zimmer, C.; Corum, J. Coronavirus in a Tiny Drop. New York Times. 2021.
(81) Cotten, M.; Phan, M. V. T. Evolution to Increased Positive Charge on the Viral Spike Protein May Be Part of the Adaptation of SARS-CoV-2 to Human Transmission. bioRxiv 2022, 2022.07.30.502143. https://doi.org/10.1101/2022.07.30.502143.
(82) Watanabe, Y.; Allen, J. D.; Wrapp, D.; Mclellan, J. S.; Crispin, M. Site-Specific Glycan Analysis of the SARS-CoV-2 Spike. Science (1979) 2020, 369 (6501), 330-333. https://doi.org/10.1126/science.abb9983.
(83) Olsson, M. H. M.; Søndergaard, C. R.; Rostkowski, M.; Jensen, J. H. PROPKA3: Consistent Treatment of Internal and Surface Residues in Empirical PKa Predictions. J Chem Theory Comput 2011, 7 (2), 525-537. https://doi.org/10.1021/ct100578z.
(84) Huber, G. A.; McCammon, J. A. Brownian Dynamics Simulations of Biological Molecules. Trends Chem 2019, 1 (8), 727-738. https://doi.org/10.1016/j.trechm.2019.07.008.
(85) Huber, G. A.; McCammon, J. A. Browndye: A Software Package for Brownian Dynamics. Comput Phys Commun 2010, 181 (11), 1896-1905. https://doi.org/10.1016/j.cpc.2010.07.022.
(86) Chavanis, P.-H. The Generalized Stochastic Smoluchowski Equation. Entropy 2019, 21 (10), 1006. https://doi.org/10.3390/e21101006.
(87) Mannar, D.; Saville, J. W.; Zhu, X.; Srivastava, S. S.; Berezuk, A. M.; Tuttle, K. S.; Marquez, A. C.; Sekirov, I.; Subramaniam, S. SARS-CoV-2 Omicron Variant: Antibody Evasion and Cryo-EM Structure of Spike Protein-ACE2 Complex. Science (1979) 2022, 375 (6582), 760-764. https://doi.org/10.1126/science.abn7760.
(88) Xiao, T.; Lu, J.; Zhang, J.; Johnson, R. I.; Mckay, L. G. A.; Storm, N.; Lavine, C. L.; Peng, H.; Cai, Y.; Rits-Volloch, S.; Lu, S.; Quinlan, B. D.; Farzan, M.; Seaman, M. S.; Griffiths, A.; Chen, B. A Trimeric Human Angiotensin-Converting Enzyme 2 as an Anti-SARS-CoV-2 Agent. Nat Struct Mol Biol 2021, 28 (2), 202-209. https://doi.org/10.1038/s41594-020-00549-3.
(89) Kim, S. H.; Kearns, F. L.; Rosenfeld, M. A.; Casalino, L.; Papanikolas, M. J.; Simmerling, C.; Amaro, R. E.; Freeman, R. GlycoGrip: Cell Surface-Inspired Universal Sensor for Betacoronaviruses. ACS Cent Sci 2021.
(90) Amaro, R. E.; Mulholland, A. J. A Community Letter Regarding Sharing Biomolecular Simulation Data for COVID-19. J Chem Inf Model 2020, 60 (6), 2653-2656. https://doi.org/10.1021/acs.jcim.0c00319.
(91) Amaro, R. E.; Mulholland, A. J. Biomolecular Simulations in the Time of COVID-19, and After. Comput Sci Eng 2020, 22 (6), 30-36. https://doi.org/10.1109/MCSE.2020.3024155.
(92) Mulholland, A. J.; Amaro, R. E. COVID19-Computational Chemists Meet the Moment. J Chem Inf Model 2020, 60 (12), 5724-5726. https://doi.org/10.1021/acs.jcim.0c01395.
(93) Walls, A. C.; Park, Y.-J.; Tortorici, M. A.; Wall, A.; McGuire, A. T.; Veesler, D. Structure, Function, and Antigenicity of the SARS-CoV-2 Spike Glycoprotein. Cell 2020, 181 (2), 281-292.e6. https://doi.org/10.1016/j.cell.2020.02.058.
(94) Bangaru, S.; Ozorowski, G.; Turner, H. L.; Antanasijevic, A.; Huang, D.; Wang, X.; Torres, J. L.; Diedrich, J. K.; Tian, J.-H.; Portnoff, A. D.; Patel, N.; Massare, M. J.; Yates, J. R.; Nemazee, D.; Paulson, J. C.; Glenn, G.; Smith, G.; Ward, A. B. Structural Analysis of Full-Length SARS-CoV-2 Spike Protein from an Advanced Vaccine Candidate. Science (1979) 2020, 370 (6520), 1089 LP-1094. https://doi.org/10.1126/science.abe1502.
(95) Gobeil, S. M.-C.; Henderson, R.; Stalls, V.; Janowska, K.; Huang, X.; May, A.; Speakman, M.; Beaudoin, E.; Manne, K.; Li, D.; Parks, R.; Barr, M.; Deyton, M.; Martin, M.; Mansouri, K.; Edwards, R. J.; Eaton, A.; Montefiori, D. C.; Sempowski, G. D.; Saunders, K. O.; Wiehe, K.; Williams, W.; Korber, B.; Haynes, B. F.; Acharya, P. Structural Diversity of the SARS-CoV-2 Omicron Spike. Mol Cell 2022, 82 (11), 2050-2068.e6. https://doi.org/10.1016/j.molcel.2022.03.028.
(96) Tortorici, M. A.; Beltramello, M.; Lempp, F. A.; Pinto, D.; Dang, H. v.; Rosen, L. E.; McCallum, M.; Bowen, J.; Minola, A.; Jaconi, S.; Zatta, F.; de Marco, A.; Guarino, B.; Bianchi, S.; Lauron, E. J.; Tucker, H.; Zhou, J.; Peter, A.; Havenar-Daughton, C.; Wojcechowskyj, J. A.; Case, J. B.; Chen, R. E.; Kaiser, H.; Montiel-Ruiz, M.; Meury, M.; Czudnochowski, N.; Spreafico, R.; Dillen, J.; Ng, C.; Sprugasci, N.; Culap, K.; Benigni, F.; Abdelnabi, R.; Foo, S.-Y. C.; Schmid, M. A.; Cameroni, E.; Riva, A.; Gabrieli, A.; Galli, M.; Pizzuto, M. S.; Neyts, J.; Diamond, M. S.; Virgin, H. W.; Snell, G.; Corti, D.; Fink, K.; Veesler, D. Ultrapotent Human Antibodies Protect against SARS-CoV-2 Challenge via Multiple Mechanisms. Science (1979) 2020, 370 (6519), 950-957. https://doi.org/10.1126/science.abe3354.
(97) Casalino, L.; Gaieb, Z.; Goldsmith, J. A.; Hjorth, C. K.; Dommer, A. C.; Harbison, A. M.; Fogarty, C. A.; Barros, E. P.; Taylor, B. C.; Mclellan, J. S.; Fadda, E.; Amaro, R. E. Beyond Shielding: The Roles of Glycans in the SARS-CoV-2 Spike Protein. ACS Cent Sci 2020, 6 (10), 1722-1734. https://doi.org/10.1021/acscentsci.0c01056.
(98) Watanabe, Y.; Allen, J. D.; Wrapp, D.; Mclellan, J. S.; Crispin, M. Site-Specific Glycan Analysis of the SARS-CoV-2 Spike. Science (1979) 2020, 369 (6501), 330-333. https://doi.org/10.1126/science.abb9983.
(99) Olsson, M. H. M.; Søndergaard, C. R.; Rostkowski, M.; Jensen, J. H. PROPKA3: Consistent Treatment of Internal and Surface Residues in Empirical PKa Predictions. J Chem Theory Comput 2011, 7 (2), 525-537. https://doi.org/10.1021/ct100578z.
(100) Schrödinger Release 2021-3. Protein Preparation Wizard. Schrödinger, LLC: New York, NY 2021.
(101) Humphrey, W.; Dalke, A.; Schulten, K. VMD: Visual Molecular Dynamics. J Mol Graph 1996, 14 (1), 33-38. https://doi.org/https://doi.org/10.1016/0263-7855 (96) 00018-5.
(102) Phillips, J. C.; Braun, R.; Wang, W.; Gumbart, J.; Tajkhorshid, E.; Villa, E.; Chipot, C.; Skeel, R. D.; Kalé, L.; Schulten, K. Scalable Molecular Dynamics with NAMD. J Comput Chem 2005, 26 (16), 1781-1802. https://doi.org/10.1002/jcc.20289.
(103) Phillips, J. C.; Hardy, D. J.; Maia, J. D. C.; Stone, J. E.; Ribeiro, J. v; Bernardi, R. C.; Buch, R.; Fiorin, G.; Hénin, J.; Jiang, W.; McGreevy, R.; Melo, M. C. R.; Radak, B. K.; Skeel, R. D.; Singharoy, A.; Wang, Y.; Roux, B.; Aksimentiev, A.; Luthey-Schulten, Z.; Kalé, L. v; Schulten, K.; Chipot, C.; Tajkhorshid, E. Scalable Molecular Dynamics on CPU and GPU Architectures with NAMD. J Chem Phys 2020, 153 (4), 44130. https://doi.org/10.1063/5.0014475.
(104) Huang, J.; Mackerell, A. D. CHARMM36 All-Atom Additive Protein Force Field: Validation Based on Comparison to NMR Data. J Comput Chem 2013, 34 (25), 2135-2145. https://doi.org/10.1002/jcc.23354.
(105) Huang, J.; Rauscher, S.; Nawrocki, G.; Ran, T.; Feig, M.; de Groot, B. L.; Grubmüller, H.; Mackerell, A. D. CHARMM36m: An Improved Force Field for Folded and Intrinsically Disordered Proteins. Nat Methods 2017, 14 (1), 71-73. https://doi.org/10.1038/nmeth.4067.
(106) Trott, O.; Olson, A. J. AutoDock Vina: Improving the Speed and Accuracy of Docking with a New Scoring Function, Efficient Optimization, and Multithreading. J Comput Chem 2010, 31 (2), 455-461. https://doi.org/https://doi.org/10.1002/jcc.21334.
(107) Morris, G. M.; Huey, R.; Lindstrom, W.; Sanner, M. F.; Belew, R. K.; Goodsell, D. S.; Olson, A. J. AutoDock4 and AutoDockTools4: Automated Docking with Selective Receptor Flexibility. J Comput Chem 2009, 30 (16), 2785-2791. https://doi.org/https://doi.org/10.1002/jcc.21256.
(108) Gowers, R.; Linke, M.; Barnoud, J.; Reddy, T.; Melo, M.; Seyler, S.; Domański, J.; Dotson, D.; Buchoux, S.; Kenney, I.; Beckstein, O. MDAnalysis: A Python Package for the Rapid Analysis of Molecular Dynamics Simulations; 2016; pp 98-105. https://doi.org/10.25080/Majora-629e541a-00e.
(109) Michaud-Agrawal, N.; Denning, E. J.; Woolf, T. B.; Beckstein, O. MDAnalysis: A Toolkit for the Analysis of Molecular Dynamics Simulations. J Comput Chem 2011, 32 (10), 2319-2327. https://doi.org/10.1002/jcc.21787.
(18) Pedregosa, F.; Varoquaux, G.; Gramfort, A.; Michel, V.; Thirion, B.; Grisel, O.; Blondel, M.; Prettenhofer, P.; Weiss, R.; Dubourg, V.; Vanderplas, J.; Passos, A.; Cournapeau, D.; Brucher, M.; Perrot, M.; Duchesnay, E. Scikit-Learn: Machine Learning in Python. Journal of Machine Learning Research 2011, 12 (85), 2825-2830.
(110) Kim, S. H.; Kearns, F. L.; Rosenfeld, M. A.; Casalino, L.; Papanikolas, M. J.; Simmerling, C.; Amaro, R. E.; Freeman, R. GlycoGrip: Cell Surface-Inspired Universal Sensor for Betacoronaviruses. ACS Cent Sci 2021.
(112) Shrake, A.; Rupley, J. A. Environment and Exposure to Solvent of Protein Atoms. Lysozyme and Insulin. J Mol Biol 1973, 79 (2), 351-371. https://doi.org/https://doi.org/10.1016/0022-2836 (73) 90011-9.
(113) Schrodinger Release 2022-3. Induced Fit Docking Protocol. Schr {\″o} dinger, LLC: New York, NY 2022.
(114) Lu, C.; Wu, C.; Ghoreishi, D.; Chen, W.; Wang, L.; Damm, W.; Ross, G. A.; Dahlgren, M. K.; Russell, E.; von Bargen, C. D.; Abel, R.; Friesner, R. A.; Harder, E. D. OPLS4: Improving Force Field Accuracy on Challenging Regimes of Chemical Space. J Chem Theory Comput 2021, 17 (7), 4291-4300. https://doi.org/10.1021/acs.jctc.1c00302.
(115) Schrödinger Release 2021-3. LigPrep. Schrödinger, LLC: New York, NY 2021.
(116) Dolinsky, T. J.; Nielsen, J. E.; McCammon, J. A.; Baker, N. A. PDB2PQR: An Automated Pipeline for the Setup of Poisson-Boltzmann Electrostatics Calculations. Nucleic Acids Res 2004, 32 (Web Server), W665-W667. https://doi.org/10.1093/nar/gkh381.
(117) Dolinsky, T. J.; Czodrowski, P.; Li, H.; Nielsen, J. E.; Jensen, J. H.; Klebe, G.; Baker, N. A. PDB2PQR: Expanding and Upgrading Automated Preparation of Biomolecular Structures for Molecular Simulations. Nucleic Acids Res 2007, 35 (Web Server), W522-W525. https://doi.org/10.1093/nar/gkm276.
(118) Huber, G. A.; McCammon, J. A. Browndye: A Software Package for Brownian Dynamics. Comput Phys Commun 2010, 181 (11), 1896-1905. https://doi.org/10.1016/j.cpc.2010.07.022.
(119) Jurrus, E.; Engel, D.; Star, K.; Monson, K.; Brandi, J.; Felberg, L. E.; Brookes, D. H.; Wilson, L.; Chen, J.; Liles, K.; Chun, M.; Li, P.; Gohara, D. W.; Dolinsky, T.; Konecny, R.; Koes, D. R.; Nielsen, J. E.; Head-Gordon, T.; Geng, W.; Krasny, R.; Wei, G.-W.; Holst, M. J.; McCammon, J. A.; Baker, N. A. Improvements to the APBS Biomolecular Solvation Software Suite. Protein Science 2018, 27 (1), 112-128. https://doi.org/https://doi.org/10.1002/pro.3280.
(120) Baker, N. A.; Sept, D.; Joseph, S.; Holst, M. J.; McCammon, J. A. Electrostatics of Nanosystems: Application to Microtubules and the Ribosome. Proceedings of the National Academy of Sciences 2001, 98 (18), 10037 LP-10041. https://doi.org/10.1073/pnas. 181342398.
(121) Holst, M. Adaptive Numerical Treatment of Elliptic Systems on Manifolds. Adv Comput Math 2001, 15 (1), 139-191. https://doi.org/10.1023/A: 1014246117321.
(122) Holst, M. J.; Saied, F. Numerical Solution of the Nonlinear Poisson-Boltzmann Equation: Developing More Robust and Efficient Methods. J Comput Chem 1995, 16 (3), 337-364. https://doi.org/https://doi.org/10.1002/jcc.540160308.
(123) Holst, M.; Saied, F. Multigrid Solution of the Poisson-Boltzmann Equation. J Comput Chem 1993, 14 (1), 105-113. https://doi.org/https://doi.org/10.1002/jcc.540140114.
(124) Bank, R. E.; Holst, M. A New Paradigm for Parallel Adaptive Meshing Algorithms. SIAM Review 2003, 45 (2), 291-323. https://doi.org/10.1137/S003614450342061.
(125) Votapka, L. W.; Amaro, R. E. Multiscale Estimation of Binding Kinetics Using Brownian Dynamics, Molecular Dynamics and Milestoning. PLoS Comput Biol 2015, 11 (10), e1004381. https://doi.org/10.1371/journal.pcbi.1004381.
(126) Chavanis, P.-H. The Generalized Stochastic Smoluchowski Equation. Entropy 2019, 21 (10), 1006. https://doi.org/10.3390/e21101006.
(127) Yan, R.; Zhang, Y.; Li, Y.; Xia, L.; Guo, Y.; Zhou, Q. Structural Basis for the Recognition of SARS-CoV-2 by Full-Length Human ACE2. Science (1979) 2020, 367 (6485), 1444-1448. https://doi.org/10.1126/science.abb2762.
(128) Barros, E. P.; Casalino, L.; Gaieb, Z.; Dommer, A. C.; Wang, Y.; Fallon, L.; Raguette, L.; Belfon, K.; Simmerling, C.; Amaro, R. E. The Flexibility of ACE2 in the Context of SARS-CoV-2 Infection. Biophys J 2021, 120 (6), 1072-1084. https://doi.org/10.1016/j.bpj.2020.10.036.
(129) Rodríguez, M. O.; Covián, L. B.; García, A. C.; Blanco-López, M. C. Silver and Gold Enhancement Methods for Lateral Flow Immunoassays. Talanta 2016, 148, 272-278. https://doi.org/10.1016/j.talanta.2015.10.068.
(130) Casalino, L.; Dommer, A. C.; Gaieb, Z.; Barros, E. P.; Sztain, T.; Ahn, S.-H.; Trifan, A.; Brace, A.; Bogetti, A. T.; Clyde, A.; Ma, H.; Lee, H.; Turilli, M.; Khalid, S.; Chong, L. T.; Simmerling, C.; Hardy, D. J.; Maia, J. D.; Phillips, J. C.; Kurth, T.; Stern, A. C.; Huang, L.; McCalpin, J. D.; Tatineni, M.; Gibbs, T.; Stone, J. E.; Jha, S.; Ramanathan, A.; Amaro, R. E. AI-Driven Multiscale Simulations Illuminate Mechanisms of SARS-CoV-2 Spike Dynamics. Int J High Perform Comput Appl 2021, 35 (5), 432-451. https://doi.org/10.1177/10943420211006452.

### Example 4

HIV is a highly researched area, with the US government spending Billions of Dollars each year to eradicate the burden of this disease on the US through federal funding; and globally through The U.S. President's Emergency Plan for AIDS Relief (PEPFAR), spending over $100 billion since its launch in 2003. Still, HIV remains a great threat to global health. Currently, approximately 38 million people are living with HIV worldwide and there are approximately 1.5 million people newly infected every year. HIV infects the CD4 cells and progressively destroys these CD4 cells, leading to immune system failure. HIV infection can be divided into three stages: (1) Acute HIV infection; (2) Chronic HIV infection; and (3) Advanced HIV infection. (1) Acute infection is the earliest stage after onset of the infection, when HIV rapidly multiplies and spreads throughout the body, increasing transmission risk. (2) During the chronic HIV infection stage, the HI-virus replication is reduced due to antibody production (seroconversion). (3) Finally, during the last stage, the advanced HIV infection stage, the number of CD4 cells are greatly reduced, causing immunodeficiency, known as AIDS.

Acute HIV infection is the most important stage to effectively treat and reduce the transmission of the virus by lowering the viral load using anti-retroviral treatment. In the US, at least 1 in 6 men who have sex with men (MSM) with HIV do not know their status and make up 66% of new annual infections. Therefore, early diagnosis of HIV infection is a key-step for effective treatment to reduce the spread. To achieve the early diagnosis, many detection methods including PCR, western blot, and ELISA have been developed, however, point-of-care testing using lateral flow assay has been adopted as initial screening of the HIV infection.

Currently, 4^{th} generation of rapid lateral flow assay tests for antigen detection are widely used for HIV screening by detecting nucleocapsid protein (p24). However, difficulties in detecting subtypes (non-B and non-M) HIV strains, and early infant diagnosis using p24 method has been reported. A potential solution to overcome this problem could be detecting other antigens such as envelope protein (Env) mediating the cell entry to accompany with 4^{th} generation sensing method. Several studies have shown the sensitive detection of Env protein using antibody or ConA lectin in various sensing platforms including microcantilevers, microfluidics with quantum dot, magnetic bead-based fluorescence sensing, and electrochemical impedance spectroscopy (EIS). However, the great challenge in detecting envelope protein lies in the rapid mutation of HIV. This high mutation rate comes from the molecular characteristics of HIV, namely the lack of proofreading in RNA-dependent DNA polymerases when reverse transcribing viral RNA genome. This feature gives HIV its high mutation rate, leading to individuals infected with single subtype of HIV having extensively diversified viruses in the progress of infection. Therefore, key mutations on the Env protein could result in reduced sensitivity of antibodies and escape from sensing.

Glycans, also known as polysaccharides, are an attractive biopolymer that can be utilized as a bioreceptor. Many viruses facilitate their attachment or entry into the cell by hijacking the glycans of glycocalyx. Several glycans including sialic acids, glycosaminoglycans (GAGs), and noncharged oligosaccharides are known to interact with various viruses. For example, sialic acid interacts with influenza virus and heparan sulfate, and a major component of the GAGs has been known to interact with SARS-Cov-2. Based on these findings, glycans have recently been highlighted in virus sensing applications such as influenza, Dengue, and SARS-CoV-2 viruses. HIV is one of the many viruses that hijacks the GAGs for their entry into the cell. HIV infection is initiated by the interaction between Env protein and the host cell receptor, CD4. Binding of CD4 to Env protein leads to the conformation change on Env protein to co-bind with chemokine receptors such as CCR5 or CXCR4 for host cell entry. HIV binding to heparan sulfate is thought to increase infectivity by favoring viral particle concentration at the cell surface and it can help the host cell entry by compensating the low expression of CD4 receptor on the cell surface. The interaction between heparan sulfate and Env is driven by electrostatic interactions, especially through the v3 loop region, where positive amino acids are densely populated. Moreover, heparan sulfate also can bind to the CD4i region of Env, where it is exposed only when Env is bound to CD4 through the conformational change of Env. Therefore, utilizing heparan sulfate could potentially resolve the challenge of sensing ever mutating HIV Env protein.

Here, inspired by the cellular entry mechanism of HIV, we show that glycan-virus interactions can be exploited for developing lateral flow assays to detect HIV. In particular, interaction between HS and Env protein was utilized to detect monomeric and trimeric Env proteins. We could selectively and sensitively detect the Env protein in buffer and human serum conditions along with high stability in elevated temperatures. Finally, we show the potential of sensing HIV subtypes.

### Heparan Sulfate is Optimal Capture Probe for HIV Detection

HIV envelope glycoprotein (Env) is a viral protein that engages with CD4 receptors for host cell entry. Env is initially produced as a precursor protein gp160 and it is cleaved by host cell proteases (furin) into two subunits, gp120 and gp41. Those two subunits are non-covalently associated to form metastable monomer. When three monomers (i.e., total six subunits, three gp120 and three gp41) are bound together, then it forms the trimeric envelope protein. To grasp all the binding sites on the envelope protein, we utilized the ectodomain of the Env, gp140 (monomeric, Clade CRF07_BC), to measure the binding affinity. As heparan sulfate (HS) and heparin (HEP) is composed of the same building blocks with different sulfation patterns (i.e., heparan sulfate is less sulfated and heparin is highly sulfated), we evaluated the binding affinity of HS and HEP to find the optimum capture probes for Env protein detection. Estimated binding affinity of HS and HEP were 142 nM [95% CI: 100-202 nM] and 209 nM [95% CI: 78.6-812 nM], respectively (FIG. 41, Panel B). Demonstrating that both sugars can bind to the Env protein with relatively high affinities and can be utilized as a capture probe for HIV detection. Considering that electrostatic interaction is the major driving force of GAGs, HEP can be expected to show higher binding affinity since HEP (2.3 sulfation per disaccharide) has higher negative charges on HS (0.8 sulfation per disaccharide). However, slightly lower binding affinity of HEP might come from a decrease in Kon due to the heavy glycosylation on Env impeding the association of heparin due to repulsion between negative charges on glycans on Env.

Since both sugars showed high affinity to Env protein, signal intensities were compared in lateral flow assay platform (LFSA) to find the optimal capture probe in actual testing conditions for HIV detection. Here, we have used CD4 receptors as a signaling probe, which is native host receptor for HIV entry. Utilization of CD4 as a signaling probe can provide two advantages (1) it could bind to the various subtypes; and (2) binding of CD4 to Env protein induces the structural change of Env which exposes the CD4i region where heparan sulfate can bind. Therefore, we utilized the CD4 receptor to pair with glycans. Both HS and HEP were immobilized on the nitrocellulose membrane and signal intensities were compared (FIG. 41, Panel B). As expected, HS showed significantly higher signal intensity than the HEP (p<0.05). This high signal intensity could be explained by two reasons 1) the higher binding affinity of HS to Env protein than HEP, and 2) higher immobilization of HS onto the nitrocellulose membrane than HEP due to the charge difference. HEP has a high negative charge, which can repulse with negatively charged nitrocellulose membrane. Therefore, HS was chosen as an optimal capture probe for HIV detection.

### Analytical Performance of GlycoGrip Against the HIV

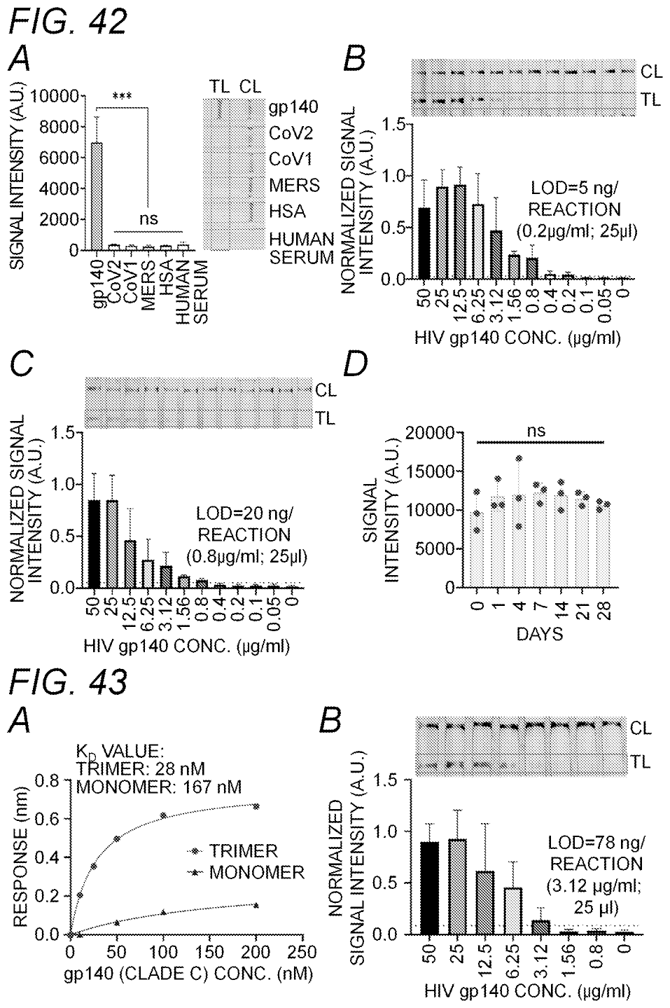

Since HS showed robust and rigorous LFSA bands, we tested its analytical performance. HS was used as a primary capture probe for the test line, and gold nanoparticle labeled CD4 was used as a reporting probe. The concentration of HS was optimized to 300 μM (data not shown). Firstly, we tested the selectivity of our biosensor against viral proteins (SARS-CoV2 spike, SARS-CoV spike and MERS-CoV spike), as well as biologically relevant proteins likely to be found in patient samples (Human serum albumin) and human serum to check the feasibility for real sample applications of the biosensor. As shown in FIG. 42, Panel A, a significant positive response was observed only in the Env (Clade CRF07_BC) protein, whereas other proteins did not show positive results. These results suggest that our sensor can selectively detect the HIV in biologically complex samples with minimized false positive signals. There was a decrease in the signal intensity in the control line when treated with human serum indicating that there might be a possible non-specific interaction between CD4 and proteins in human serum.

The analytical performance of the heparan sulfate based lateral flow strip biosensors for HIV was studied by testing different concentrations (50, 25, 12.5, 6.25, 3.12, 1.56, 0.8, 0.4, 0 μg/ml) of the Env protein (monomeric gp140; Group M; CRF07_BC) in the buffer. The Env protein was detectable as low as 39 ng/reaction (1.56 μg/ml; 25 μl) with a detectable range of 39-1250 ng/reaction (1.56-50 μg/ml) by the naked eye (data not shown). To further enhance the sensitivity of our GlycoGrip LF biosensor, we incorporated a silver enhancement process. The enhancement was carried out using silver lactate (0.3% in water) and hydroquinone (3% in 10 mM citric buffer, pH 4.0) solutions. Through the catalytic reaction, silver ions were nucleated and grew on AuNP which enhances the signal intensity on the test line. The time for the silver enhancement reaction was optimized for buffer and serum conditions to give a maximum signal (data not shown). As shown in FIG. 42, Panel B, The LOD with silver enhancement was estimated to be 5 ng/reaction (0.2 μg/ml; 25 μl), enhanced 8-fold compared with unamplified results (data not shown) which is in low nanomolar range. This shows the glycan can be utilized for sensitive detection of HIV. Further, to test feasibility of GlycoGrip in complex media, we spiked a range of Env protein concentrations into human serums (FIG. 42, Panel C). When tested with human serum spiked samples, there was a decrease in the signal intensity both of test line and control line, more severely in control line, indicating that there is a possible non-specific interaction between CD4 and matrix of proteins in human serum. However, the LOD was 78 ng/reaction (3.12 μg/ml; 25 μl), comparable to LOD of the unamplified buffer condition (data not shown). With the signal enhancement method, the Env protein was detectable as lower as 20 ng/reaction in human serum suggesting that our sensor can be suitable for clinical sample applications with high accuracy.

### GlycoGrip is Stable at Elevated Temperatures

Sensor stability is another important factor to be considered in real world sensor application. Southern Africa has 9 of the top 10 HIV percentages per population (with Equatorial Guinea at the 10th spot). In some portions of these regions, temperatures reach 40° C., and there is a projected increase in temperature between 2° C.-4.2° C. by the year 2100. Additionally, these regions face an unstable electricity supply, making refrigeration very challenging, even in healthcare facilities. Therefore, stability at the elevated temperatures should be considered. Moreover, it has been reported that elevated temperatures could impair the sensitivity of the current rapid kits for HIV detection. Therefore, stability of the sensor in elevated temperatures (40° C.) was tested. To check the stability at elevated temperatures, we put our sensor strips into a plastic bag with desiccant and stored the strips in 40° C. The signal intensity was monitored over a period of 1 month (0, 1, 4, 7, 14, 21, 28 days) and changes in signal intensity were analyzed. For 28 days, we saw no significant decrease in signal intensity. This indicates that our sensor is stable for at least 28 days in 40° C. (FIG. 42, Panel D). Suggesting that our sensor can be apt to be utilized in high HIV prevalence regions.

### Sugar as an Ideal Capture Probe for HIV Detection

So far, we have tested our sensor with monomeric form of the Env protein. However, Env protein in HIV presents as a homotrimer protein. Trimeric proteins will have a different structural profile from monomer. It has been shown that monoclonal antibodies generated from monomeric form showed a poor neutralization effect. This suggests that epitope exposure can be different between monomer and trimer, which can affect the sensing capability. Therefore, testing with trimeric Env protein is important for checking feasibility of the sensors effectiveness with virus samples. As a first step, we evaluated the binding affinity of HS to trimeric Env (Group M; Clade C) (FIG. 43, Panel A). The estimated binding affinity was 28 nM which was higher than the monomeric form of the Env protein. Increased binding affinity might be due to the multivalent interaction between the HS and the trimeric Env protein, showing the advantage of using sugar as a capture probe for virus detection. Finally, to check the analytical performance for the trimeric form of Env, we tested various concentrations of Env. As shown in FIG. 43, Panel B, The LOD was estimated to be 78 ng/reaction (3.12 μg/ml; 25 μl). This suggests that our sensor can successfully detect the trimeric form of Env.

### Heparan Sulfate can Binds to the Variants of HIV-1 Trimeric Env Protein

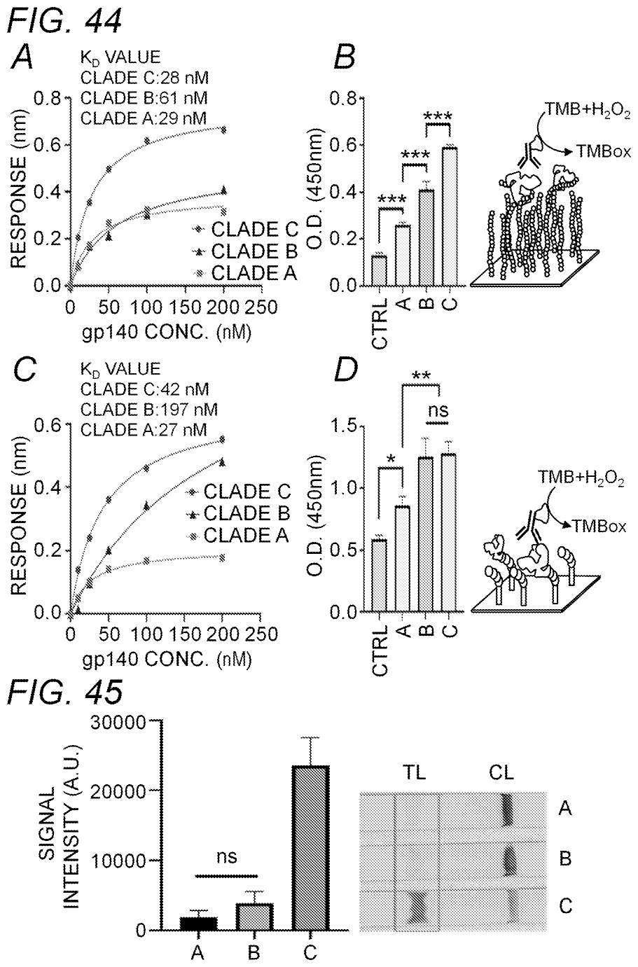

HIV has one of the highest mutation rates in known viruses. Due to this high mutation rate, HIV is classified into 4 groups (M, N, O and P), and group M can be further divided into 9 subtypes. Moreover, HIV can have a combined form of subtypes, known as the circulating recombinant form (CRF). Recently, it has been reported that highly virulent variant of HIV-1 is circulating in the Netherlands showing that HIV continues to mutate, necessitating a re-evaluation of sensing systems to keep up with mutations. Moreover, even a single subtype of the virus can generate multiple populations of the virus within a single host. Therefore, it is important to detect variants of Env proteins for robust sensing. To check whether our LFSA system can sense HIV-1 variants, we measured the binding affinities of HS to two more subtypes (clade A and B) using BLI and ELISA. Binding affinity of HS to all the subtypes were in the nanomolar range. The highest binding affinity was observed for Clade C followed by the Clade A and B (FIG. 44, Panel A). Interestingly, there was a difference in responses between subtypes, and similar trend was observed in ELISA (FIG. 44, Panel B). This suggests that there might be a difference in maximum number of bindings of Env proteins between subtypes to HS. This is likely due to the origin of Env protein and tropism, with different distribution of positive charges, which might affect HS binding.

To complete the sandwich-type binding profile, CD4 binding affinity to trimeric Env was also measured (FIG. 44, Panel C). Binding affinity of CD4 to all the subtypes were within the range of 26 nM-42 nM, except for Clade B, which was around 200 nM. As we observed in the HS case, there was also signal intensity variation between subtypes. Clade A showed weaker binding intensity than Clade C and B. Corresponding results to BLI were also obtained with ELISA, showing that CD4 binds to the all the variants, with reduced binding for clade A (FIG. 44, Panel D). This suggests that the total number of bindings of CD4 to Env protein is varied between the subtypes as was shown with HS binding.

### GlycoGrip can Potentially Detect the Presence of HIV-1 Variants

Finally, we tested all the subtypes on our LFSA system to see whether we can capture them. As shown in FIG. 45, we could observe the positive bands from all the subtypes, suggests that our sensing system can detect all the subtypes of HIV. However, there was a signal difference between variants that subtype B and A showed lower signal intensity than subtype C. This varied signal intensity on LFSA can be explained by their signal intensity difference both of HS and CD4 as we seen in BLI and ELISA (FIG. 44). Since certain amounts of gold nanoparticles should be accumulated to visualize the red color development on the test line, the total number of bound Env protein to HS and CD4 is important in signal generation. In other words, binding affinity, which determines how strong it binds together, as well as a high Bmax, which determines the maximum number of bindings, is important for signal generation. Therefore, even though binding affinities were similar between subtypes both of HS and CD4, clade C showed significantly highest signal followed by clade B and clade A. Thus, both parameters (affinity and Bmax) should be considered when utilizing the glycan for the sensing application.

In this study, we have shown that heparan sulfate can be utilized as a primary capture probe to detect HIV by pairing it with CD4 receptor, host cell receptor. By harnessing the glycan, we could sensitively and selectively detect the monomeric and trimeric form of Env including various subtypes with high stability. Moreover, we have discovered a design rule when applying glycan in sensing that both binding constant and Bmax should be taken into account. We observed that high binding affinity with lower Bmax could result in low signal intensity in lateral flow assay. Without wishing to be bound to any particular theory, for sensing using a rapid kit, the glycan should have a high binding affinity and Bmax.

### Example 5

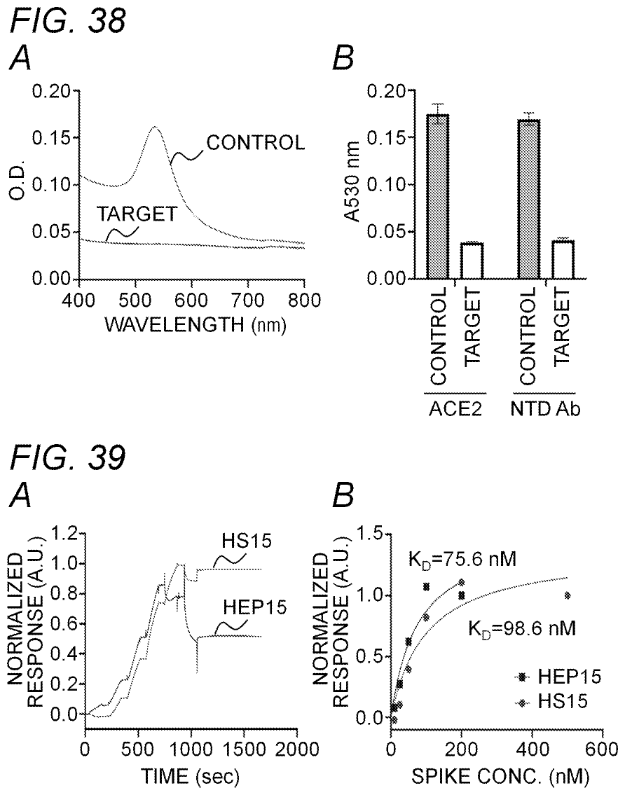

GAGs were immobilized onto a gold substrate (i.e., a plane rectangular gold coated substrate) through a biotin-avidin reaction. The gold substrate was modified with streptavidin and biotin-GAGs were introduced to the gold substrate, thereby providing GAGs that were attached to the gold substrate. Various concentrations of SARS-CoV-2 target were introduced to the GAG coated substrate. Signal change was measured optically using a surface plasmon resonance technique with the results shown in FIG. 39.

The foregoing is illustrative of the present invention and is not to be construed as limiting thereof. The invention is defined by the following claims, with equivalents of the claims to be included therein.

1. A test strip comprising:
a substrate including a test region, wherein the test region comprises a glycosaminoglycan (GAG) (e.g., a heparan sulfate proteoglycan (HSPG)) attached to the substrate, optionally wherein the substrate is configured to move a fluid sample by capillary action through the substrate to the test region.

2. The test strip of claim 1, wherein the GAG is selected from the group consisting of heparin, heparan sulfate (e.g., heparan sulfate proteoglycan (HSPG)), chondroitin sulfate, dextran sulfate, and any combination thereof, optionally wherein the GAG has a molecular weight in a range of about 1, 5, or 10 kDa to about 20, 25, 30, 40, 50, 60, 70, 80, 90, or 100 kDa, optionally wherein the GAG comprises heparin that is attached to the substrate, optionally wherein the heparin has a molecular weight in a range of about 10 kDa to about 20 kDa or wherein the GAG comprises heparan sulfate that is attached to the substrate, optionally wherein the heparan sulfate has a molecular weight in a range of about 10 kDa to about 20 kDa.

3-4. (canceled)

5. The test strip of claim 1, further comprising a sample pad that is in contact with the substrate and the sample pad is configured to be contacted by the fluid sample and is configured to move the fluid sample by capillary action through the sample pad to the substrate.

6. The test strip of claim 1, further comprising a conjugate pad that comprises a detection agent, optionally wherein the detection agent comprises an agent (e.g., a protein such as an antibody, a peptide, a GAG, and/or a nucleic acid) coupled to a signaling agent, optionally wherein the detection agent comprises an antibody labeled gold nanoparticle, optionally wherein the detection agent comprises a spike specific monoclonal antibody labeled gold nanoparticle.

7. (canceled)

8. The test strip of claim 6, wherein the detection agent is configured to bind a pathogen, optionally wherein the pathogen is a virus (e.g., a virus with a glycoprotein), optionally wherein the pathogen is a virus in the Coronaviridae family, optionally wherein the virus is a beta-coronavirus; or wherein the pathogen is severe acute respiratory coronavirus 2 (SARS-CoV-2) or a variant thereof, optionally wherein the variant is the SARS-CoV-2 Alpha strain, SARS-CoV-2 Beta strain, SARS-CoV-2 Gamma strain, SARS-CoV-2 Delta strain, SARS-CoV-2 Omicron strain, or a variant thereof (e.g., an emerging variant).

9-10. (canceled)

11. The test strip of claim 6, wherein the detection agent is configured to bind a viral spike protein, optionally wherein the detection agent is configured to bind a beta-coronavirus spike protein (e.g., a SARS-CoV spike and/or MERS-CoV spike).

12. The test strip of claim 6, wherein the detection agent comprises a protein that is selected from the group consisting of a REGN10933, a monomeric and/or dimeric ACE2, an NTD antibody, a S2M11 antibody, a 1A9 antibody, and/or a fragment thereof, optionally wherein the detection agent is the NTD antibody or monomeric and/or dimeric ACE2.

13. The test strip of claim 6, wherein the detection agent comprises a signaling agent that is selected from a gold particle (e.g., a gold nanoparticle), a latex particle (e.g., a latex microsphere), a quantum dot, a nanodiamond, a magnetic bead, an upconversion nanoparticle, a fluorescent dye, a silver nanoparticle, a nanocellulose bead, and any combination thereof.

14. The test strip of claim 8, wherein the GAG has a binding affinity for the pathogen of about 215 nM K_{D} or less (e.g., about 200, 150, 100, 75, 50, 25 nM K_{D} or less), optionally wherein the binding affinity for the GAG and pathogen is about 10 nM K_{D} to about 25 nM K_{D}.

15. The test strip of claim 8, wherein a first signal (e.g., a first color) is provided at the test region when the pathogen is bound to the GAG and the detection agent is bound to the pathogen.

16. The test strip of claim 1, wherein the substrate further comprises a control region and the control region comprises a control agent (e.g., an anti-IgG antibody) attached to the substrate and the substrate is configured to move the fluid sample by capillary action through the substrate to the control region; optionally wherein the control agent binds the detection agent and a second signal (e.g., a second color) is provided at the control region when the detection agent is bound to the control agent.

17. (canceled)

18. The test strip of claim 1, wherein the test strip has a sensitivity and/or time to detection (e.g., a signal at the test region 14 and/or control region 18) of about 30 minutes or less, responsive to contacting the fluid sample to the substrate and/or sample pad, optionally wherein the first signal is provided within about 30 minutes or less, responsive to contacting the fluid sample to the substrate and/or sample pad.

19. The test strip of claim 8, wherein the test strip has a limit of detection for the pathogen of about 80 ng/reaction or less (e.g., about 75, 60, 50, 40, 30, or 20 ng/reaction or less), optionally wherein the test strip has a limit of detection for the pathogen of about 20 ng/reaction or less.

20. The test strip of claim 1, wherein the test strip is shelf-stable for at least 30, 40, 45, or 50 days (optionally at a temperature in a range from about 20° C. to about 30° C., 35° C., 40° C., 45° C., or 50° C.), and/or wherein the test strip has a working temperature in a range from about 20° C. to about 30° C., 35° C., 40° C., 45° C., or 50° C., optionally wherein the test strip has a working temperature greater than 30° C.

21. A kit comprising:
The test strip of claim 1; and
a detection agent configured to bind a pathogen.

22-32. (canceled)

33. A method of detecting a pathogen in a fluid sample, the method comprising:
contacting the test strip of claim 1 and a fluid sample; and
detecting a first signal at the test region, wherein the first signal indicates the presence of the pathogen in the fluid sample, thereby detecting the pathogen in the fluid sample

34-51. (canceled)

52. A binding pair comprising:
a first member of the binding pair, wherein the first member comprises a glycosaminoglycan (GAG); and
a second member of the binding pair, wherein the second member comprises a detection agent that optionally includes a signaling agent,
wherein the first member and the second member are each configured to bind to the same target.

53-74. (canceled)

75. A test strip comprising the binding pair of claim 52.

76. A kit comprising the binding pair of claim 52, optionally wherein the kit comprises instructions for using the binding pair.

77-79. (canceled)

80. A method of detecting a target in a fluid sample, the method comprising:
combining a fluid sample and a binding pair of claim 52, optionally wherein the fluid sample comprises the target; and
detecting formation of a complex comprising the first member, the second member and the target, thereby detecting the target in the fluid sample.

81-89. (canceled)
